(19) United States
(12) Patent Application Publication
(10) Pub. No.: US 2025/0186405 A1
(43) Pub. Date: Jun. 12, 2025

(54) Compound For Use In Treating Narcolepsy

(71) Applicant: Alkermes, Inc., Waltham, MA (US)

(72) Inventors: Bhaskar M. REGE, Waltham, MA (US); Ishani Savant LANDRY, Bernardsville, NJ (US); Giuliano BERELLINI, Chelmsford, MA (US); Jahnavi KHARIDIA, Glen Allen, VA (US); J. Michael MacPhee, Pawtucket, RI (US); Rohit Kumar, Upton, MA (US); Jun Chen, Waltham, MA (US); Eric J. Hukkanen, Andover, MA (US); Mariya Khiterer, Melrose, MA (US); Philip C. Hogan, Allston, MA (US); Sergey A. Yagoda, Stow, MA (US)

(21) Appl. No.: 18/919,975

(22) Filed: Oct. 18, 2024

Related U.S. Application Data
(60) Provisional application No. 63/670,183, filed on Jul. 12, 2024.
(60) Provisional application No. 63/631,156, filed on Apr. 8, 2024.
(60) Provisional application No. 63/573,222, filed on Apr. 2, 2024.
(60) Provisional application No. 63/617,941, filed on Jan. 5, 2024.
(60) Provisional application No. 63/545,032, filed on Oct. 20, 2023.

(51) Int. Cl. A61K 31/4353; A61K 9/00; A61K 9/48; A61P 25/26

(52) U.S. Cl. CPC A61K 31/4353; A61K 9/0053; A61K 9/4816; A61P 25/26

(57) ABSTRACT

The present invention provides compositions and methods useful for the treatment of narcolepsy, including narcolepsy type 1 and narcolepsy type 2, cataplexy, excessive daytime sleepiness, and the like in a subject in need thereof. In embodiments, the methods comprise administering to the subject an effective amount of Compound A or a pharmaceutically acceptable salt thereof, wherein Compound A or a pharmaceutically acceptable salt thereof is administered orally once or more per day.

### RELATED APPLICATIONS

This application claims the benefit of U.S. Provisional patent application Ser. No. 63/545,032, filed on Oct. 20, 2023, U.S. Provisional patent application Ser. No. 63/617,941, filed on Jan. 5, 2024, U.S. Provisional patent application Ser. No. 63/573,222, filed on Apr. 2, 2024, U.S. Provisional patent application Ser. No. 63/631,156, filed on Apr. 8, 2024, and U.S. Provisional patent application Ser. No. 63/670,183, filed on Jul. 12, 2024. The entire contents of the above-identified applications are herein incorporated by reference.

### TECHNICAL FIELD

The present invention relates to compositions and methods useful for the treatment of narcolepsy and related symptoms with a compound having orexin-2 agonist activity.

### BACKGROUND OF THE INVENTION

Narcolepsy is a severe neurological disorder characterized by excessive daytime sleepiness (EDS) and cataplexy. Additional symptoms include hypnagogic/hypnopompic hallucinations, sleep paralysis and disturbed nighttime sleep, which altogether comprise the narcolepsy symptom pentad. Stimulants (e.g. modafinil) show certain level of effect for EDS and antidepressants (e.g. clomipramine) are used for cataplexy treatment. Sodium oxybate and pitolisant treat both EDS and cataplexy. However, these therapies do not completely address the full extent and spectrum of narcolepsy symptoms in clinical practice. Moreover, Evans et al. (PNAS, 2022, “Orexin 2 receptor-selective agonist danavorexton improves narcolepsy phenotype in a mouse model and in human patients”) describes an orexin-2 agonist administered intravenously. Further, Dauvilliers et al. (New England Journal of Medicine, 2023, 389:309-321) describes use of an oral orexin-2 agonist, dosed twice per day, in narcolepsy type 1 patients, however development of this compound was suspended following the emergence of a significant safety signal in a clinical study. Thus, an orally administered treatment option for narcolepsy is desired. Further, a once a day oral dosing regimen would provide a significant advancement in the field.

Narcolepsy type 1 is associated with the loss of orexin producing neurons. The orexin receptor is a G-protein-coupled receptor that has 2 subtypes, orexin type-1 receptor (OXIR) and orexin type-2 receptor (OX2R). Upon activation, OXIR and OX2R couple with Gq protein to increase intracellular calcium (Ca²⁺) concentration. Accordingly, narcolepsy type 1 and related symptoms thereof may be treated by the administration of an orexin agonist. Further, narcolepsy type 2, including narcolepsy without cataplexy, and related symptoms thereof, may also be treated by the administration of an orexin agonist. This application discloses Compound A, a selective OX2R agonist, compositions comprising Compound A, and the use of Compound A for the treatment of narcolepsy type 1 and/or one or more symptoms of narcolepsy type 1, narcolepsy type 2 and/or one or more symptoms of narcolepsy type 2, as well as other diseases and symptoms.

Excessive daytime sleepiness, or excessive sleepiness, is characterized by persistent sleepiness and often a general lack of energy, even during the day after apparently adequate or even prolonged nighttime sleep. Excessive sleepiness can affect the ability to function in a family, social, occupational, or other settings. Current therapies do not adequately address the full extent and spectrum of excessive sleepiness in clinical practice.

Idiopathic hypersomnia (IH) is a chronic neurological disorder that results in excessive daytime sleepiness and is frequently accompanied by long nocturnal or daytime sleep, unrefreshing sleep, difficulty in awakening, cognitive dysfunction, and autonomic symptoms. Orexin levels in patients with IH are within normal limits.

### SUMMARY OF THE INVENTION

A first embodiment is a method for treating narcolepsy in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating narcolepsy type 1 in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating narcolepsy type 2 in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating excessive daytime sleepiness and cataplexy in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating excessive daytime sleepiness and cataplexy in a subject with narcolepsy, comprising administering to the subject an effective amount of Compound A or a pharmaceutically acceptable salt thereof, wherein Compound A or a pharmaceutically acceptable salt thereof is administered orally once or more per day.

Another embodiment is a method for treating excessive daytime sleepiness in a subject with narcolepsy, comprising administering to the subject an effective amount of Compound A or a pharmaceutically acceptable salt thereof, wherein Compound A or a pharmaceutically acceptable salt thereof is administered orally once or more per day.

Another embodiment is a method for treating cataplexy in a subject suffering from narcolepsy, comprising administering to the subject an effective amount of Compound A or a pharmaceutically acceptable salt thereof, wherein Compound A or a pharmaceutically acceptable salt thereof is administered orally once or more per day.

Another embodiment is a method for treating idiopathic hypersomnia in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating excessive daytime sleepiness in a subject with idiopathic hypersomnia, comprising administering to the subject an effective amount of Compound A or a pharmaceutically acceptable salt thereof, wherein Compound A or a pharmaceutically acceptable salt thereof is administered orally once or more per day.

An embodiment of the invention is a method for decreasing or treating excessive daytime sleepiness in a subject in need thereof, comprising administering to the subject an effective amount of Compound A or a pharmaceutically acceptable salt thereof, wherein Compound A or a pharmaceutically acceptable salt thereof is administered orally once or more per day.

An embodiment of the invention is a method for treating cataplexy in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for treating shift work disorder, shift work sleep disorder or jet lag syndrome in a subject in need thereof, comprising administering to the subject an effective amount of Compound A or a pharmaceutically acceptable salt thereof, wherein

Compound A or a pharmaceutically acceptable salt thereof is administered orally once or more per day.

Another embodiment is a method for increasing wakefulness in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for increasing sleep latency in maintenance of wakefulness test (MWT) in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for improving Karolinska Sleepiness Scale (KSS) rating in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for increasing wakefulness or decreasing excessive sleepiness for about 4 hours or more in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a method for decreasing or treating excessive daytime sleepiness in a subject in need thereof, comprising administering to the subject an effective amount of Compound A or a pharmaceutically acceptable salt thereof, wherein Compound A or a pharmaceutically acceptable salt thereof is administered orally once or more per day.

Another embodiment is a pharmaceutical composition comprising (a) Compound A, or a pharmaceutically acceptable salt thereof; and (b) a pharmaceutically acceptable carrier; wherein the pharmaceutical composition comprises Compound A or pharmaceutically acceptable salt thereof having a volume based particle size with a Dv90 between about 5 and about 500 microns.

Another embodiment is a pharmaceutical composition comprising Compound A or a pharmaceutically acceptable salt thereof, wherein the volume based particle size (Dv90) of the Compound A particles is between about 5 and about 500 microns, between about 5 and about 400 microns, between about 5 and about 300 microns, between about 5 and about 200 microns, between about 5 and about 100 microns, between about 5 and about 50 microns, between about 5 and about 45 microns, between about 5 and about 40 microns, between about 5 and about 35 microns, between about 5 and about 32 microns, between about 5 and about 30 microns, between about 5 and about 28 microns, between about 5 and about 26 microns, between about 5 and about 24 microns, between about 5 and about 22 microns, between about 5 and about 20 microns, between about 5 and about 18 microns, between about 5 and about 16 microns, between about 5 and about 14 microns, between about 5 and about 12 microns, between about 5 and about 10 microns, or between about 5 and about 8 microns. In another embodiment, the volume based particle size (Dv90) of the Compound A particles is between about 8 microns and about 35 microns. In another embodiment, the volume based particle size (Dv90) of the Compound A particles is between about 8 microns and about 20 microns. In another embodiment, the volume based particle size (Dv90) of the Compound A particles is between about 10 microns and about 15 microns.

In each of the embodiments described herein, Compound A is a compound having the structure:

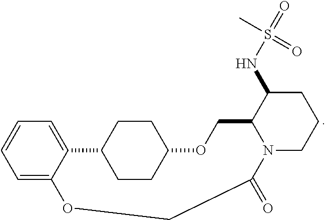

Compound A is referred to herein as N-[(4S,4aR,7α,10α)-1,2,3,4,4a,5,7,8,9,10,16,17-dodecahydro-17-oxo-7,10-ethanopyrido[1,2-d][1,7,4]benzodioxaazacyclotridecin-4-yl]methanesulfonamide.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts the overall study design for Parts 1-3 of the clinical study described in Example 2.

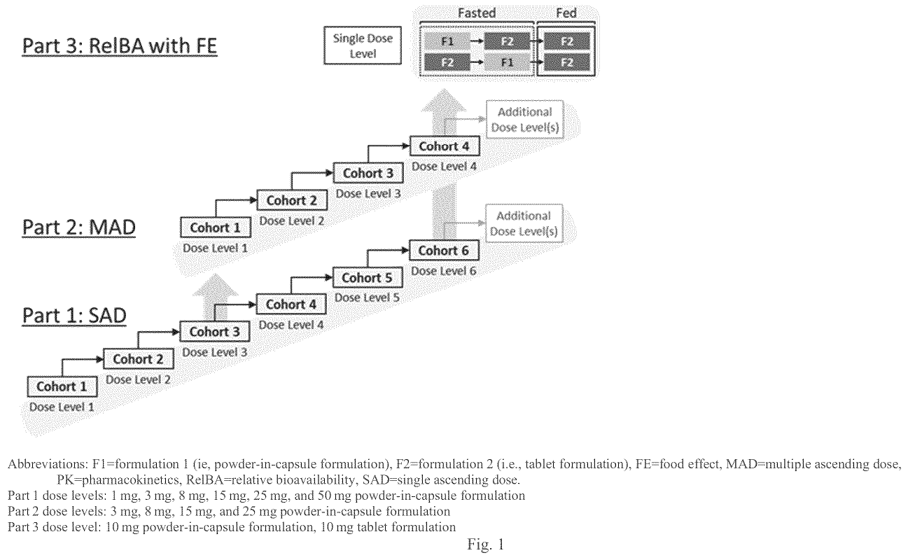

FIG. 2 depicts the overall study design for Parts 4-6 of the clinical study described in Example 2.

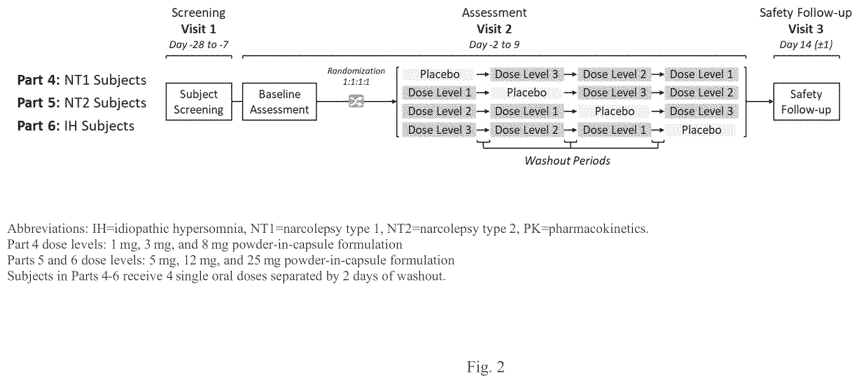

FIG. 3 depicts a plot of mean (+SD) plasma concentrations of Compound A following single oral doses of Compound A. (Abbreviations: h=hours; SAD=single ascending dose; SD=standard deviation).

FIG. 4A and FIG. 4B depict dose proportionality of PK parameters Cₘₐₓ (FIG. 4A) and AUC (FIG. 4B) following single oral doses of Compound A—Part 1 (SAD). Center line of the box represents average and the dotted line in the box represents the median. Lower edge, and upper edge of the box denote 25th, and 75th percentiles.

FIG. 5 depicts a plot of mean (+SD) plasma concentrations of Compound A following multiple oral doses of Compound A—Part 2 (MAD). (Abbreviations: h=hours; MAD=multiple ascending dose; SD=standard deviation).

FIG. 6 depicts the dose proportionality of PK parameter (AUC_{τ}) following multiple oral doses of Compound A—Part 2 (MAD).

FIG. 7 depicts a plot of mean (+SD) plasma concentrations of Compound A following tablet and capsule formulation under fasting condition, 10 mg Dose-Part 3 (RelBA with FE).

FIG. 8 depicts a plot of mean (+SD) plasma concentrations of Compound A following tablet formulation under fed (high-fat) and fasting conditions, 10 mg Dose—Part 3 (RelBA with FE) (Cohort 1).

FIG. 9 depicts mean (+SD) plasma concentrations of Compound A following single oral doses of Compound A—Part 4 (NT1) [N=4 patients].

FIG. 9A depicts mean (+SD) plasma concentrations of Compound A following single oral doses of Compound A—Part 4 (NT1) [N=10 patients].

FIG. 10 depicts a change from Day −1 (Baseline) of average sleep latency on the maintenance of wakefulness test (MWT) least squares mean (LSMEANS)+95% CI safety population. (Abbreviations: CI=confidence interval; LSMEANS=least squares mean) [N=4 patients].

FIG. 10A depicts a change from Day −1 (Baseline) of average sleep latency on the maintenance of wakefulness test (MWT) least squares mean (LSMEANS)+95% CI safety population. (Abbreviations: CI=confidence interval; LSMEANS=least squares mean) [N=10 patients].

FIG. 11 depicts maintenance of wakefulness test (MWT) change from baseline to dosing days by timepoint-mean±SE safety population. (Abbreviations: MWT=maintenance of wakefulness test; SE=standard error). Baseline is calculated as the average of first MWT measurements on Day −1. Baseline=2.94 minutes (range=1.0, 7.5). Maximum score on the MWT is 40 minutes [N=4 patients].

FIG. 11A depicts maintenance of wakefulness test (MWT) change from baseline to dosing days by timepoint-mean±SE PD population. (Abbreviations: MWT=maintenance of wakefulness test; SE=standard error). Baseline is calculated as the average of first 4 MWT measurements on Day −1. Baseline=6.13 minutes (6.41 for 8 mg group) (range=0.6, 15). Maximum score on the MWT is 40 minutes. No baseline correction [N=10 patients].

FIG. 12 depicts a PXRD pattern for the crystalline form of Compound A.

FIG. 13 depicts a DSC thermogram for the crystalline form of Compound A.

FIG. 14 depicts beta band power and participants' subjective alertness scores on the Karolinska Sleepiness Scale (KSS).

FIG. 15 depicts mean (+SD) plasma concentrations of Compound A following single oral doses of Compound A—Part 5 (NT2).

FIG. 16 depicts a change from Day −1 (Baseline) of average sleep latency on the maintenance of wakefulness test (MWT) least squares mean (LSMEANS)+95% CI PD population. (Abbreviations: CI=confidence interval; LSMEANS=least squares mean; PD=pharmacodynamic(s)).

FIG. 17 depicts maintenance of wakefulness test (MWT) change from baseline to dosing days by timepoint—mean±SE PD population. (Abbreviations: MWT=maintenance of wakefulness test; SE=standard error; PD=pharmacodynamic(s)).

FIG. 18 depicts mean (+SD) plasma concentrations of Compound A following single oral doses of Compound A—Part 6 (IH).

FIG. 19 depicts a change from Day −1 (Baseline) of average sleep latency on the maintenance of wakefulness test (MWT) least squares mean (LSMEANS)+95% CI PD population. (Abbreviations: CI=confidence interval; LSMEANS=least squares mean; PD=pharmacodynamic(s)).

FIG. 20 depicts maintenance of wakefulness test (MWT) change from baseline to dosing days by timepoint—mean±SE PD population. (Abbreviations: MWT=maintenance of wakefulness test; SE=standard error; PD=pharmacodynamic(s)).

FIG. 21 depicts a plot of mean plasma concentrations over nominal time for Compound A following tablet formulation under fed (low-fat) and fasting conditions, 10 mg Dose—Part 3 (RelBA with FE) (Cohort 2).

### DETAILED DESCRIPTION OF THE INVENTION

Provided herein are compounds or pharmaceutically acceptable salts thereof, and compositions that may modulate the orexin-2 receptor. In a particular embodiment, the compounds provided herein are considered orexin-2 agonists. As such, in one aspect, the compounds and compositions provided herein are useful in treatment of narcolepsy in a subject by acting as an agonist of the orexin-2 receptor.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in organic chemistry and clinical practice are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

As used to herein, the term “EC₅₀” refers to the concentration of a compound required to achieve an effect that is 50% of the maximal observed effect of a compound.

The term “agonist,” as used herein, refers to a compound that, when contacted with a target of interest (e.g., the orexin-2 receptor), causes an increase in the magnitude of a certain activity or function of the target compared to the magnitude of the activity or function observed in the absence of the agonist.

The term “treat,” “treated,” “treating,” or “treatment” includes the diminishment or alleviation of at least one symptom associated or caused by the state, disorder or disease being treated. In certain embodiments, the treatment comprises bringing into contact with the orexin-2 receptor an effective amount of a compound of the invention for conditions related to narcolepsy or cataplexy.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “administration” of a compound or a composition to a subject includes any route of introducing or delivering the agent to a subject to perform its intended function. Administration can be carried out by any suitable oral or non-oral route as described herein. Administration includes self-administration and the administration by another.

As used herein, “maximum plasma concentration” (Cₘₐₓ) refers to a pharmacokinetic parameter denoting the maximum measured plasma concentration following delivery of an active pharmaceutical ingredient.

As used herein, “time to maximum plasma concentration” (T max) refers to a pharmacokinetic parameter denoting the time to maximum measured plasma concentration following delivery of an active pharmaceutical ingredient, also referred to as the time to reach Cₘₐₓ.

As used herein, half-life (t_{1/2}) refers to a pharmacokinetic parameter denoting the half-life of the active pharmaceutical ingredient in the blood plasma.

As used herein, “AUC” refers to the area under the plasma concentration-time curve and can be calculated from a plasma concentration-time profile. AUC₀₋ₜ denotes the integral under the plasma concentration-time curve from time 0 (dosing) to time “t”. AUC_{0-inf} or AUC_{0-∞} denotes the AUC from time zero to time infinity.

The term “particle size” or “volume based particle size” as used herein refers to, for example, the D90 or Dv90, and means that at least about 90% of the Compound A particles have a diameter of less than the size specified. The aforementioned terms are used interchangeably herein. For example, a volume based particle size (Dv90) of less than 50 microns, means that 90% of the particle population has a diameter of less than 50 microns when measured by static or dynamic light scattering techniques known to those skilled in the art. Since the particles of the present invention tend to be irregular in shape, an approximation of the particle size is made on the basis of the volume based particle size, which specifies the diameter of the sphere that has the same volume as a given particle. Unless otherwise specified, all particle sizes are specified in terms of volume based measurements and are measured by laser light scattering/diffraction. Particle sizes are then determined based on Mie scattering theory. More specifically, unless otherwise specified, volume based particle size (Dv90) is determined using a Horiba LA-950 standard model laser particle size analyser. Deionized water is used as the sizing medium unless otherwise specified. The terms “D50” and “D10” mean that, respectively at least about 50% and 10% of the Compound A particles have a diameter of less than the size specified. These may also be referred to as “Dv50” and “Dv10” respectively, and these terms are used interchangeably herein.

The term “mean particle size” is essentially the same as “volume mean diameter” and in the present application this is defined in the same manner as defined in the Horiba Scientific brochure, “A guidebook to particle size analysis” (2012), available from Horiba's website, www.horiba.com. The calculation is expressed by conceptualizing a histogram table showing the upper and lower limits of n size channels along with the percent within each channel. The Di value for each channel is the geometric mean, the square root of upper×lower diameters. For the numerator take the geometric Di to the fourth power×the percent in that channel, summed over all channels. For the denominator take the geometric Di to the third power×the percent in that channel, summed over all channels. The volume mean diameter is referred to by several names including D[4,3].

$$D [ 4 , 3 ] = \frac{{\sum }_{1}^{η} {{D}^{4}}_{i} {v}_{i}}{{\sum }_{1}^{η} {{D}^{3}}_{i} {v}_{i}}$$

The skilled person will appreciate that particle size can also be determined by other suitable measurement means, such as by volume, number, etc.), and can be measured by, for example, sedimentation flow fractionation, dynamic light scattering, disk centrifugation, and other techniques known in the art. A full description of dynamic and static light scattering techniques is provided in Chapter 5 (p. 103-138) of “Nanoparticle technology for drug delivery” edited by Ram B. Gupta and Uday B. Kompella, 2006, published by Taylor & Franceis Group and Chapter 9 (p. 140-157) of “Pharmaceutics, the science of dosage form design” edited by Michael E. Aulton, 5^{th} Ed., 2018, published by Churchill Livingstone.

The methods, compositions and uses disclosed herein include treating diseases or disorders or symptoms associated with narcolepsy or idiopathic hypersomnia (IH) in a subject in need thereof, as well as treating subjects suffering from narcolepsy or IH who have not been diagnosed with any disease or disorder.

In an embodiment, the methods, compositions and uses of this disclosure may be directed to treating narcolepsy caused by reduced levels of orexin, a neuropeptide that regulates arousal, wakefulness, and appetite. Sleep disorders, such as narcolepsy, idiopathic hypersomnia and excessive sleepiness can also occur in individuals who do not have deficiency of orexin.

Some embodiments of the invention relate to N-[(4S,4aR,7α,10α)-1,2,3,4,4a,5,7,8,9,10,16,17-dodecahydro-17-oxo-7,10-ethanopyrido[1,2-d][1,7,4]benzodioxaazacyclotridecin-4-yl]methanesulfonamide (Compound A), or a pharmaceutically acceptable salt thereof, compositions and kits comprising Compound A, or a pharmaceutically acceptable salt thereof, and methods of using Compound A, or a pharmaceutically acceptable salt thereof.

Another embodiment is a method for treating narcolepsy in a subject in need thereof, comprising administering to the subject an effective amount of Compound A, or a pharmaceutically acceptable salt thereof, wherein Compound A or a pharmaceutically acceptable salt thereof is administered orally once or more per day.

Another embodiment is a method for treating narcolepsy in a subject in need thereof, comprising administering to the subject an effective amount of Compound A, or a pharmaceutically acceptable salt thereof, wherein the plasma concentration for Compound A is about 1.5 ng/mL or more for about 4 hours or more.

Another embodiment is a method for treating narcolepsy type 1 in a subject in need thereof, comprising administering to the subject an effective amount of Compound A, or a pharmaceutically acceptable salt thereof, wherein Compound A or a pharmaceutically acceptable salt thereof is administered orally once or more per day. In some embodiments, Compound A or a pharmaceutically acceptable salt thereof is administered orally once per day.

Another embodiment is a method for treating narcolepsy type 1 in a subject in need thereof, comprising administering to the subject an effective amount of Compound A, or a pharmaceutically acceptable salt thereof, wherein the plasma concentration for Compound A is about 1.5 ng/ml or more for about 4 hours or more.

Another embodiment provides Compound A, or a pharmaceutically acceptable salt thereof for use in treating narcolepsy in a subject in need thereof. In some embodiments, the plasma concentration for Compound A is about 1.0 ng/mL or more for about 4 hours or more. In some embodiments, the plasma concentration for Compound A is about 1.5 ng/mL or more for about 4 hours or more. In some embodiments, the plasma concentration for Compound A is about 2.0 ng/mL or more for about 4 hours or more.

Another embodiment provides Compound A, or a pharmaceutically acceptable salt thereof for use in treating narcolepsy type 1 in a subject in need thereof. In some embodiments, the plasma concentration for Compound A is about 1.0 ng/mL or more for about 4 hours or more. In some embodiments, the plasma concentration for Compound A is about 1.5 ng/ml or more for about 4 hours or more. In some embodiments, the plasma concentration for Compound A is about 2.0 ng/mL or more for about 4 hours or more.

Another embodiment is a composition comprising Compound A, or a pharmaceutically acceptable salt thereof for use in treating narcolepsy in a subject in need thereof.

Additional embodiments are uses of Compound A, or a pharmaceutically acceptable salt thereof for the manufacture of a medicament for treating narcolepsy in a subject in need thereof.

Another embodiment is a method for treating narcolepsy type 2 or idiopathic hypersomnia in a subject in need thereof, comprising administering to the subject an effective amount of Compound A, or a pharmaceutically acceptable salt thereof. In some embodiments, Compound A or a pharmaceutically acceptable salt thereof is administered orally once per day.

Another embodiment is a method for treating narcolepsy type 2 or idiopathic hypersomnia in a subject in need thereof, comprising administering to the subject an effective amount of Compound A, or a pharmaceutically acceptable salt thereof, wherein the plasma concentration for Compound A is about 3.5 ng/ml or more for about 4 hours or more.

Another embodiment provides Compound A, or a pharmaceutically acceptable salt thereof for use in treating narcolepsy type 2 or idiopathic hypersomnia in a subject in need thereof. In some embodiments, the plasma concentration for Compound A is about 3 ng/mL or more for about 4 hours or more. In some embodiments, the plasma concentration for Compound A is about 3.5 ng/mL or more for about 4 hours or more. In some embodiments, the plasma concentration for Compound A is about 4 ng/mL or more for about 4 hours or more. In some embodiments, the plasma concentration for Compound A is about 4.5 ng/mL or more for about 4 hours or more. In some embodiments, the plasma concentration for Compound A is about 5 ng/ml or more for about 4 hours or more.

Another embodiment provides uses of Compound A, or a pharmaceutically acceptable salt thereof for the manufacture of a medicament for treating narcolepsy in a subject in need thereof. In some embodiments, the plasma concentration for Compound A is about 1.5 ng/mL or more for about 4 hours or more.

Another embodiment provides uses of Compound A, or a pharmaceutically acceptable salt thereof for the manufacture of a medicament for treating narcolepsy type 1 in a subject in need thereof. In some embodiments, the plasma concentration for Compound A is about 1.5 ng/ml or more for about 4 hours or more.

Another embodiment provides uses of Compound A, or a pharmaceutically acceptable salt thereof for the manufacture of a medicament for treating narcolepsy type 2 or idiopathic hypersomnia in a subject in need thereof. In some embodiments, the plasma concentration for Compound A is about 3.5 ng/mL or more for about 4 hours or more.

Another embodiment provides methods for increasing wakefulness in a subject in need thereof, comprising administering to the subject an effective amount of Compound A, or a pharmaceutically acceptable salt thereof, wherein the plasma concentration for Compound A is about 1.5 ng/mL or more for about 4 hours or more.

Another embodiment provides methods for increasing sleep latency in maintenance of wakefulness test (MWT) in a subject in need thereof, comprising administering to the subject an effective amount of Compound A, or a pharmaceutically acceptable salt thereof, wherein the plasma concentration for Compound A is about 1.5 ng/mL or more for about 4 hours or more.

Another embodiment provides methods for treating narcolepsy in a subject in need thereof, comprising administering to the subject an effective amount of Compound A, or a pharmaceutically acceptable salt thereof. In some embodiments, the plasma concentration for Compound A is about 1.5 ng/ml or more for about 4 hours or more. In some embodiments, the plasma concentration for Compound A is about 1.5 ng/mL or more for about 6 hours or more. In some embodiments, Compound A is administered orally once per day.

Another embodiment provides methods for treating narcolepsy type 1 in a subject in need thereof, comprising administering to the subject an effective amount of Compound A, or a pharmaceutically acceptable salt thereof. In some embodiments, the plasma concentration for Compound A is about 1.5 ng/ml or more for about 4 hours or more. In some embodiments, the plasma concentration for Compound A is about 1.5 ng/ml or more for about 6 hours or more. In some embodiments, Compound A is administered orally once per day.

Another embodiment provides methods for treating narcolepsy type 2 or idiopathic hypersomnia in a subject in need thereof, comprising administering to the subject an effective amount of Compound A, or a pharmaceutically acceptable salt thereof. In some embodiments, the plasma concentration for Compound A is about 3.5 ng/ml or more for about 4 hours or more. In some embodiments, the plasma concentration for Compound A is about 3.5 ng/mL or more for about 6 hours or more. In some embodiments, Compound A is administered orally once per day.

In some embodiments, the plasma concentration for Compound A is about 1.0 ng/ml or more for about 4 hours or more. In some of the embodiments disclosed herein, the plasma concentration for Compound A is about 1.5 ng/ml or more for about 4 hours or more. In some embodiments, the plasma concentration for Compound A is about 2.0 ng/mL or more for about 4 hours or more. In some embodiments, the plasma concentration for Compound A is about 2.5 ng/mL or more for about 4 hours or more. In some embodiments, the plasma concentration for Compound A is about 3.0 ng/mL or more for about 4 hours or more. In some embodiments, the plasma concentration for Compound A is about 3.5 ng/ml or more for about 4 hours or more. In some embodiments, the plasma concentration for Compound A is about 4.0 ng/mL or more for about 4 hours or more. In some embodiments, the plasma concentration for Compound A is about 4.5 ng/mL or more for about 4 hours or more.

In some embodiments, the Cₘₐₓ following oral administration of Compound A is about 5 ng/mL or less. In some embodiments, the Cₘₐₓ following oral administration of Compound A is about 10 ng/mL or less. In some embodiments, the Cₘₐₓ for oral administration of Compound A is about 15 ng/ml or less. In some embodiments, the Cₘₐₓ for oral administration of Compound A is about 20 ng/mL or less. In some embodiments, the Cₘₐₓ following oral administration of Compound A is about 25 ng/ml or less. In some embodiments, the Cₘₐₓ following oral administration of Compound A is about 30 ng/ml or less.

### Methods and Uses

Narcolepsy is often associated with excessive daytime sleepiness (EDS). Excessive daytime sleepiness as used herein is also known as excessive sleepiness or excessive need for sleep. The methods and uses disclosed herein may treat diseases or disorders or symptoms associated with narcolepsy in a subject in need thereof. In some embodiments, the excessive sleepiness is caused by any one of the following: insufficient orexin levels, insufficient quality or quantity of night time sleep; misalignments of the body's circadian pacemaker with the environment (e.g., caused by requirement to remain awake at night for employment such as shift work or personal obligations such as caretaker for sick, young or old family members), such as jet lag, shift work and other circadian rhythm sleep disorders; another underlying sleep disorder, such as narcolepsy (e.g., narcolepsy type 1, narcolepsy type 2, probable narcolepsy), sleep apnea (e.g., obstructive sleep apnea, obstructive sleep apnea with use of continuous positive airway pressure), idiopathic hypersomnia, idiopathic excessive sleepiness, and restless legs syndrome; disorders, such as clinical depression or atypical depression; tumors; head trauma; anemia; kidney failure; hypothyroidism; injury to the central nervous system; drug abuse; genetic vitamin deficiency, such as biotin deficiency; and particular classes of prescription and over the counter medication.

In some embodiments, the methods and uses herein are used to treat any one of the following: shift work disorder; shift work sleep disorder; and jet lag syndrome. In some embodiments, the methods and uses herein are used to treat any one of the following: narcolepsy type 1, narcolepsy type 2, probable narcolepsy, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; and narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); excessive daytime sleepiness in Parkinson's disease, Prader-Willi Syndrome, sleep apnea syndrome (e.g., obstructive sleep apnea, obstructive sleep apnea with use of continuous positive airway pressure) and other disorders of vigilance; residual excessive daytime sleepiness in sleep apnea syndrome (e.g., obstructive sleep apnea, obstructive sleep apnea with use of continuous positive airway pressure); and the like. Narcolepsy (e.g., narcolepsy type 1, narcolepsy type 2, probable narcolepsy) may be diagnosed by diagnostic criteria generally used in the field, e.g., the third edition of the International Classification of Sleep Disorders (ICSD-3) and the fifth edition of the Diagnostic and Statistical Manual of Mental Disorders (DSM-5). In some embodiments, the excessive sleepiness is excessive daytime sleepiness or excessive sleepiness during working hours, or excessive sleepiness or reduced quantity of sleep which is caused by requirement to remain awake at night for employment (e.g., shift work) or personal obligations (e.g., caretaker for sick, young or old family members).

In some embodiments, treating narcolepsy may comprise reducing or alleviating one or more symptoms of narcolepsy. The one or more symptoms of narcolepsy may be selected from cataplexy, excessive sleepiness, drowsiness, languor, inertness, fatigue, and sluggishness. In some embodiments, the subject suffers from the diseases or disorders or symptoms associated with narcolepsy.

Orexin, or hypocretin, is a neuropeptide that regulates arousal, wakefulness, and appetite. In some embodiments, narcolepsy may be associated with orexin deficiency. In some embodiments, narcolepsy is not associated with reduced orexin level. In some embodiments, the orexin level in the subject is not compromised or partially compromised.

The methods and uses disclosed herein may increase wakefulness and/or decrease excessive sleepiness associated with narcolepsy in a subject in need thereof. In some embodiments, wakefulness and/or decrease of excessive sleepiness is determined by electroencephalogram (EEG) and/or electromyogram (EMG). In some embodiments, wakefulness and/or decrease of sleepiness is determined by using the Maintenance Wakefulness Test (MWT). The MWT may be quantified by EEG. An electroencephalogram (EEG) is a test that detects electrical activity in the brain, for example, by using small, metal discs or electrodes attached to the scalp.

In some embodiments, the method for increasing wakefulness or decreasing excessive sleepiness for at least about 1, 2, 3, 4, 5, 6, 7, 8, 9, 10, 11, or 12 hours or more. In some embodiments, the method for increasing wakefulness or decreasing excessive sleepiness for about 1, 2, 3, 4, 5, 6, 7, 8, 9, 10, 11, or 12 hours or more. In some embodiments, the method for increasing wakefulness or decreasing excessive sleepiness for about 6 hours or more. In some embodiments, the method for increasing wakefulness or decreasing excessive sleepiness for about 8 hours or more.

The methods and uses disclosed herein may increase sleep latency in maintenance of wakefulness test (MWT) in a subject in need thereof. In some embodiments, the sleep latency in MWT increased by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% or more. In some embodiments, the sleep latency in MWT increased by about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% or more. In some embodiments, the sleep latency in MWT increased by at least about 1, 2, 3, 4, 5, 6, 7, 8, 9, 10, 11, 12, 13, 14, 15, 16, 17, 18, 19, or 20 or more minutes. In some embodiments, the sleep latency in MWT increased by about 1, 2, 3, 4, 5, 6, 7, 8, 9, 10, 11, 12, 13, 14, 15, 16, 17, 18, 19, or 20 or more minutes.

The methods and uses disclosed herein may decrease excessive sleepiness or improve Karolinska Sleepiness Scale (KSS) rating in a subject in need thereof. In some embodiments, the KSS rating is improved 1, 2, 3, 4, or 5 or more ratings. In some embodiments, the subject has a KSS rating of 1, 2, 3, 4, or 5 after treatment with Compound A.

The methods and uses disclosed herein may comprise performing one or more tests to quantify a subject's sleepiness. In some embodiments, the test is selected from the multiple sleep latency test (MSLT), maintenance of wakefulness test (MWT), and the Oxford Sleep Resistance (OSLER) test. In some embodiments, the test is MWT. In some embodiments, the test is the Karolinska Sleepiness Scale (KSS), the Epworth Sleepiness Scale (ESS), the Stanford Sleepiness Scale, Ullanlinna Narcolepsy Scale (UNS), Work Limitations Questionnaire (WLQ), SF-8 (subset of SF-36 questionnaire) or a combination thereof.

### Modes of Administration

The methods and uses disclosed herein comprise administering Compound A to a subject in need thereof. In some embodiments, Compound A is administered orally. In some embodiments, Compound A is administered non-orally. In some embodiments, the non-oral administration is intravenous administration, subcutaneous administration, transdermal administration, intradermal administration or transmucosal administration. In some embodiments, the non-oral administration is intravenous administration. In some embodiments, the non-oral administration is subcutaneous administration. In some embodiments, the non-oral administration is transdermal administration. In some embodiments, Compound A is administered intravenously. Alternatively, or additionally, Compound A may be administered as an infusion. Administering Compound A as an infusion may comprise administering Compound A through a needle or catheter.

Compound A can be administered orally and non-orally such as intramuscular, intraperitoneal, intravenous, intraarterial, intraventricular, intracisternal injection or infusion; subcutaneous injection; or implant; or inhalation spray, intratracheal, nasal, vaginal, rectal, subdermal, transdermal, intradermal, epidural, ocular insert or ocular instillation administration, in a suitable unit dosage form containing a pharmaceutically acceptable conventional nontoxic carrier, adjuvant and vehicle suitable for each administration route.

Generally speaking, oral administration of a pharmaceutical composition may be advantageous in terms of patient convenience. Compositions of Compound A have been found to exhibit sufficient oral bioavailability to facilitate oral administration. In some embodiments, Compound A is administered as an oral dosage form. Compound A may be administered orally 1-3 times per day. Compound A may be administered orally once per day. Compound A may be administered orally twice per day. Compound A may be administered orally three times per day.

Alternatively, or additionally, administering Compound A may comprise administering an effective amount of Compound A. In some embodiments, administering Compound A may comprise administering a therapeutically effective amount of Compound A. The effective amount of Compound A may be between about 0.5 mg and about 30 mg. The effective amount of Compound A may be between about 2 mg and about 25 mg. The effective amount of Compound A may be between about 2 mg and about 20 mg of Compound A. The effective amount of Compound A may be between about 5 mg and about 25 mg. The effective amount of Compound A may be between about 2 mg and about 10 mg of Compound A. The effective amount of Compound A may be between about 5 mg and about 20 mg of Compound A. The effective amount of Compound A may be between about 8 mg and about 25 mg of Compound A. The effective amount of Compound A may be between about 2 mg and about 8 mg of Compound A. The effective amount of Compound A may be between about 2 mg and about 6 mg of Compound A.

The effective amount of Compound A may be at least 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, or at least 30 mg. The effective amount of Compound A may be at least 1 mg. The effective amount of Compound A may be at least 2 mg. The effective amount of Compound A may be at least 3 mg. The effective amount of Compound A may be at least 4 mg. The effective amount of Compound A may be at least 5 mg. The effective amount of Compound A may be at least 6 mg. The effective amount of Compound A may be at least 7 mg. The effective amount of Compound A may be at least 8 mg. The effective amount of Compound A may be at least 9 mg. The effective amount of Compound A may be at least 10 mg. The amount of Compound A may be at least 15 mg. The effective amount of Compound A may be at least 20 mg. The effective amount of Compound A may be at least 25 mg. The effective amount of Compound A may be at least 30 mg. The effective amount of Compound A may be at least 40 mg. The effective amount of Compound A may be at least 45 mg.

The effective amount of Compound A may be between about 0.5 and about 50 mg. The effective amount of Compound A may be between about 1 and about 40 mg. The effective amount of Compound A may be between about 1 and about 30 mg. The effective amount of Compound A may be between about 1 and about 25 mg. The effective amount of Compound A may be between about 1 and about 20 mg. The effective amount of Compound A may be between about 1 and about 15 mg. The effective amount of Compound A may be between about 1 and about 10 mg. The effective amount of Compound A may be between about 2 and about 10 mg. The effective amount of Compound A may be between about 2 and about 8 mg.

Depending on the subject and the period for administration of Compound A, the effective amount may change. In some embodiments, the effective amount of Compound A may be increased during the administration period of Compound A for the purpose of performing the intended or desired effect, or achieving the same or desired plasma concentration for Compound A. In some embodiments, the effective amount of Compound A is increased within the range from about 1 mg and about 8 mg of Compound A. In some embodiments, the effective amount of Compound A is increased within the range from about 5 mg and about 30 mg of Compound A.

In some embodiments, the effective amount of Compound A may be decreased during the administration period of Compound A for the purpose of performing the intended or desired effect, or achieving the same or desired plasma concentration for Compound A. In some embodiments, the effective amount of Compound A is decreased within the range from about 8 mg and about 1 mg of Compound A. In some embodiments, the effective amount of Compound A is decreased within the range from about 30 mg and about 5 mg of Compound A.

Compositions comprising about 1 mg to about 50 mg of Compound A exhibit significant plasma concentrations of the drug following oral administration whilst being generally well tolerated, see for example Tables 2-4 below.

### Pharmacokinetic Parameters

For some diseases and conditions, such as narcolepsy type 1 and related symptoms, the effective plasma concentration for Compound A is about 1-2 ng/mL for about 4 hours or more. In some embodiments, the effective plasma concentration for Compound A for the treatment of narcolepsy type 1 is about 1-2 ng/mL or more for about 4 hours or more. In some embodiments, the effective plasma concentration for Compound A for the treatment of narcolepsy type 1 is about 1-2 ng/mL or more for about 6 hours or more. In some embodiments, the effective plasma concentration for Compound A for the treatment of narcolepsy type 1 is about 1-2 ng/ml or more for about 8 hours or more. In some embodiments, the effective plasma concentration for Compound A for the treatment of narcolepsy type 1 is about 1-2 ng/mL or more for about 10 hours or more. In some embodiments, the effective plasma concentration for Compound A for the treatment of narcolepsy type 1 is about 1-2 ng/ml or more for about 12 hours or more.

For other diseases and conditions, such as narcolepsy type 2 or idiopathic hypersomnia and related symptoms, the effective plasma concentration for Compound A is about 3-4 ng/ml for about 4 hours or more. In some embodiments, the effective plasma concentration for Compound A for the treatment of narcolepsy type 2 or idiopathic hypersomnia is about 3-4 ng/ml or more for about 4 hours or more. In some embodiments, the effective plasma concentration for Compound A for the treatment of narcolepsy type 2 or idiopathic hypersomnia is about 3-4 ng/mL or more for about 6 hours or more. In some embodiments, the effective plasma concentration for Compound A for the treatment of narcolepsy type 2 or idiopathic hypersomnia is about 3-4 ng/ml or more for about 8 hours or more. In some embodiments, the effective plasma concentration for Compound A for the treatment of narcolepsy type 2 or idiopathic hypersomnia is about 3-4 ng/mL or more for about 10 hours or more. In some embodiments, the effective plasma concentration for Compound A for the treatment of narcolepsy type 2 or idiopathic hypersomnia is about 3-4 ng/ml or more for about 12 hours or more.

In some embodiments, the plasma concentration profile for Compound A represents an average plasma concentration profile for a group of treated subjects and the time period of 1 hour or more begins at the time point following oral administration. In some embodiments, the plasma concentration profile for Compound A represents the plasma concentration profile for the individually treated subject and the time period of 1 hour or more begins at the time point following oral administration to that subject.

The Cₘₐₓ following oral administration of Compound A or a pharmaceutically acceptable salt thereof is about 4 ng/mL or more of Compound A for doses described herein. The Cₘₐₓ for administration of Compound A is at least about 5, 7.5, 10, 15, 20, 25, 30, 35, 40, 45, or 50 ng/mL. The Cₘₐₓ for administration of Compound A is about 50 ng/mL or less. The Cₘₐₓ for administration of Compound A is at most 50, 40, 35, 30, 25, 20, 15, 10, 9, 8, 7, 6, or 5 ng/mL.

In some embodiments, the plasma concentration for Compound A is maintained at about 3.5 ng/mL or more for about 8 to about 12 hours following oral administration. In some embodiments, the plasma concentration for Compound A is maintained at about 1.5 ng/ml or more for about 8 to about 12 hours following oral administration.

The AUC_{0-∞} following oral administration of Compound A or a pharmaceutically acceptable salt thereof is about 12 ng*hr/mL or more of Compound A for doses described herein. The AUC_{0-∞} for administration of Compound A is at least about 12, 20, 30, 40, 50, 60, 70, 80, 90, 100, 120, 140, 160, 180, 200, 220, 240, 260, 280, 300, 350, 400, 450, or 500 ng*hr/mL. The AUC_{0-∞} for administration of Compound A is about 500 ng*hr/mL or less. The AUC_{0-∞} for administration of Compound A is at most 500, 450, 400, 350, 300, 250, 200, 190, 180, 170, 160, 150, 140, 130, 120, 110, 100, 90, 80, 70, 60, 50, 40, 30, 20, 15, 12 or 10 ng*hr/mL.

In some embodiments, the AUC₀₋₂₄ (wherein AUC₀₋₂₄ is determined over 24 hours post-dose) following oral administration of Compound A is between about 12 ng*hr/mL and about 200 ng*hr/mL. In some embodiments, the AUC₀₋₂₄ following oral administration of Compound A is between about 12 ng*hr/mL and about 180 ng*hr/mL. In some embodiments, the AUC₀₋₂₄ following oral administration of Compound A is between about 12 ng*hr/mL and about 160 ng*hr/mL. In some embodiments, the AUC₀₋₂₄ following oral administration of Compound A is between about 12 ng*hr/mL and about 140 ng*hr/mL. In some embodiments, the AUC₀₋₂₄ following oral administration of Compound A is between about 12 ng*hr/mL and about 120 ng*hr/mL. In some embodiments, the AUC₀₋₂₄ following oral administration of Compound A is between about 12 ng*hr/mL and about 100 ng*hr/mL. In some embodiments, the AUC₀₋₂₄ following oral administration of Compound A is between about 12 ng*hr/mL and about 80 ng*hr/mL. In some embodiments, the AUC₀₋₂₄ following oral administration of Compound A is between about 12 ng*hr/mL and about 70 ng*hr/mL. In some embodiments, the AUC₀₋₂₄ following oral administration of Compound A is between about 12 ng*hr/mL and about 60 ng*hr/mL. In some embodiments, the AUC₀₋₂₄ following oral administration of Compound A is between about 12 ng*hr/mL and about 50 ng*hr/mL. In some embodiments, the AUC₀₋₂₄ following oral administration of Compound A is between about 12 ng*hr/mL and about 40 ng*hr/mL.

In some embodiments, the AUC_{0-∞} increases proportionally with increasing dose. In some embodiments, the Cₘₐₓ increases at a rate less than proportional with increasing dose. In some embodiments, the AUC_{0-∞} increases proportionally with increasing dose while the Cₘₐₓ increases at a rate less than proportional with increasing dose. In some embodiments, the AUC_{0-∞} increases proportionally with increasing dose upon oral administration. In some embodiments, the AUC_{0-∞} increases with increasing dose by a ratio of about 1:1 (AUC_{0-∞}:dose). In some embodiments, the AUC_{0-∞} increases with increasing dose by a ratio of about 0.9:1. In some embodiments, the AUC_{0-∞} increases with increasing dose by a ratio of about 0.8:1. In some embodiments, the AUC_{0-∞} increases with increasing dose by a ratio of about 0.7:1. In some embodiments, the AUC_{0-∞} increases with increasing dose by a ratio of about 0.6:1. In some embodiments, the Cₘₐₓ increases at a rate less than proportional with increasing dose upon oral administration. In some embodiments, the Cₘₐₓ increases with increasing dose by a ratio of about 0.4:1 (Cₘₐₓ: dose), In some embodiments, the Cₘₐₓ increases with increasing dose by a ratio of about 0.3:1. In some embodiments, the Cₘₐₓ increases with increasing dose by a ratio of about 0.2:1. In some embodiments, the Cₘₐₓ increases with increasing dose by a ratio of about 0.1:1. In some embodiments, the AUC_{0-∞} increases proportionally with increasing dose while the Cₘₐₓ increases at a rate less than proportional with increasing dose upon oral administration.

In some embodiments, Compound A exhibits dual absorption in the plasma concentration profile over 24 hours following oral administration of Compound A. In some embodiments, Compound A exhibits dual absorption in the plasma concentration profile over 24 hours following oral administration of Compound A at a dose of 2 mg or above (As measured by the amount of free form). In some embodiments, Compound A exhibits dual absorption in the plasma concentration profile over 24 hours following oral administration of Compound A at a dose at 4 mg or above. In some embodiments, Compound A exhibits dual absorption in the plasma concentration profile over 24 hours following oral administration of Compound A at a dose between 3 mg and 30 mg. In some embodiments, Compound A exhibits dual absorption in the plasma concentration profile over 24 hours following oral administration of Compound A at a dose between 3 mg and 25 mg. In some embodiments, Compound A exhibits dual absorption in the plasma concentration profile over 24 hours following oral administration of Compound A at a dose between 3 mg and 15 mg. In some embodiments, Compound A exhibits dual absorption in the plasma concentration profile over 24 hours following oral administration of Compound A at a dose between 2 mg and 10 mg. In some embodiments, Compound A exhibits dual absorption in the plasma concentration profile over 24 hours following oral administration of Compound A at a dose between 4 mg and 8 mg.

In some embodiments, the plasma concentration for Compound A following oral administration is about 3.0 ng/mL or more at 2 hours after administration and about 1.0 ng/ml or less at 12 hours after administration. In some embodiments, the plasma concentration for Compound A following oral administration is about 3.0 ng/mL or more at 2 hours after administration and about 1.5 ng/mL or less at 12 hours after administration. In some embodiments, the plasma concentration for Compound A following oral administration is about 3.0 ng/ml or more at 2 hours after administration and about 2.0 ng/mL or less at 12 hours after administration. In some embodiments, the plasma concentration for Compound A following oral administration is about 4.0 ng/mL or more at 2 hours after administration and about 1.0 ng/ml or less at 12 hours after administration. In some embodiments, the plasma concentration for Compound A following oral administration is about 4.0 ng/mL or more at 2 hours after administration and about 1.5 ng/mL or less at 12 hours after administration. In some embodiments, the plasma concentration for Compound A following oral administration is about 4.0 ng/mL or more at 2 hours after administration and about 2.0 ng/mL or less at 12 hours after administration. In some embodiments, the plasma concentration for Compound A following oral administration is about 5.0 ng/mL or more at 2 hours after administration and about 1.0 ng/ml or less at 12 hours after administration. In some embodiments, the plasma concentration for Compound A following oral administration is about 5.0 ng/mL or more at 2 hours after administration and about 1.5 ng/mL or less at 12 hours after administration. In some embodiments, the plasma concentration for Compound A following oral administration is about 5.0 ng/mL or more at 2 hours after administration and about 2.0 ng/mL or less at 12 hours after administration. In some embodiments, the plasma concentration for Compound A following oral administration is about 7.0 ng/mL or more at 2 hours after administration and about 1.0 ng/ml or less at 12 hours after administration. In some embodiments, the plasma concentration for Compound A following oral administration is about 7.0 ng/mL or more at 2 hours after administration and about 1.5 ng/mL or less at 12 hours after administration. In some embodiments, the plasma concentration for Compound A following oral administration is about 7.0 ng/mL or more at 2 hours after administration and about 2.0 ng/ml or less at 12 hours after administration. In some embodiments, the plasma concentration for Compound A following oral administration is about 7.0 ng/mL or more at 2 hours after administration and about 3.0 ng/mL or less at 12 hours after administration. In some embodiments, the plasma concentration for Compound A following oral administration is about 7.0 ng/mL or more at 2 hours after administration and about 3.5 ng/ml or less at 12 hours after administration. In some embodiments, the plasma concentration for Compound A following oral administration is about 7.0 ng/mL or more at 2 hours after administration and about 4.0 ng/mL or less at 12 hours after administration. In some embodiments, the plasma concentration for Compound A following oral administration is about 8.0 ng/mL or more at 2 hours after administration and about 1.0 ng/ml or less at 12 hours after administration. In some embodiments, the plasma concentration for Compound A following oral administration is about 8.0 ng/mL or more at 2 hours after administration and about 2.0 ng/mL or less at 12 hours after administration. In some embodiments, the plasma concentration for Compound A following oral administration is about 8.0 ng/mL or more at 2 hours after administration and about 3.0 ng/mL or less at 12 hours after administration. In some embodiments, the plasma concentration for Compound A following oral administration is about 10.0 ng/ml or more at 2 hours after administration and about 1.0 ng/ml or less at 12 hours after administration. In some embodiments, the plasma concentration for Compound A following oral administration is about 10.0 ng/ml or more at 2 hours after administration and about 2.0 ng/mL or less at 12 hours after administration. In some embodiments, the plasma concentration for Compound A following oral administration is about 10.0 ng/ml or more at 2 hours after administration and about 3.0 ng/ml or less at 12 hours after administration. In some embodiments, the plasma concentration for Compound A following oral administration is about 10.0 ng/ml or more at 2 hours after administration and about 4.0 ng/ml or less at 12 hours after administration.

In some embodiments, the plasma concentration for Compound A following oral administration is about 15.0 ng/mL or more at 2 hours after administration and about 4.0 ng/ml or less at 12 hours after administration. In some embodiments, the plasma concentration for Compound A following oral administration is about 15.0 ng/mL or more at 2 hours after administration and about 6.0 ng/mL or less at 12 hours after administration. In some embodiments, the plasma concentration for Compound A following oral administration is about 15.0 ng/mL or more at 2 hours after administration and about 8.0 ng/mL or less at 12 hours after administration. In some embodiments, the plasma concentration for Compound A following oral administration is about 20.0 ng/mL or more at 2 hours after administration and about 4.0 ng/ml or less at 12 hours after administration. In some embodiments, the plasma concentration for Compound A following oral administration is about 20.0 ng/mL or more at 2 hours after administration and about 6.0 ng/mL or less at 12 hours after administration. In some embodiments, the plasma concentration for Compound A following oral administration is about 20.0 ng/ml or more at 2 hours after administration and about 8.0 ng/ml or less at 12 hours after administration.

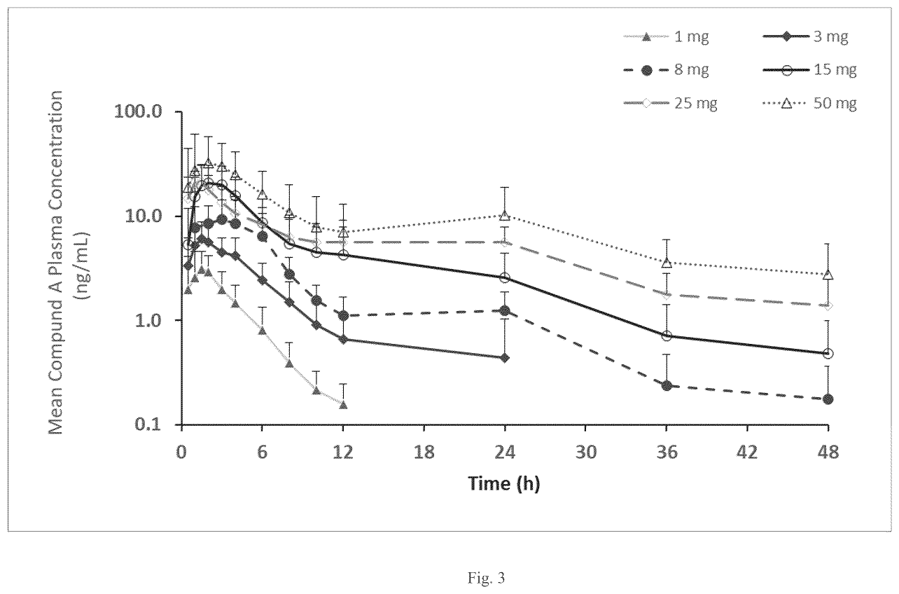

Following oral administration to subjects compositions comprising increasing doses from about 1 mg to about 50 mg of Compound A exhibit increasing Cₘₐₓ and AUC, see for example Table 4 below. At some doses a dual absorption plasma profile may be observed, see for example FIG. 3. A dual absorption profile is characterized by an initial peak in plasma concentration followed by a decline which ends in a trough which is then followed by a further increase in concentration and a second peak and subsequent decline. In some instances, the second “peak” appears as a shoulder or reduction in the rate of decrease in the declining curve (see for example the 15 mg curve in FIG. 3). Without being limited to any particular theory, such a plasma profile suggests that absorption of Compound A occurs in at least two phases—a first phase based on initial absorption post administration with the plasma concentration increasing until a point where the rate of elimination begins to dominate, leading to a first peak in concentration. As elimination continues to dominate, the plasma concentration falls. At some point a second phase of absorption inputs additional Compound A into the blood stream, reducing the rate of decrease of the plasma concentration. If the input of drug from this second phase becomes greater than the rate of elimination of drug absorbed in the first phase, then the plasma concentration may hit an inflection point and thereafter begin to rise again. When elimination of drug absorbed during the second phase (together with any drug remaining from the first phase) dominates the plasma concentration decreases for a second time. Again, without wishing to be limited by any particular theory, this type of absorption behavior may be observed where a drug substance exhibits regio-specific absorption, such that the drug is absorbed via two or more absorption windows located along the gastrointestinal tract. Alternatively, dual absorption or pulsatile plasma concentrations may be produced by design, using a dosage form adapted to release quantities of the drug at different times or rates. One approach to such a dosage form comprising populations of drug particles with different coating characteristics is described in U.S. Pat. No. 6,228,398, incorporated herein by reference, and is employed to facilitate the pulsatile plasma concentration exhibited by Ritalin LA® (methylphenidate extended-release capsules for oral administration).

### Frequency of Administration

Compound A may be administered at least 1, 2, 3 or more times per day. In some embodiments, Compound A is administered at least once per day. In some embodiments, Compound A is administered at least twice per day. In some embodiments, Compound A is administered once per day. In some embodiments, Compound A is administered twice per day. In some embodiments, Compound A is administered three times per day.

### Pharmaceutical Compositions

Further disclosed herein are pharmaceutical compositions comprising Compound A. In some embodiments, the pharmaceutical composition comprises (a) Compound A, or a pharmaceutically acceptable salt thereof; and (b) a pharmaceutically acceptable carrier therefor.

In some embodiments, the pharmaceutical composition provides a plasma concentration for Compound A of about 1.5 ng/mL or more. In some embodiments, the pharmaceutical composition provides a plasma concentration for Compound A of about 3 ng/ml or more. In some embodiments, the pharmaceutical composition provides a plasma concentration for Compound A of about 3.5 ng/mL or more. In some embodiments, the pharmaceutical composition provides a Cₘₐₓ for Compound A of about 5 ng/ml or more. In some embodiments, the pharmaceutical composition provides an effective amount of Compound A. Examples of the effective amount include between about 0.5 mg and about 50 mg, between about 2 mg and about 30 mg, and between about 2 mg and about 25 mg.

As pharmaceutically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Examples of the dosage form of the aforementioned pharmaceutical compositions include tablet (including sugar-coated tablet, film-coated tablet, orally disintegrating tablet), capsule (including soft capsule, microcapsule), granule, powder, troche, syrup, emulsion, suspension, films (e.g., orally disintegrable films), injection (e.g., subcutaneous injection, intravenous injection, intramuscular injection, intraperitoneal injection, drip infusion), external preparation (e.g., dermal preparation, ointment), suppository (e.g., rectal suppository, vaginal suppository), pellet, nasal preparation, pulmonary preparation (inhalant), eye drop and the like, which can be respectively safely administered orally or non-orally (e.g., topical, rectal, intravenous administration). These preparations may be a release control preparation (e.g., sustained-release microcapsule) such as an immediate-release preparation, a sustained-release preparation and the like.

In some embodiments, the pharmaceutical composition is formulated for oral administration. In some embodiments, the pharmaceutical composition is formulated for non-oral administration. In some embodiments, the pharmaceutical composition is formulated for intravenous administration, subcutaneous administration, transdermal administration, intradermal administration or transmucosal administration. In some embodiments, the pharmaceutical composition is formulated for intravenous administration. In some embodiments, the pharmaceutical composition is formulated for subcutaneous administration. In some embodiments, the pharmaceutical composition is formulated for transdermal administration.

### Particle Size Characterization

The particle size of the present composition may be measured using techniques such as light scattering with either water or an appropriate dilute solution as the diluent. Measurements may be verified using microscopy. Particle size distributions (PSD) may be determined using a Horiba 950 particle size analyser as a wet suspension. The volume based particle size (Dv90) is expressed herein by the mean volume diameter of the particles. Particle size measurement can also be carried out using PCS (dynamic light scattering measurements).

In addition to light scattering techniques, there are other methods for determining particle size as documented below.

Optical microscopy may be conducted on a Leica DMR microscope at 100× magnifications using Phase contrast optics. Image analysis may be performed using Axiovision software.

Scanning electron microscopy (SEM) may be conducted using a suitable scanning electron microscope such as a Phenom Pro G2. Samples may be prepared by casting diluted formulation at about 0.5 mg/mL on to 9 mm Pelcon carbon adhesive tabs, followed by air drying overnight. The samples may be sputter coated (2×) using a Denton Vacuum Desk V sputter coater.

### Further Embodiments of the Methods and Pharmaceutical Compositions Herein

In another embodiment, Compound A or a pharmaceutically acceptable salt thereof is administered orally one time to three times per day. In another embodiment, Compound A or a pharmaceutically acceptable salt thereof is administered orally once per day.

In another embodiment, Compound A or a pharmaceutically acceptable salt thereof is administered orally, in the morning. In another embodiment, Compound A or a pharmaceutically acceptable salt thereof is administered orally, once in the morning.

In another embodiment, Compound A or a pharmaceutically acceptable salt thereof is administered orally once per day at a dose of between about 0.5 mg and about 50 mg (as measured by the amount of free form). In another embodiment, Compound A or a pharmaceutically acceptable salt thereof is administered orally once per day at a dose of between about 1 mg and about 40 mg. In another embodiment, Compound A or a pharmaceutically acceptable salt thereof is administered orally once per day at a dose of between about 1 mg and about 30 mg. In another embodiment, Compound A or a pharmaceutically acceptable salt thereof is administered orally once per day at a dose of between about 1 mg and about 25 mg. In another embodiment, Compound A or a pharmaceutically acceptable salt thereof is administered orally once per day at a dose of between about 3 mg and about 25 mg. In another embodiment, Compound A or a pharmaceutically acceptable salt thereof is administered orally once per day at a dose of between about 5 mg and about 25 mg. In another embodiment, Compound A or a pharmaceutically acceptable salt thereof is administered orally once per day at a dose of between about 8 mg and about 25 mg. In another embodiment, Compound A or a pharmaceutically acceptable salt thereof is administered orally once per day at a dose of between about 8 mg and about 20 mg. In another embodiment, Compound A or a pharmaceutically acceptable salt thereof is administered orally once per day at a dose of between about 10 mg and about 25 mg. In another embodiment, Compound A or a pharmaceutically acceptable salt thereof is administered orally once per day at a dose of between about 1 mg and about 20 mg. In another embodiment, Compound A or a pharmaceutically acceptable salt thereof is administered orally once per day at a dose of between about 1 mg and about 15 mg. In another embodiment, Compound A or a pharmaceutically acceptable salt thereof is administered orally once per day at a dose of between about 1 mg and about 10 mg. In another embodiment, Compound A or a pharmaceutically acceptable salt thereof is administered orally once per day at a dose of between about 1 mg and about 8 mg. In another embodiment, Compound A or a pharmaceutically acceptable salt thereof is administered orally once per day at a dose of between about 2 mg and about 10 mg. In another embodiment, Compound A or a pharmaceutically acceptable salt thereof is administered orally once per day at a dose of between about 2 mg and about 8 mg. In another embodiment, Compound A or a pharmaceutically acceptable salt thereof is administered orally once per day at a dose of between about 1 mg and about 6 mg. In another embodiment, Compound A or a pharmaceutically acceptable salt thereof is administered orally once per day at a dose of between about 2 mg and about 6 mg.

In another embodiment, Compound A or a pharmaceutically acceptable salt thereof is administered orally once per day at a dose of about 30 mg. In another embodiment, Compound A or a pharmaceutically acceptable salt thereof is administered orally once per day at a dose of about 25 mg. In another embodiment, Compound A or a pharmaceutically acceptable salt thereof is administered orally once per day at a dose of about 20 mg. In another embodiment, Compound A or a pharmaceutically acceptable salt thereof is administered orally once per day at a dose of about 18 mg. In another embodiment, Compound A or a pharmaceutically acceptable salt thereof is administered orally once per day at a dose of about 16 mg. In another embodiment, Compound A or a pharmaceutically acceptable salt thereof is administered orally once per day at a dose of about 14 mg. In another embodiment, Compound A or a pharmaceutically acceptable salt thereof is administered orally once per day at a dose of about 12 mg. In another embodiment, Compound A or a pharmaceutically acceptable salt thereof is administered orally once per day at a dose of about 10 mg. In another embodiment, Compound A or a pharmaceutically acceptable salt thereof is administered orally once per day at a dose of about 8 mg. In another embodiment, Compound A or a pharmaceutically acceptable salt thereof is administered orally once per day at a dose of about 7 mg. In another embodiment, Compound A or a pharmaceutically acceptable salt thereof is administered orally once per day at a dose of about 6 mg. In another embodiment, Compound A or a pharmaceutically acceptable salt thereof is administered orally once per day at a dose of about 5 mg. In another embodiment, Compound A or a pharmaceutically acceptable salt thereof is administered orally once per day at a dose of about 4 mg. In another embodiment, Compound A or a pharmaceutically acceptable salt thereof is administered orally once per day at a dose of about 3 mg. In another embodiment, Compound A or a pharmaceutically acceptable salt thereof is administered orally once per day at a dose of about 2 mg. In another embodiment, Compound A or a pharmaceutically acceptable salt thereof is administered orally once per day at a dose of about 1 mg.

In another embodiment, Compound A or a pharmaceutically acceptable salt thereof is administered orally once per day, wherein the Compound A or pharmaceutically acceptable salt thereof has a particle size with a Dv90 of between about 5 and about 500 microns. In another embodiment, Compound A or a pharmaceutically acceptable salt thereof is administered orally once per day, wherein the Compound A or pharmaceutically acceptable salt thereof has a particle size with a Dv90 of between about 5 and about 400 microns. In another embodiment, Compound A or a pharmaceutically acceptable salt thereof is administered orally once per day, wherein the Compound A or pharmaceutically acceptable salt thereof has a particle size with a Dv90 of between about 5 and about 300 microns. In another embodiment, Compound A or a pharmaceutically acceptable salt thereof is administered orally once per day, wherein the Compound A or pharmaceutically acceptable salt thereof has a particle size with a Dv90 of between about 5 and about 200 microns. In another embodiment, Compound A or a pharmaceutically acceptable salt thereof is administered orally once per day, wherein the Compound A or pharmaceutically acceptable salt thereof has a particle size with a Dv90 of between about 5 and about 100 microns. In another embodiment, Compound A or a pharmaceutically acceptable salt thereof is administered orally once per day, wherein the Compound A or pharmaceutically acceptable salt thereof has a particle size with a Dv90 of between about 5 and about 50 microns. In another embodiment, Compound A or a pharmaceutically acceptable salt thereof is administered orally once per day, wherein the Compound A or pharmaceutically acceptable salt thereof has a particle size with a Dv90 of between about 5 and about 40 microns. In another embodiment, Compound A or a pharmaceutically acceptable salt thereof is administered orally once per day, wherein the Compound A or pharmaceutically acceptable salt thereof has a particle size with a Dv90 of between about 5 and about 35 microns. In another embodiment, Compound A or a pharmaceutically acceptable salt thereof is administered orally once per day, wherein the Compound A or pharmaceutically acceptable salt thereof has a particle size with a Dv90 of between about 5 and about 30 microns. In another embodiment, Compound A or a pharmaceutically acceptable salt thereof is administered orally once per day, wherein the Compound A or pharmaceutically acceptable salt thereof has a particle size with a Dv90 of between about 5 and about 25 microns. In another embodiment, Compound A or a pharmaceutically acceptable salt thereof is administered orally once per day, wherein the Compound A or pharmaceutically acceptable salt thereof has a particle size with a Dv90 of between about 5 and about 20 microns. In another embodiment, Compound A or a pharmaceutically acceptable salt thereof is administered orally once per day, wherein the Compound A or pharmaceutically acceptable salt thereof has a particle size with a Dv90 of between about 5 and about 18 microns. In another embodiment, Compound A or a pharmaceutically acceptable salt thereof is administered orally once per day, wherein the Compound A or pharmaceutically acceptable salt thereof has a particle size with a Dv90 of about 12 microns.

In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 500 microns. In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 400 microns. In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 300 microns. In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 200 microns. In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 100 microns. In another embodiment, Compound A or a pharmaceutically acceptable salt thereof is administered orally once per day at a dose of between about 0.5 mg and about 50 mg, wherein the Compound A or pharmaceutically acceptable salt thereof has a particle size with a Dv90 of between about 5 and about 50 microns. In another embodiment, Compound A or a pharmaceutically acceptable salt thereof is administered orally once per day at a dose of between about 0.5 mg and about 30 mg, wherein the Compound A or pharmaceutically acceptable salt thereof has a particle size with a Dv90 of between about 5 and about 50 microns.

In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 500 microns. In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 400 microns. In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 300 microns. In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 200 microns. In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 100 microns. In another embodiment, Compound A or a pharmaceutically acceptable salt thereof is administered orally once per day at a dose of between about 1 mg and about 25 mg, wherein the Compound A or pharmaceutically acceptable salt thereof has a particle size with a Dv90 of between about 5 and about 50 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 500 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 400 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 300 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 200 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 100 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 50 microns.

In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 500 microns. In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 400 microns. In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 300 microns. In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 200 microns. In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 100 microns. In another embodiment, Compound A or a pharmaceutically acceptable salt thereof is administered orally once per day at a dose of between about 1 mg and about 10 mg, wherein the Compound A or pharmaceutically acceptable salt thereof has a particle size with a Dv90 of between about 5 and about 50 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 500 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 400 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 300 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 200 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 100 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 50 microns.

In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 40 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 30 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of between about 5 and about 20 microns. In another embodiment, Compound A or a pharmaceutically acceptable salt thereof is administered orally once per day at a dose of between about 5 mg and about 25 mg, wherein the Compound A or pharmaceutically acceptable salt thereof has a particle size with a Dv90 of about 12 microns.

In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 40 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 30 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of between about 5 and about 20 microns. In another embodiment, Compound A or a pharmaceutically acceptable salt thereof is administered orally once per day at a dose of between about 1 mg and about 8 mg, wherein the Compound A or pharmaceutically acceptable salt thereof has a particle size with a Dv90 of about 12 microns.

In another embodiment, the volume based particle size (Dv90) of the Compound A particles is between about 5 and about 500 microns, between about 5 and about 400 microns, between about 5 and about 300 microns, between about 5 and about 200 microns, between about 5 and about 100 microns, between about 5 and about 50 microns, between about 5 and about 45 microns, between about 5 and about 40 microns, between about 5 and about 35 microns, between about 5 and about 32 microns, between about 5 and about 30 microns, between about 5 and about 28 microns, between about 5 and about 26 microns, between about 5 and about 24 microns, between about 5 and about 22 microns, between about 5 and about 20 microns, between about 5 and about 18 microns, between about 5 and about 16 microns, between about 5 and about 14 microns, between about 5 and about 12 microns, between about 5 and about 10 microns, or between about 5 and about 8 microns. In another embodiment, the volume based particle size (Dv90) of the Compound A particles is between about 8 microns and about 35 microns. In another embodiment, the volume based particle size (Dv90) of the Compound A particles is between about 8 microns and about 20 microns. In another embodiment, the volume based particle size (Dv90) of the Compound A particles is between about 10 microns and about 15 microns.

In any of the compositions described herein, where the Dv90 of the Compound A particles is between about 8 and about 500 microns, the Dv50 may be between about 3 and about 200 microns, between about 3 and about 150 microns, between about 3 and about 100 microns, between about 3 and about 75 microns, between about 3 and about 50 microns, between about 3 and about 40 microns, between about 3 and about 25 microns, between about 3 and about 20 microns, between about 3 and about 15 microns, between about 3 and about 10 microns, between about 3 and about 8 microns, between about 3 and about 6 microns, or between about 4 and about 6 microns, and the Dv10 is between about 1 and about 50 microns, between about 1 and about 40 microns, between about 1 and about 30 microns, between about 1 and about 20 microns, between about 1 and about 10 microns, between about 1 and about 8 microns, between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 35 microns, the Dv50 may be between about 3 and about 20 microns, between about 3 and about 15 microns, between about 3 and about 10 microns, between about 3 and about 8 microns, between about 3 and about 6 microns, or between about 4 and about 6 microns, and the Dv10 is between about 1 and about 10 microns, between about 1 and about 8 microns, between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 30 microns, the Dv50 may be between about 3 and about 20 microns, between about 3 and about 15 microns, between about 3 and about 10 microns, between about 3 and about 8 microns, between about 3 and about 6 microns, or between about 4 and about 6 microns, and the Dv10 is between about 1 and about 10 microns, between about 1 and about 8 microns, between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 25 microns, the Dv50 may be between about 3 and about 15 microns, between about 3 and about 10 microns, between about 3 and about 8 microns, between about 3 and about 6 microns, or between about 4 and about 6 microns, and the Dv10 is between about 1 and about 8 microns, between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 20 microns, the Dv50 may be between about 3 and about 10 microns, between about 3 and about 8 microns, between about 3 and about 6 microns, or between about 4 and about 6 microns, and the Dv10 is between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 15 microns, the Dv50 may be between about 3 and about 8 microns, between about 3 and about 6 microns, or between about 4 and about 6 microns, and the Dv10 is between about 1 and about 6 microns, between about 1 and about 5 microns, between about 1 and about 4 microns, between about 1 and about 3 microns, and between about 1 and about 2 microns.

In any of the compositions described herein, where the Dv90 of the Compound A particles is between about 8 and about 12 microns, the Dv50 may be between about 3 and about 8 microns, between about 3 and about 6 microns, or between about 4 and about 6 microns, and the Dv10 is between about 1 and about 6 microns, between about 1 and about 5 microns, between about 1 and about 4 microns, between about 1 and about 3 microns, and between about 1 and about 2 microns.

In another embodiment, Compound A or a pharmaceutically acceptable salt thereof is administered orally once per day, wherein the oral administration provides a Cₘₐₓ of Compound A of at least about 4 ng/ml within about 4 hours following oral administration. In another embodiment, Compound A or a pharmaceutically acceptable salt thereof is administered orally once per day, wherein the oral administration provides a Cₘₐₓ of Compound A of at least about 5 ng/ml within about 4 hours following oral administration. In another embodiment, Compound A or a pharmaceutically acceptable salt thereof is administered orally once per day, wherein the oral administration provides a Cₘₐₓ of Compound A of at least about 6 ng/ml within about 4 hours following oral administration. In another embodiment, Compound A or a pharmaceutically acceptable salt thereof is administered orally once per day, wherein the oral administration provides a Cₘₐₓ of Compound A of at least about 8 ng/mL within about 4 hours following oral administration. In another embodiment, Compound A or a pharmaceutically acceptable salt thereof is administered orally once per day, wherein the oral administration provides a Cₘₐₓ of Compound A of at least about 10 ng/mL within about 4 hours following oral administration. In another embodiment, Compound A or a pharmaceutically acceptable salt thereof is administered orally once per day, wherein the oral administration provides a Cₘₐₓ of Compound A of at least about 15 ng/mL within about 4 hours following oral administration. In another embodiment, Compound A or a pharmaceutically acceptable salt thereof is administered orally once per day, wherein the oral administration provides a Cₘₐₓ of Compound A of at least about 20 ng/ml within about 4 hours following oral administration.

In another embodiment, Compound A or a pharmaceutically acceptable salt thereof is administered orally once per day, wherein the oral administration provides a Cₘₐₓ of Compound A of not more than about 50 ng/mL. In another embodiment, Compound A or a pharmaceutically acceptable salt thereof is administered orally once per day, wherein the oral administration provides a Cₘₐₓ of Compound A of not more than about 40 ng/ml. In another embodiment, Compound A or a pharmaceutically acceptable salt thereof is administered orally once per day, wherein the oral administration provides a Cₘₐₓ of Compound A of not more than about 35 ng/ml. In another embodiment, Compound A or a pharmaceutically acceptable salt thereof is administered orally once per day, wherein the oral administration provides a Cₘₐₓ of Compound A of not more than about 30 ng/mL. In another embodiment, Compound A or a pharmaceutically acceptable salt thereof is administered orally once per day, wherein the oral administration provides a Cₘₐₓ of Compound A of not more than about 25 ng/mL. In another embodiment, Compound A or a pharmaceutically acceptable salt thereof is administered orally once per day, wherein the oral administration provides a Cₘₐₓ of Compound A of not more than about 20 ng/ml. In another embodiment, Compound A or a pharmaceutically acceptable salt thereof is administered orally once per day, wherein the oral administration provides a Cₘₐₓ of Compound A of not more than about 15 ng/mL. In another embodiment, Compound A or a pharmaceutically acceptable salt thereof is administered orally once per day, wherein the oral administration provides a Cₘₐₓ of Compound A of not more than about 10 ng/mL. In another embodiment, Compound A or a pharmaceutically acceptable salt thereof is administered orally once per day, wherein the oral administration provides a Cₘₐₓ of Compound A of not more than about 8 ng/ml. In another embodiment, Compound A or a pharmaceutically acceptable salt thereof is administered orally once per day, wherein the oral administration provides a Cₘₐₓ of Compound A of not more than about 7 ng/mL. In another embodiment, Compound A or a pharmaceutically acceptable salt thereof is administered orally once per day, wherein the oral administration provides a Cₘₐₓ of Compound A of not more than about 6 ng/mL. In another embodiment, Compound A or a pharmaceutically acceptable salt thereof is administered orally once per day, wherein the oral administration provides a Cₘₐₓ of Compound A of not more than about 5 ng/mL.

In another embodiment, Compound A or a pharmaceutically acceptable salt thereof is administered orally once per day, wherein the oral administration provides a Cₘₐₓ of Compound A of between about 5 ng/mL and about 35 ng/mL, and a half-life (t_{1/2}) of Compound A between about 4 and about 12 hours. In another embodiment, Compound A or a pharmaceutically acceptable salt thereof is administered orally once per day, wherein the oral administration provides a Cₘₐₓ of Compound A of between about 5 ng/ml and about 35 ng/ml, and a half-life (t_{1/2}) of Compound A between about 6 and about 12 hours. In another embodiment, Compound A or a pharmaceutically acceptable salt thereof is administered orally once per day, wherein the oral administration provides a Cₘₐₓ of Compound A of between about 5 ng/ml and about 35 ng/ml, and a half-life (t_{1/2}) of Compound A between about 8 and about 12 hours. In another embodiment, Compound A or a pharmaceutically acceptable salt thereof is administered orally once per day, wherein the oral administration provides a Cₘₐₓ of Compound A of between about 5 ng/ml and about 35 ng/ml, and a half-life (t_{1/2}) of Compound A between about 8 and about 10 hours.

In another embodiment, Compound A or a pharmaceutically acceptable salt thereof is administered orally once per day, wherein the oral administration provides a Cₘₐₓ of Compound A of between about 5 ng/ml and about 20 ng/mL, and a half-life (t_{1/2}) of Compound A between about 4 and about 12 hours. In another embodiment, Compound A or a pharmaceutically acceptable salt thereof is administered orally once per day, wherein the oral administration provides a Cₘₐₓ of Compound A of between about 5 ng/mL and about 20 ng/ml, and a half-life (t_{1/2}) of Compound A between about 6 and about 12 hours. In another embodiment, Compound A or a pharmaceutically acceptable salt thereof is administered orally once per day, wherein the oral administration provides a Cₘₐₓ of Compound A of between about 5 ng/ml and about 20 ng/ml, and a half-life (t_{1/2}) of Compound A between about 8 and about 12 hours. In another embodiment, Compound A or a pharmaceutically acceptable salt thereof is administered orally once per day, wherein the oral administration provides a Cₘₐₓ of Compound A of between about 5 ng/ml and about 20 ng/mL, and a half-life (t_{1/2}) of Compound A between about 8 and about 10 hours.

In another embodiment, Compound A or a pharmaceutically acceptable salt thereof is administered orally once per day, wherein the oral administration provides a Cₘₐₓ of Compound A of between about 20 ng/ml and about 35 ng/mL, and a half-life (t_{1/2}) of Compound A between about 4 and about 12 hours. In another embodiment, Compound A or a pharmaceutically acceptable salt thereof is administered orally once per day, wherein the oral administration provides a Cₘₐₓ of Compound A of between about 20 ng/mL and about 35 ng/ml, and a half-life (t_{1/2}) of Compound A between about 6 and about 12 hours. In another embodiment, Compound A or a pharmaceutically acceptable salt thereof is administered orally once per day, wherein the oral administration provides a Cₘₐₓ of Compound A of between about 20 ng/mL and about 35 ng/ml, and a half-life (t_{1/2}) of Compound A between about 8 and about 12 hours. In another embodiment, Compound A or a pharmaceutically acceptable salt thereof is administered orally once per day, wherein the oral administration provides a Cₘₐₓ of Compound A of between about 20 ng/ml and about 35 ng/mL, and a half-life (t_{1/2}) of Compound A between about 8 and about 10 hours.

In another embodiment, Compound A or a pharmaceutically acceptable salt thereof is administered orally once per day, wherein the oral administration provides one or more of the following:
(a) a Cₘₐₓ of Compound A of between about 4.4 ng/ml and about 27.9 ng/ml;
(b) an AUC₀₋₂₄ of Compound A of between about 19.5 ng*h/mL and about 168.5 ng*h/mL; and
(c) a Tₘₐₓ of Compound A of between about 1.5 and about 3 hours.

In another embodiment, Compound A or a pharmaceutically acceptable salt thereof is administered orally once per day, wherein the oral administration provides one or more of the following:
(a) a Cₘₐₓ of Compound A of between about 11.6 ng/ml and about 27.9 ng/ml;
(b) an AUC₀₋₂₄ of Compound A of between about 59.1 ng*h/mL and about 168.5 ng*h/mL; and
(c) a Tₘₐₓ of Compound A of between about 1.5 and about 3 hours.

In another embodiment, Compound A or a pharmaceutically acceptable salt thereof is administered orally once per day, wherein the oral administration provides one or more of the following:
(a) a Cₘₐₓ of Compound A of between about 11.6 ng/ml and about 25.0 ng/ml;
(b) an AUC₀₋₂₄ of Compound A of between about 59.1 ng*h/mL and about 150.0 ng*h/mL; and
(c) a Tₘₐₓ of Compound A of between about 1.5 and about 3 hours.

In another embodiment, Compound A or a pharmaceutically acceptable salt thereof is administered orally once per day, wherein the oral administration provides one or more of the following:
(a) a Cₘₐₓ of Compound A of between about 11.6 ng/mL and about 20.0 ng/ml;
(b) an AUC₀₋₂₄ of Compound A of between about 59.1 ng*h/mL and about 125.0 ng*h/mL; and

(c) a Tₘₐₓ of Compound A of between about 1.5 and about 3 hours.

In another embodiment, Compound A or a pharmaceutically acceptable salt thereof is administered orally once per day, wherein the oral administration provides a first plasma concentration peak of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of Compound A within about 4 hours and a second plasma concentration peak between about 10 and about 24 hours following oral administration. In another embodiment, Compound A or a pharmaceutically acceptable salt thereof is administered orally once per day, wherein the oral administration provides a first plasma concentration peak of Compound A within about 4 hours and a second plasma concentration peak between about 12 and about 24 hours following oral administration. In another embodiment, Compound A or a pharmaceutically acceptable salt thereof is administered orally once per day, wherein the oral administration provides a first plasma concentration peak of Compound A within about 4 hours and a second plasma concentration peak between about 16 and about 24 hours following oral administration. In another embodiment, Compound A or a pharmaceutically acceptable salt thereof is administered orally once per day, wherein the oral administration provides a first plasma concentration peak of Compound A within about 4 hours and a second plasma concentration peak between about 18 and about 24 hours following oral administration.

In another embodiment, Compound A or a pharmaceutically acceptable salt thereof is administered orally once per day, wherein the oral administration provides a first plasma concentration peak of between about 5 ng/mL and about 50 ng/mL of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/ml and about 45 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/ml and about 40 ng/mL of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/mL and about 35 ng/mL of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/mL and about 30 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/mL and about 25 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/mL and about 20 ng/mL of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 5 ng/mL and about 15 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 10 ng/mL and about 50 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 10 ng/mL and about 40 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 15 ng/mL and about 50 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 15 ng/mL and about 40 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 20 ng/mL and about 50 ng/ml of Compound A within about 4 hours and a second plasma concentration peak between about 8 and about 24 hours following oral administration. In another embodiment, Compound A or a pharmaceutically acceptable salt thereof is administered orally once per day, wherein the oral administration provides a first plasma concentration peak of between about 20 ng/ml and about 40 ng/mL of Compound A within about 4 hours and a second plasma concentration peak between about 8 and about 24 hours following oral administration.

### Characterization of Crystalline Form

In certain embodiments, the crystalline form described herein is identifiable on the basis of characteristic peaks in a powder X-ray diffraction analysis. Powder X-ray powder diffraction (PXRD) is a scientific technique using X-ray, neutron, or electron diffraction on powder, microcrystalline, or other solid materials for structural characterization of solid materials.

In an aspect, provided herein is a crystalline form of Compound A.

In another embodiment, the crystalline form of the Compound A is characterized by a PXRD diffractogram having peaks expressed in degrees-2-theta at angles (±0.2 degrees) of 8.95 and 10.44.

In another embodiment, the crystalline form of the Compound A is characterized by a PXRD diffractogram having peaks expressed in degrees-2-theta at angles (±0.2 degrees) of 8.95 and 11.33.

In another embodiment, the crystalline form of the Compound A is characterized by a PXRD diffractogram having peaks expressed in degrees-2-theta at angles (±0.2 degrees) of 8.95, 10.44, and 11.33.

In another embodiment, the crystalline form of the Compound A is characterized by a PXRD diffractogram having peaks expressed in degrees-2-theta at angles (±0.2 degrees) of 10.44, 12.51, and 13.81.

In another embodiment, the crystalline form of the Compound A is characterized by a PXRD diffractogram having peaks expressed in degrees-2-theta at angles (±0.2 degrees) of: 8.95, 11.33, 15.21, and 16.52.

In another embodiment, the crystalline form of the Compound A is characterized by a PXRD diffractogram having peaks expressed in degrees-2-theta at angles (±0.2 degrees) of: 10.44, 11.33, 12.51, 16.52, and 17.35.

In another embodiment, the crystalline form of the Compound A is characterized by a PXRD diffractogram having peaks expressed in degrees-2-theta at angles (±0.2 degrees) of: 8.95, 10.44, 11.33, 12.51, 13.81, and 15.21.

In another embodiment, the crystalline form of the Compound A is characterized by a PXRD diffractogram having peaks expressed in degrees-2-theta at angles (±0.2 degrees) of: 8.95, 10.44, 12.51, 13.81, 16.52, 17.92, and 20.95.

Compound A Crystalline Form

| Peak Number | Angle (degrees 2θ) |
|---|---|
| 1 | 8.95 |
| 2 | 10.44 |
| 3 | 11.33 |
| 4 | 12.51 |
| 5 | 13.81 |
| 6 | 15.21 |
| 7 | 16.52 |
| 8 | 17.35 |
| 9 | 17.92 |
| 10 | 20.95 |
| 11 | 22.54 |
| 12 | 24.94 |
| 13 | 27.93 |

In another embodiment, the crystalline form of the Compound A is characterized by any two, three, four, five, six, seven, or eight peaks from the list above.

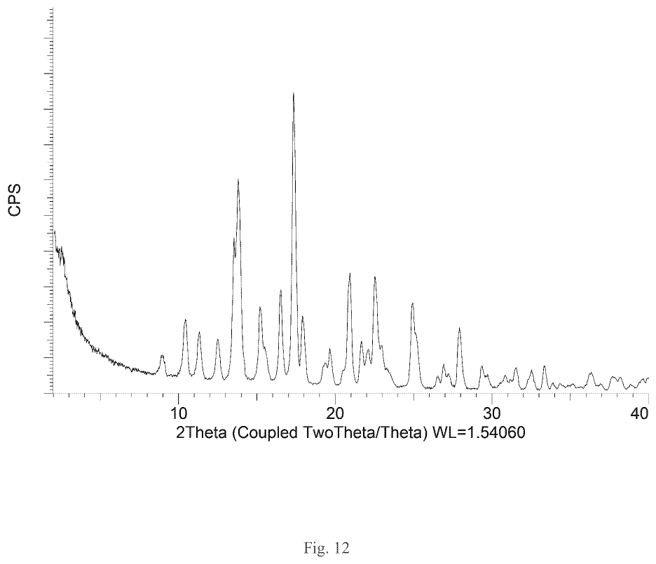

In another embodiment, the crystalline form of the Compound A has a PXRD diffractogram substantially as depicted in FIG. 12.

In another embodiment, the crystalline form of the Compound A has a DSC thermogram characterized by an endotherm with an onset temperature of 297.5 degrees C.

### Optically Active Compound

Compound A is an optically active compound. Compound A is N-[(4S,4aR,7α,10α)-1,2,3,4,4a,5,7,8,9,10,16,17-dodecahydro-17-oxo-7,10-ethanopyrido[1,2-d][1,7,4]benzodioxaazacyclotridecin-4-yl]methanesulfonamide in any of the methods, the uses and the pharmaceutical compositions disclosed herein.

The disclosed compound, i.e., Compound A, possesses one or more stereocenters, and each stereocenter may exist independently in either the R or S configuration. In one embodiment, compounds described herein are present in optically active or racemic forms. It is to be understood that the inventions described herein encompass racemic, optically-active, regioisomeric and stereoisomeric forms, or combinations thereof that possess the therapeutically useful properties described herein.

Compounds described herein are synthesized using any suitable procedures starting from compounds that are available from commercial sources or are prepared using procedures known in the art or described herein. In one embodiment, Compound A is synthesized using the procedure provided in PCT Publication No. WO2021/108628. Compound A can also be synthesized using procedures provided in PCT Publication No. WO2022/140316. Accordingly, the entire contents of WO2021/108628 and WO2022/140316 are hereby incorporated by reference in their entireties.

As further illustrated in the Examples below, when administered to healthy volunteers, compositions comprising Compound A have been shown to: (i) be well tolerated up to doses of 50 mg, (ii) increase objective and subjective measures of alertness, and (iii) exhibit PK/PD characteristics supporting once-daily oral dosing. In patients with narcolepsy type-1 compositions comprising Compound A have been shown to: (a) be well tolerated at doses of 1 mg to 8 mg, with drug-related adverse events only observed at the highest dose (8 mg), (b) exhibit statistically significant, clinically meaningful and durable improvement in sleep latency, and (c) be suitable for once-daily oral administration.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is further illustrated by the following examples, which should not be construed as further limiting. The practice of the present invention will employ, unless otherwise indicated, conventional techniques of organic synthesis or clinical use, which are within the skill of the art. Unless otherwise noted, all solutions described herein are aqueous solutions.

### List of Abbreviations

The following abbreviations are used in the synthetic examples below:
ACN=acetonitrile
DIPEA=N,N-diisopropylethylamine
g=grams
mg=milligrams
h=hours
HPLC=high-performance liquid chromatography
KF=Karl Fischer titration
MTBE=methyl tert-butyl ether
NMP=N-methylpyrrolidone
V=volume
s=seconds
min=minutes

### Summary of Methods

### Particle Size Distribution

### Instrument: Malvern Mastersizer 3000

### Sample Preparation for Measurement of Particle Size Distribution (PSD)

### For Crude Materials

˜200 mg of compound was weighed into 8 mL sample vial. 2 mL of 0.1% Tween 20 solution was added to the sample vial and then vortex for 120 s to produce a homogeneous solution. The solution was immediately added dropwise to the dispersion module using a plastic pipette. Samples were prepared in duplicate.

### For Milled Materials

˜30 mg of compound was weighed into 8 mL sample vial. 2 mL of 0.1% Tween 20 solution was added to the sample vial and then vortex for 120 s to produce a homogeneous solution. The solution was immediately added dropwise to the dispersion module using a plastic pipette. Samples were prepared in duplicate.

### Sample Measurement

500 mL of water (dispersant) was transferred to a beaker and stirring was initiated with minimum speed of 3000 rpm. Background measurement was obtained. Pre-dispersed sample solution was added to beaker containing dispersant until laser obscuration reached 5-12% and stabilized for 30 s. The sample was sonicated for 30 s and then circulated in the instrument for 90 s prior to starting measurements. Sample was measured upon ultrasound intensity reaching 50%. The instrument was then purged with water.

### Specific Surface Area

### Instrument: Micromeritics Accelerated Surface Area and Porosimetry Analyzer ASAP2020 (BET)

Procedure: In duplicates, approximately 0.5 g of material was weighed and added to a sample tube with seal frit. The sample tube was attached to the degas port. The heating jacket was firmly fastened to the sample tube. The sample was degassed using the following procedure:

| Phase | Criteria/Option | Value | |
|---|---|---|---|
| N/A | Description | Enter degas description | |
| | | (optional) | |
| Evacuation Phase | Temperature ramp rate | 1.0 | (K/min) |
| Evacuation Phase | Target temperature | 303 | (K) |
| Evacuation Phase | Evacuation rate | 0.67 | (kPa/s) |
| Evacuation Phase | Unrestricted evac. from | 0.67 | (kPa/s) |
| Evacuation Phase | Vacuum setpoint | 1.3 | (Pa) |
| Evacuation Phase | Evacuation time | 0 | (min) |
| Heating Phase | Ramp rate | 5.0 | (K/min) |
| Heating Phase | Hold temp | 323 | (K) |
| Heating Phase | Hold time | 180 | (min) |
| Evacuation and | Hold pressure | 13.3 | (kPa) |
| Heating phases | | | |

| | | |
|---|---|---|
| End of Degas | Backfill sample tube | Checked |

Prepared the analysis dewar filled with liquid Nitrogen. Post degassing step, the sample tube was removed from the degas port and was attached to the analysis port, such that the sample holder was submerged in the liquid nitrogen analysis dewar. The analysis was performed using nitrogen gas with a partial pressure range of 0.045 to 0.25 and using the ideal gas with non-ideality correction factors option. The analysis temperature was set at 77.3 K, and the saturation temperature was calculated from the analysis temperature. The nitrogen adsorption isotherm was analyzed using the BET methodology to calculate the specific surface area of a sample.

### Analytical Methods

Powder X-Ray Diffraction (PXRD) was performed on a Rigaku MiniFlex 6G diffractometer (Rigaku/MSC, Woodlands, TX) in reflection mode using collimated Cu Kα radiation operating at 40 kV/15 mA. Scans were run from 2-40 degrees 2-theta with a step size of 0.02 degrees and a scan time of 7.6 degrees 2-theta/minute.

Differential Scanning calorimetry (DSC) was conducted on a TA Instruments DSC 2500 with aluminum crimped pans. DSC analysis was performed by ramping 10 degrees C. per minute from 20 degrees to 350 degrees C.

### Example 1—Synthesis of Compound A and Characterization of Particle Size

### 1.1 Intermediate Preparation

An autoclave was charged with NMP (3.0 V) and agitated. The temperature was adjusted to 25±5 degrees C. and (2¹S,2⁴S,5²R,5³S)-5³-(benzylamino)-3,8-dioxa-5(2,1)-piperidina-1(1,2)-benzena-2(1,4)-cyclohexanacyclooctaphan-6-one (1.0 eq.) was added. The charging port was rinsed with NMP (1.0 V) and then hydrochloric acid (0.8 eq.) was added. The reaction was stirred for at least 2 hours at 25±5 degrees C. and then hydroxide palladium carbon (5% w/w) was added. The charging port was rinsed with NMP (1.0 V) and the autoclave was heated slowly while pressurizing with hydrogen until heating the autoclave to 45±5 degrees C. slowly. This procedure was repeated until the system pressure change is less than 0.1 MPa in one hour. The reaction was analyzed via HPLC every 6 hours after stirring for at least 16 hours.

The autoclave was cooled to 25±5 degrees C., charged with ACN (10 V), and then stirred for at least 1 h. The reaction was filtered and the cake was washed with ACN (2 V) and the filtrate was collected. The filtrate was transferred to a reactor via a filter and the organic phase was concentrated to 5-6 V under vacuum, controlling the internal temperature to no more than 45 degrees C. and a jacket temperature to no more than 55 degrees C. The temperature was adjusted to 25±5 degrees C., then MTBE (10 V) was added and the mixture was stirred for at least 1 h. Then 2-mercaptopropyl ethyl sulfide silica (0.2 w/w) was added and stirred for at least 16 h at 25±5 degrees C. The reaction was filtered and the cake washed with ACN (2 V). The organic phase was diluted with ACN (10 V) and concentrated to 5-6 V under vacuum, controlling the internal temperature to no more than 45 degrees C. and a jacket temperature to no more than 55 degrees C. The organic phase was then further concentrated to 4-6 V under vacuum, controlling the internal temperature to no more than 50 degrees C. and a jacket temperature to no more than 60 degrees C. to afford a solution of (2¹S,2⁴S,5²R,5³S)-5³-amino-3,8-dioxa-5(2,1)-piperidina-1(1,2)-benzena-2(1,4)-cyclohexanacyclooctaphan-6-one.

### 1.2 Compound A

A reactor under N₂ protection was charged with a solution of (2¹S,2⁴S,5²R,5³S)-5³-amino-3,8-dioxa-5(2,1)-piperidina-1(1,2)-benzena-2(1,4)-cyclohexanacyclooctaphan-6-one (1.0 eq. of (2¹S,2⁴S,5²R,5³S)-5³-(benzylamino)-3,8-dioxa-5(2,1)-piperidina-1(1,2)-benzena-2(1,4)-cyclohexanacyclooctaphan-6-one) at 20±5 degrees C. and agitated. The reactor was then charged with DIPEA (3.0 eq.) and the temperature was adjusted to 0±5 degrees C. The reactor was then charged with methanesulfonic acid (1.5 eq.) and stirred for at least 1 h at 0±5 degrees C. The reaction was monitored by HPLC. The reactor was then charged with purified water (0.3 V) at 0±5 degrees C. The reactor was then heated to 60±5 degrees C. The reactor was charged with purified water (1.0 V) over 2 h at 60±5 degrees C. The reactor was charged with seed, Compound A (0.1 wt %; PSD=Dv10=42.2 micron, Dv50=156 micron, and Dv90=265 micron), and stirred for 1 h at 60±5 degrees C. The reactor was additionally charged with purified water (1.2 V) over 2 h at 60±5 degrees C. The reactor was then cooled to 20±5 degrees C. and stirred for 2 h. The supernatant concentration was monitored by HPLC. The mixture was filtered and the cake was washed with purified water (2.0 V). The cake was dried under vacuum at 45±5 degrees C. and monitored by KF to afford Compound A as a crude solid. The particle size distribution (PSD) of the resultant material was: Dv10=29.1 micron, Dv50=185 micron, and Dv90=309 micron.

### 1.3 Milled Compound A (Dv90=87.7 Micron)

Compound A resultant material from Example 1.2 was sieved using a 30 mesh screen, then was micronized using a spiral jet mill and nitrogen supply. The jet mill used was a Pilot Mill-4, manufactured by FPS Food and Pharma Systems. The jet milling operating conditions were:
Feed rate=1.1 kg/h
Feed pressure=7 barg
Mill pressure=7 barg
   The micronization conditions yielded Compound A with a PSD of Dv10=5.66 micron, Dv50=30.9 micron, and Dv90=87.7 micron.
   1.4 Milled Compound A (Dv90=53.8 micron)

Compound A resultant material from Example 1.3 was further micronized using a spiral jet mill and nitrogen supply. The jet mill used was a Pilot Mill-4, manufactured by FPS Food and Pharma Systems. The jet milling operating conditions were:
Feed rate=0.4 kg/h
Feed pressure=7 barg
Mill pressure=7 barg
   The micronization conditions yielded Compound A with a PSD of Dv10=3.19 micron, Dv50=17.7 micron, and Dv90=53.8 micron.
   1.5 Milled Compound A (Dv90=32.4 micron)

Compound A resultant material from Example 1.4 was further micronized using a spiral jet mill and nitrogen supply. The jet mill used was a Pilot Mill-4, manufactured by FPS Food and Pharma Systems. The jet milling operating conditions were:
Feed rate=0.3 kg/h
Feed pressure=7 barg
Mill pressure=7 barg

The micronization conditions yielded Compound A with a PSD of Dv10=2.3 micron, Dv50=9.2 micron, and Dv90=32.4 micron. The specific surface area of the resultant material, measured by BET, was 5.73 m²/g.

### Example 2—Compound A (Dv90=93.0 micron)

A reactor under N₂ protection was charged with a solution of (2¹S,2⁴S,5²R,5³S)-5³-amino-3,8-dioxa-5(2,1)-piperidina-1(1,2)-benzena-2(1,4)-cyclohexanacyclooctaphan-6-one (1.0 eq. of (2¹S,2⁴S,5²R,5³S)-5³-(benzylamino)-3,8-dioxa-5(2,1)-piperidina-1(1,2)-benzena-2(1,4)-cyclohexanacyclooctaphan-6-one) through a filter and agitated. The reactor was then charged with DIPEA (3.0 eq.) through a filter and the temperature was adjusted to 0±5 degrees C. The reactor was then charged with methanesulfonic acid (1.5 eq.) and stirred for at least 2 h at 0±5 degrees C. The reaction was monitored by HPLC. The reactor was heated to 20±5 degrees C. The reactor contents were transferred to a drum.

A second reactor was charged with the reaction solution through a filter and the temperature was adjusted to 20±5 degrees C. The reactor was then charged with purified water (0.3 V) over at least 1 h at 20±5 degrees C. The reactor was then heated to 60±5 degrees C. The reactor was charged with seed, Compound A (1.0 wt %, resultant material from Example 1.5), and stirred for at least 2 h at 60±5 degrees C. The reactor was charged with purified water (2.2 V) over at least 4 h at 60±5 degrees C. The reactor was stirred for at least 1 h at 60±5 degrees C. The reactor was then cooled to 20±5 degrees C. and stirred for at least 10 h. The supernatant concentration was monitored by HPLC. The mixture was filtered and the cake was washed with purified water (5.0 V) three times. The cake was dried under vacuum at 50±5 degrees C. and monitored by KF for at least 16 h to afford Compound A as a crude solid. The PSD was determined as: Dv10=11.9 micron, Dv50=44.2 micron, and Dv90=93.0 micron.

2.1 Milled Compound A (Dv90=12 micron)

Resultant material from Example 2 was sieved using a 20-mesh screen, then was micronized using a spiral jet mill and nitrogen supply. The jet mill used was a Pilot Mill-6, manufactured by FPS Food and Pharma Systems. The jet milling operating conditions were:
Feed rate between 0.6-1.2 kg/h
Feed pressure=6 barg
Mill pressure=6 barg
   The micronization conditions yielded Compound A with a PSD of Dv10=1.4 micron, Dv50=4.7 micron, and Dv90=12 micron. The specific surface area of the resultant material, measured by BET, was 8.83 m²/g.

### Example 3—Compound A (Dv90=75.2 micron)

A reactor was charged with NMP (0.5 V) under N₂ protection and agitated. The temperature was adjusted to 25±5 degrees C. followed by the addition of methanesulfonic anhydride (1.5 eq.). The charging port was rinsed with NMP (0.5 V). The solution was stirred for at least 1 h at 25±5 degrees C. The solution in the reactor was passed through a filter and collected into a drum.

A second reactor was charged with a filtered solution of (2¹S,2⁴S,5²R,5³S)-5³-amino-3,8-dioxa-5(2,1)-piperidina-1(1,2)-benzena-2(1,4)-cyclohexanacyclooctaphan-6-one (1.0 eq. of (2¹S,2⁴S,5²R,5³S)-5³-(benzylamino)-3,8-dioxa-5(2,1)-piperidina-1(1,2)-benzena-2(1,4)-cyclohexanacyclooctaphan-6-one) under N₂ protection and agitated. The reactor was then charged with DIPEA (3.0 eq.) through a filter and the temperature was adjusted to 0±5 degrees C. Then, the solution of methanesulfonic anhydride in NMP was charged to the reactor and stirred for at least 2 h at 0±5 degrees C. The reaction was monitored by HPLC.

The reactor was charged with purified water (0.8 V) over 3 h at 0±5 degrees C. The reactor was then heated to 60±5 degrees C. The reactor was charged with seed, Compound A (1.0 wt %, resultant material from Example 1.3), and stirred for at least 2 h at 60±5 degrees C. The reactor was charged with purified water (2.2 V) over at least 4 h at 60±5 degrees C. The reactor was stirred for at least 1 h at 60±5 degrees C. The reactor was then cooled to 20±5 degrees C. and stirred for at least 10 h. The supernatant concentration was monitored by HPLC. The mixture was filtered and the cake was washed with purified water (5.0 V) three times. The cake was dried under vacuum at 50±5 degrees C. and monitored by KF for at least 16 h to afford Compound A as a crude solid. The PSD was determined as: Dv10=17.1 micron, Dv50=41.6 micron, and Dv90=75.2 micron.

3.1 Milled Compound A (Dv90=10.7 micron)

Resultant material from Example 3 was sieved using a 20-mesh screen, then was micronized using a spiral jet mill and nitrogen supply. The jet mill used was a Pilot Mill-6, manufactured by FPS Food and Pharma Systems. The jet milling operating conditions were:
Feed rate between 1.1-1.3 kg/h
Feed pressure=6 barg
Mill pressure=6 barg
   The micronization conditions yielded Compound A with a PSD of Dv10=1.43 micron, Dv50=4.53 micron, and Dv90=10.7 micron.

### Example 4—Clinical Study

A multicenter Phase 1a/b study was undertaken to assess the safety, tolerability, PK, and PD of Compound A following oral administration in healthy adult subjects and subjects with NT1, NT2, or IH.

The study drug, Compound A, was administered as either a powder-in-capsule or as a tablet. All formulations dosed were prepared using the resultant material from Example 2.1 (Dv90=12 micron).

In powder-in-capsule form, Compound A was prepared by filling size 0 Swedish orange, opaque, hydroxypropyl methylcellulose (HPMC) capsules with Compound A drug substance with no additional excipients. The capsules were filled by weighing the Compound A drug substance into the capsules prior to dosing at target fill weights ranging from 1 mg to 50 mg per capsule.

In tablet form, Compound A was formed into a round tablet containing Compound A drug substance. Dosages ranged from 1 mg to 10 mg per tablet.

The study consisted of the following parts:
Part 1 is a randomized, double-blind, placebo-controlled part to assess the safety, tolerability, and PK of Compound A following single ascending dose (SAD) administration
Part 2 is a randomized, double-blind, placebo-controlled (for Cohorts 1-4 following multiple ascending dose (MAD) administration
Part 3 is a randomized, open-label part to assess Relative Bioavailability and Food Effect of formulation(s) of Compound A following single-dose administration
Parts 4-6 are randomized, double-blind, placebo-controlled, 4-way crossover parts with 4 periods to assess the safety, tolerability, PK, and PD of Compound A following single-dose administrations in subjects with NT1 (Part 4), NT2 (Part 5), or IH (Part 6).

### 4.1 Part 1: Single Ascending Dose (SAD)

Part 1 of the study is a SAD evaluation in healthy subjects consisting of 6 cohorts: Cohorts 1-6 received powder-in-capsule or matching placebo. There were approximately 8 subjects per cohort. Dose levels were as follows:

| Part No. and | Cohort | | |
|---|---|---|---|
| Description | No. | Dose Level | Associated Study Drugs |
| Part 1 SAD | Cohort 1 | 1 mg | Powder-in-capsule and matching |
| | Cohort 2 | 3 mg | placebo |
| | Cohort 3 | 8 mg | |
| | Cohort 4 | 15 mg | |
| | Cohort 5 | 25 mg | |
| | Cohort 6 | 50 mg | |

Subjects in Part 1 were randomized 3:1 to receive Compound A: placebo treatments. Sentinel dosing of 2 subjects was implemented for each dose cohort in Part 1 to assess 24 hours of safety observations, with 1 subject treated with Compound A and 1 subject treated with matching placebo at the corresponding dose level. Any subject who was not randomized but completed Day −2 assessments and was eligible was later randomized without repeating Day −2 assessments, as long as the subject had not left the clinic.

### 4.2 Part 2: Multiple Ascending Dose (MAD)

Part 2 of the study is a MAD evaluation in healthy subjects consisting of 4 cohorts: Cohorts 1-4 received powder-in-capsule or matching placebo. Dose levels were as follows:

| Part No. and | Cohort | | |
|---|---|---|---|
| Description | No. | Dose Level | Associated Study Drugs |
| Part 2 MAD | Cohort 1 | 3 mg | Powder-in-capsule and matching |
| | Cohort 2 | 8 mg | placebo |
| | Cohort 3 | 15 mg | |
| | Cohort 4 | 25 mg | |

Subjects in Cohorts 1-4 of Part 2 were randomized 3:1 to receive Compound A: placebo treatments. Any subject who was not randomized but completed Day −2 assessments and was eligible was later randomized without repeating Day −2 assessments, as long as the subject had not left the clinic.

4.3 Part 3: Relative Bioavailability with Food Effect

Part 3 compared the relative bioavailability (RelBA) of Compound A between a tablet formulation and the powder-in-capsule formulation. In addition, the food effect (FE) on the bioavailability of Compound A was assessed following administration of tablet formulation. Dose levels were as follows:

| Part No. and | Cohort | | |
|---|---|---|---|
| Description | No. | Dose Level | Associated Study Drugs |
| Part 3 RelBA | NA | 10 mg | Powder-in-capsule and tablet |
| with FE | | | |

The Part 3 (RelBA with FE) evaluation was conducted using a 2-sequence, 3-period crossover study design in approximately 12 subjects. Subjects in Part 3 were randomized 1:1 to receive 1 of 2 sequences consisting of 3 periods with up to 72 hours of washout between treatments. The first 2 periods were dosed under fasted state and the third period was dosed in a fed state (high-fat meal).

Sequence 1 began with powder-in-capsule formulation in Period 1 (fasted), followed by tablet formulation in Period 2 (fasted) and tablet formulation in Period 3 (fed). Sequence 2 began with tablet formulation in Period 1 (fasted), followed by powder-in-capsule formulation in Period 2 (fasted) and tablet formulation in Period 3 (fed).

### 4.4 Part 4: Narcolepsy Type 1 (NT1)

Part 4 of the study explored proof of concept (POC) in subjects with narcolepsy type-1 (NT1).

Part 4 consisted of a 4-way, 4-period crossover study design in approximately 8 subjects (2 subjects per sequence; minimum of 4 subjects with 1 subject per sequence). Subjects were randomized 1:1:1:1 to 1 of 4 sequences with 4 treatment periods (3 single doses of Compound A and 1 dose of matching placebo, with up to 48 hours of washout between treatments. Treatments were administered under fasted state. Dose levels were as follows:

| Part No. and | Cohort | | |
|---|---|---|---|
| Description | No. | Dose Level | Associated Study Drugs |
| Part 4 NT1 | NA | 1, 3, and 8 mg | Powder-in-capsule and matching |
| | | | placebo |

### 4.5 Part 5: Narcolepsy Type 2 (NT2)

Part 5 of the study explored proof of concept (POC) in subjects with narcolepsy type-2 (NT2).

Part 5 consisted of a 4-way, 4-period crossover study design in approximately 8 subjects (2 subjects per sequence; minimum of 4 subjects with 1 subject per sequence). Subjects were randomized 1:1:1:1 to 1 of 4 sequences with 4 treatment periods (3 single doses of Compound A and 1 dose of matching placebo), with up to 48 hours of washout between treatments. Treatments were administered under fasted state. Dose levels were as follows:

| Part No. and | Cohort | | |
|---|---|---|---|
| Description | No. | Dose Level | Associated Study Drugs |
| Part 5 NT2 | NA | 5, 12, and 25 mg | Powder-in-capsule and |
| | | | matching placebo |

### 4.6 Part 6: Idiopathic Hypersomnia (IH)

Part 6 of the study explored proof of concept (POC) in subjects with idiopathic hypersomnia (IH).

Part 6 consisted of a 4-way, 4-period crossover study design in approximately 8 subjects (2 subjects per sequence; minimum of 4 subjects with 1 subject per sequence). Subjects were randomized 1:1:1:1 to 1 of 4 sequences with 4 treatment periods (3 single doses of Compound A and 1 dose of matching placebo), with up to 48 hours of washout between treatments. Treatments were administered under fasted state. Dose levels were as follows:

| Part No. and | Cohort | | |
|---|---|---|---|
| Description | No. | Dose Level | Associated Study Drugs |
| Part 6 IH | NA | 5, 12, and 25 mg | Powder-in-capsule and |
| | | | matching placebo |

### 4.7 Study Periods

There were up to 3 study visits for all study parts (Screening, Treatment, and Safety Follow-up/End of Study [EOS]/Early Termination [ET]).

### 4.7.1 Part 1

For Part 1 (SAD), the Screening Visit (Visit 1) occurred between Day −28 and Day −3. During the Screening Visit, the subject was provided informed consent, followed by review of eligibility criteria, medical history, and physical examination.

### 4.7.2 Part 2

For Part 2 (MAD), the Screening Visit (Visit 1) occurred between Day −28 and Day −2. During the Screening Visit, the subject was provided informed consent, followed by review of eligibility criteria, medical history, and physical examination.

### 4.7.3 Part 3

For Part 3 (RelBA with FE), the Screening Visit (Visit 1) occurred between Day −28 and Day −2. During the Screening Visit, the subject was provided informed consent, followed by review of eligibility criteria, medical history, and physical examination.

### 4.7.4 Part 4

For Part 4, the Screening Visit (Visit 1) occurred between Day −28 and Day −3. During the Screening Visit, the subject was provided informed consent, followed by review of eligibility criteria, medical history, concomitant medication, and physical examination. Subjects underwent a washout from their current prescription medications that could interfere with the study (including medications used for the treatment of narcolepsy and cataplexy, as applicable). The washout period was at least 14 days (or 5 half-lives, whichever was longer) prior to Visit 2 and continued until the subject's discontinuation from participation in this study or discharge from the in-clinic visit on Day 9 (whichever was earlier).

### 4.7.5 Part 5

For Part 5, the Screening Visit (Visit 1) occurred between Day −28 and Day −3. During the Screening Visit, the subjects provided informed consent, followed by review of eligibility criteria, medical history, concomitant medication, and physical examination. Subjects underwent a washout from their current prescription medications that could interfere with the study (including medications used for the treatment of narcolepsy, as applicable). The washout period is at least 14 days (or 5 half-lives, whichever is longer) prior to Visit 2.

### 4.7.6 Part 6

For Part 6, the Screening Visit (Visit 1) occurred between Day −28 and Day −3. During the Screening Visit, the subjects provided informed consent, followed by review of eligibility criteria, medical history, concomitant medication, and physical examination. Subjects underwent a washout from their current prescription medications that could interfere with the study (including medications used for the treatment of idiopathic hypersomnia, as applicable). The washout period is at least 14 days (or 5 half-lives, whichever is longer) prior to Visit 2.

### 4.8 Treatment Visits

### 4.8.1 Part 1

The Treatment Visit (Visit 2) began on Day −2, when each subject's eligibility criteria were reviewed, and eligible subjects were admitted to the clinic. Screening assessments and eligibility criteria review continued after admission until Day 1, prior to randomization and dosing.

On Day −1, approximately 25 hours prior to study drug administration (on Day 1), baseline assessments for qEEG began and were collected for approximately 12 hours.

On Day 1, subjects were administered a single dose of either Compound A or matching placebo as per the randomization schedule with approximately 240 mL of noncarbonated water following overnight fasting. No food was given for at least 4 hours after dose administration. Water was allowed ad libitum except for the time 1 hour before and 1 hour after study drug administration. Safety assessments including AEs, safety ECGs, vital signs, and clinical laboratory tests were conducted, and plasma PK samples were collected. Continuous measurements with Holter ECG began approximately 1 hour prior to study drug administration on Day 1 until 24 hours post-dose. In addition, qEEG was monitored pre-dose and for 12 hours post-dose. Furthermore, BP and HR were monitored once every hour for 12 hours.

Subjects stayed at the clinic until approximately 48 hours after study drug administration and were discharged from the clinic on Day 3. Overall, subjects participating in Part 1 of the study had 4 overnight stays at the clinical site.

### 4.8.2 Part 2

The Treatment Visit (Visit 2) began on Day −1, when each subject's eligibility criteria were reviewed, and eligible subjects were admitted to the clinic. Screening assessments and eligibility criteria review continued after admission until Day 1, prior to randomization and dosing.

On Day 1, subjects in Cohorts 1-4 were administered a single dose of either Compound A or matching placebo as per the randomization schedule; subjects in Cohort 5 (open label) were administered a single dose of Compound A. All doses were administered with approximately 240 mL of noncarbonated water following overnight fasting. No food was given for at least 4 hours after dose administration. Water was allowed ad libitum except for the time 1 hour before and 1 hour after study drug administration. Safety assessments including AEs, safety ECGs, vital signs, and clinical laboratory tests were conducted, and plasma PK and urine samples were collected.

From Day 2 through Day 8, subjects were administered a single dose of either Compound A or matching placebo in the same manner as on Day 1. Safety assessments were made. A plasma PK sample was collected pre-dose on Day 8.

On Day 9, subjects were administered a single dose of either Compound A or matching placebo in the same manner as on Day 1. Safety assessments including AEs, safety ECGs, BP, and HR monitoring were conducted. A plasma PK sample was collected pre-dose on Day 9. For Cohorts 1-4, urine samples were collected for urine PK evaluation.

On Day 10, subjects were administered a single dose of either Compound A or matching placebo in the same manner as on day 1. Safety assessments, including AEs, safety ECGs, vital signs, and clinical laboratory tests, were conducted, and PK samples were collected. For Cohorts 1-4, continuous measurements with Holter ECG began approximately 1 hour prior to study drug administration on Day 10 until 24 hours post-dose.

Subjects stayed at the clinic until approximately 48 hours after the last administration of study drug on Day 10 and were discharged from the clinic on Day 12. Overall, subjects participating in Part 2 of the study had 13 days of overnight stay at the clinical site.

### 4.8.3 Part 3

The Treatment Visit (Visit 2) will begin on Day −1, when each subject's eligibility criteria were reviewed, and eligible subjects were admitted to the clinic. All subjects were randomized to 2 sequences of 3 treatment periods. While Treatment Period 1 and Treatment Period 2 were administered under fasted conditions, Treatment Period 3 was administered under fed conditions.

In each period, subjects were administered a single dose of Compound A as a tablet formulation or powder-in-capsule formulation as per the randomization schedule with approximately 240 mL of noncarbonated water. In periods under the fasted condition, subjects fasted overnight prior to dosing, and no food was given for at least 4 hours after dose administration. In treatment period(s) under the fed condition, following overnight fast, all subjects were given a high-fat breakfast. Within 30 minutes of starting the consumption of the breakfast, the assigned treatment was administered. Following administration of study drug, safety assessments including AEs, safety ECGs, vital signs and clinical laboratory tests were conducted, and PK samples for the measurement of Compound A were collected in each treatment period. There was a 72-hour washout period between the treatments.

Subjects stayed at the clinic until approximately 48 hours after the last administration of study drug on Day 7 and were discharged from the clinic on Day 9. Overall, subjects participating in Part 3 of the study had 10 days of overnight stay at the clinical site.

### 4.8.4 Part 4

The Treatment Visit (Visit 2) began on Day −2, when each subject's eligibility criteria were reviewed, and eligible subjects were admitted to the clinic. Screening assessments and eligibility criteria review continued after admission until Day 1, prior to randomization and dosing.

On Day −1, approximately 25 hours prior to study drug administration (on Day 1), baseline assessments for Maintenance of Wakefulness Test (MWT) began and were collected in a series of trials over approximately 10 hours.

Subjects were randomized to 1 of 4 sequences. Each sequence had 4 treatment periods (3 single-dose levels of Compound A and 1 dose of placebo). In each period, subjects were administered a single dose of study drug (either Compound A or placebo) as per the randomization schedule with approximately 240 mL of noncarbonated water following overnight fasting. No food was given for at least 3 hours after dose administration (i.e., fast can be broken after the 3-hour PK time point was collected). Water was allowed ad libitum except for the time 1 hour before and 1 hour after study drug administration. Safety assessments including AEs, safety ECGs, vital signs, and clinical laboratory tests were conducted, and plasma PK samples were collected. Furthermore, BP and HR were monitored once every hour for 12 hours.

There was a 48-hour washout period between the treatment periods.

Subjects stayed at the clinic until approximately 48 hours after the last administration of study drug on Day 7 and were discharged from the clinic thereafter. Overall, subjects participating in Part 4 of the study had 10 overnight stays at the clinical site.

### 4.8.5 Part 5

The Treatment Visit (Visit 2) began on Day −2, when each subject's eligibility criteria was reviewed, and eligible subjects were admitted to the clinic. Screening assessments and eligibility criteria review continued after admission until Day 1, prior to randomization and dosing.

On Day −1, approximately 25 hours prior to study drug administration (on Day 1), baseline assessments for Maintenance of Wakefulness Test (MWT) began and were collected in a series of trials over approximately 10 hours.

Subjects were randomized to 1 of 4 sequences. Each sequence had 4 treatment periods (3 single-dose levels of Compound A and 1 dose of placebo). In each period, subjects were administered a single dose of study drug (either Compound A or placebo) as per the randomization schedule with approximately 240 mL of noncarbonated water following overnight fasting. No food was given for at least 3 hours after dose administration (i.e., fast can be broken after the 3-hour PK time point is collected). Water was allowed ad libitum except for 1 hour before and 1 hour after study drug administration. Safety assessments including AEs, safety ECGs, vital signs, and clinical laboratory tests were conducted, and plasma PK samples were collected. Furthermore, BP and HR were monitored once every hour for 12 hours on the dosing days.

There was a 48-hour washout period between the treatment periods.

Subjects stayed at the clinic until approximately 48 hours after the last administration of study drug on Day 7 and will be discharged from the clinic thereafter. Overall, subjects participating in Part 5 of the study had 10 overnight stays at the clinical site.

### 4.8.6 Part 6

The Treatment Visit (Visit 2) began on Day −2, when each subject's eligibility criteria were reviewed, and eligible subjects were admitted to the clinic. Screening assessments and eligibility criteria review continued after admission until Day 1, prior to randomization and dosing. On Day −1, approximately 25 hours prior to study drug administration (on Day 1), baseline assessments for Maintenance of Wakefulness Test (MWT) began and were collected in a series of trials over approximately 10 hours.

Subjects were randomized to 1 of 4 sequences. Each sequence had 4 treatment periods (3 single-dose levels of Compound A and 1 dose of placebo). In each period, subjects were administered a single dose of study drug (either Compound A or placebo) as per the randomization schedule with approximately 240 mL of noncarbonated water following overnight fasting. No food was given for at least 3 hours after dose administration (i.e., fast can be broken after the 3-hour PK time point is collected). Water was allowed ad libitum except for 1 hour before and 1 hour after study drug administration. Safety assessments including AEs, safety ECGs, vital signs, and clinical laboratory tests were conducted, and plasma PK samples were collected. Furthermore, BP and HR were monitored once every hour for 12 hours on the dosing days.

There was a 48-hour washout period between the treatment periods.

Subjects stayed at the clinic until approximately 48 hours after the last administration of study drug on Day 7 and were discharged from the clinic thereafter. Overall, subjects participating in Part 6 of the study had 10 overnight stays at the clinical site.

### 4.9 Time Windows

Unless otherwise indicated, assessments made relative to dosing (at time point 0 hours) adhered to the windows listed below. Time points indicate the beginning of the respective assessment (eg, qEEG, MWT) and the exact time of assessment was recorded (Table 1).

TABLE 1

| Assessment Time Point (Hours) | Window (Minutes) |
|---|---|
| −1 (Pre-dose) | ±20 |
| 0 (Dosing) | NA |
| 0.5 | ±2 |
| 1 through 11 | ±15 |
| 12 through 48 | ±20 |

### 4.10 Clinical Assessments

### 4.10.1 Appropriateness of Measurements

The MWT is a validated objective measure of the ability to stay awake for a defined period of time. The Epworth Sleepiness Scale (ESS) is a validated measure with high specificity and sensitivity for assessing subjective sleepiness. The Narcolepsy Severity Scale (NSS) and Idiopathic Hypersomnia Severity Scale (IHSS) have been validated in their respective patient population and assess the presence and severity of commonly reported symptoms. Additionally, the questionnaires selected for measuring quality of life (QoL), fatigue and cognitive function have been used in clinical trials, and some have been validated for use in sleep disorders, such as narcolepsy. The use of vital signs, including BP assessment, and standard AE reporting are appropriate since they are routinely used to assess the safety profile of drugs in clinical studies. The Columbia-Suicide Severity Rating Scale (C-SSRS) is able to determine clinically meaningful points at which a person may be at risk for an impending suicide attempt.

### 4.10.2 Sleep, Cataplexy, and Activity Assessments

The following sleep assessments were performed for the study parts indicated at specified time points.

### 4.10.2.1 Sleep Diary

At specified time points (Part 4), subjects completed a sleep diary, which included the time to bed, estimated time of sleep onset, time(s) and reason(s) out of bed overnight, time(s) of wakening, and duration of time out of bed.

At specified time points (Parts 5 and 6), subjects completed a sleep diary, which included the time to bed, estimated time of sleep onset, time(s) and reason(s) out of bed overnight, time(s) of wakening, and duration of time out of bed.

### 4.10.2.2 Cataplexy Diary

At specified time points (Part 4 only), subjects with NT1 completed a cataplexy diary, which included the approximate time of any cataplexy events.

### 4.10.2.3 Actigraphy

At specified time points (Part 4), actigraphy was measured using a wearable wrist device to capture approximate total sleep time, as well as activity level, frequency, and duration. Using actigraphy in combination with a sleep diary for at least 7 days prior to a baseline MWT is considered an acceptable alternative to conducting nocturnal PSG for measuring total sleep time.

At specified time points (Parts 5 and 6), actigraphy was measured using a wearable wrist device to capture approximate total sleep time, as well as activity level, frequency, and duration. Using actigraphy in combination with a sleep diary for at least 7 days prior to a baseline MWT is considered an acceptable alternative to conducting nocturnal PSG for measuring total sleep time.

4.10.2.4 Maintenance of Wakefulness Test

At specified time points (Part 4), MWT assessments were performed and took up to 40 minutes to complete. The MWT is the standard objective measure of an individual's ability to remain awake during the daytime in a darkened, quiet environment and is commonly used to assess response to treatment. The MWT assessments were performed according to American Academy of Sleep Medicine (AASM) guidance. During the MWT trials, subjects were seated in a bed or reclining chair in a darkened room with their back and head supported such that their neck is not uncomfortably flexed or extended. Subjects are instructed to sit still and remain awake for as long as possible during each 40-minute trial. If the subject fell asleep during a trial, they were awakened, and the trial was terminated. If the subject did not fall asleep, then the trial was terminated at 40 minutes, and a sleep latency of 40 minutes was recorded. Upon completion of the trial, the subject returned to a normal environment/ambiance and remained awake (and were awakened if they fall asleep) until the next trial.

At specified time points (Parts 5 and 6), MWT assessments were performed and took up to 40 minutes to complete. During the MWT trials, subjects were seated in a bed or reclining chair in a darkened room with their back and head supported such that their neck is not uncomfortably flexed or extended. Subjects were instructed to sit still and remain awake for as long as possible during each 40-minute trial. If the subject fell asleep during a trial, they were awakened, and the trial was terminated. If the subject did not fall asleep, then the trial was terminated at 40 minutes, and a sleep latency of 40 minutes was recorded. Upon completion of the trial, the subject returned to a normal environment/ambiance and remained awake (and were awakened if they fall asleep) until the next trial.

Once Part 4 was completed, the EEG data recorded during the MWT, as well as the technician notes, were transferred to the EEG vendor for further analysis. Technician notes were also maintained for transfer to Sponsor at the end of the study. A precise assessment of sleep latency was conducted by an experienced sleep scorer from the EEG vendor, and spectral analysis was performed on the EEG data recorded during MWT to assess absolute and relative powers in standard frequency bands (i.e., qEEG). All raw data and analysis results were transferred to the Sponsor.

Once Parts 5 and 6 were completed, the EEG data recorded during the MWT, as well as the technician notes, were transferred to the EEG vendor for further analysis. Technician notes were also maintained for transfer to Sponsor at the end of the study. A precise assessment of sleep latency was conducted by an experienced sleep scorer from the EEG vendor, and spectral analysis was performed on the EEG data recorded during MWT to assess absolute and relative powers in standard frequency bands (i.e., qEEG). All raw data and analysis results were transferred to the Sponsor.

### 4.10.3 Subject Questionnaires

The following subject questionnaires were performed for the study parts indicated at specified time points.

### 4.10.3.1 Epworth Sleepiness Scale (ESS)

At points within the study, subjects were asked to complete the ESS with regard to the level of sleepiness they experienced over the past 7 days.

The ESS is well established as a subjective, self-reported outcome measure of EDS severity across multiple conditions. The ESS provides an assessment of a person's general level of daytime sleepiness, or average sleep propensity in daily life. The ESS is a validated measure with high specificity and sensitivity for assessing subjective sleepiness. The ESS is comprised of 8 items asking subjects how likely they are to doze off or fall asleep in different situations. Responses to each item range from 0=would never doze to 3=high chance of dozing, and responses are summed to a total score (0 to 24). ESS scores can also be categorized as follows: 0-5 (lower normal daytime sleepiness), 6-10 (higher normal daytime sleepiness), 11-12 (mild EDS), 13-15 (moderate EDS), and 16-24 (severe EDS).

### 4.10.3.2 Karolinska Sleepiness Scale (KSS)

At points within the study, subjects were asked to rate their sleepiness using the KSS with regard to the level of sleepiness they experienced over the past 5 minutes.

The KSS is a validated, self-reported outcome measure of subjective sleepiness. The KSS was developed as a one-dimensional measure of sleepiness and has been validated against assessments like EEG and electrooculography activity (Akerstedt and Gillberg 1990). The KSS is comprised of a single question that asks the subject to rate their sleepiness in the immediately preceding 5 minutes on a 9-point scale where 1=extremely alert and 9=very sleepy, great effort to keep awake, fighting sleep. The KSS takes approximately 10 to 15 seconds to complete. Thus, the KSS is well suited for repeated measurement at short intervals as it assesses situational sleepiness within a short timeframe.

4.10.3.3 Narcolepsy Severity Scale (Subjects with NT1 or NT2 Only)

At points within the study, subjects who have been diagnosed with NT1 or NT2 will complete the NSS. The NSS is a 15-item self-reported outcome measure validated for use in NT1 patients. It evaluates the severity, frequency, and impact of the 5 main narcolepsy symptoms (excessive daytime sleepiness (EDS), cataplexy, hallucinations, sleep paralysis, and disrupted nighttime sleep (DNS)), with higher scores indicating more severe symptoms. Except for cataplexy, these narcolepsy symptoms are also relevant for some NT2 patients, and thus, we will use the NSS in both NT1 and NT2 patients.

### 4.10.3.4 Idiopathic Hypersomnia (IH) Severity Scale (Subjects With IH Only)

At points within the study, subjects who have been diagnosed with IH will complete the IHSS. The IHSS is a self-reported measure validated in patients with IH. It is a 14-item questionnaire that measures aspects of nighttime and daytime sleep symptoms and the sleep inertia related to each, as well as impaired daytime functioning due to hypersomnolence. Symptom frequency, intensity, and consequences are rated using a 3- or 4-point scale, with higher scores indicating more severe and frequent symptoms.

4.10.3.5 Weekly Cataplexy Rate (Subjects with NT1 Only)

At points within the study, subjects who have been diagnosed with NT1 will be asked how many cataplectic attacks they have in an average week (i.e., WCR).

### 4.10.3.6 Quality of Life and Severity of Illness

In Part 4, questionnaires aimed at assessing QOL, fatigue or cognitive function in study subjects with NT1, NT2, and IH included the Functional Outcomes of Sleep Questionnaire (FOSQ) and the Clinical Global Impression of Severity (CGI-S). The FOSQ and the CGI-S may be included in Parts 5 and 6.

The FOSQ is a 30-item disease specific QOL questionnaire to determine functional status in adults; measures are designed to assess the impact of disorders of excessive sleepiness on multiple activities of everyday living and the extent to which these activities are improved by effective treatment. The FOSQ-10 is a short version of the original 30-item FOSQ that has been shown to perform similarly to the longer version. The FOSQ-10 has been shown to exhibit high internal consistency, and effect sizes and pre- and post-treatment differences that are highly correlated with the longer version.

The CGI-S is a 7-point Likert-type rating scale and a widely used assessment in clinical psychopharmacology trials to assess severity of illness. The responses of this Investigator-completed scale range from 1=normal, no signs of illness to 7=among the most extremely ill patients. The Investigator will rate their impression of the severity of the subject's current condition at Baseline relative to their experience with this patient population.

### 4.11 Safety Assessments

### 4.11.1 Adverse Event Monitoring and Reporting

Adverse events (AEs) and serious adverse events (SAEs) were monitored continuously until the completion of the study.

### 4.11.2 Physical Examination

At points within the study, physical examinations were performed and included evaluation of the head, eyes, ears, nose, throat, neck, chest, lungs, abdomen, extremities, skin, and neurological examination during the Screening period. Height and weight were also measured and recorded.

4.11.3 Hematology, Biochemistry, Urinalysis and Drug testing

At points within the study, blood and urine samples for laboratory assessments were collected. Samples were collected in accordance with the site's usual procedures and analyzed by the site's local laboratory.

An alcohol breath test and urine drug screen were also performed. A repeat urine test for drugs of abuse may be performed to assess for a possible false test result.

### 4.11.4 Vital Signs and Electrocardiograms

The subject should be resting in a supine position for at least 5 minutes before assessments are measured.

### 4.11.5 Safety Electrocardiogram (ECG)

At points within the study, safety ECGs were obtained using an ECG machine that automatically calculates the HR and measures PR, QRS, QT, QTcF, and QTcB intervals. The subject remained supine for the entire assessment. Safety ECGs can be extracted from the Holter device.

### 4.11.6 Respiratory Rate and Body Temperature

Respiratory rate and body temperature were assessed. The subject remained supine for the entire assessment.

### 4.11.7 Blood Pressure (BP) and Heart Rate (HR) Monitoring

BP (including systolic BP and diastolic BP) and HR assessments were performed using an automatic BP. The subject remained supine for the entire assessment.

### 4.11.8 Holter Monitoring

Continuous ECG recording by Holter monitoring device was conducted.

### 4.11.9 Suicidal Ideation and Behavior Risk Monitoring

Assessment of suicidal ideation was conducted using the C-SSRS. The “Screening/Baseline” C-SSRS was administered at the Screening Visit; “Since Last Visit” C-SSRS was administered at all subsequent time points. Any signs of suicidal ideation or behavior are considered an AE. Subjects who exhibit or report signs of suicidal ideation or behavior will undergo a risk assessment and are considered for discontinuing participation in the study in addition to appropriate referral for follow-up based on the judgment of the Investigator.

### 4.12 Genetic Assessments

### 4.12.1 Pharmacogenetic Assessment

On Day 1, after randomization, a whole blood sample (up to 3 mL per sample) for pharmacogenetic analysis was collected. DNA extracted from the blood sample was analyzed for variants in the gene(s) responsible for drug absorption, distribution, metabolism, or excretion, including the CYP3A4 gene. CYP3A4 is the enzyme primarily responsible for metabolizing Compound A, and genetic variation in the CYP3A4 gene is known to affect the function of the CYP3A4 enzyme.

### 4.12.2 Human Leukocyte Antigen (HLA) Assessment

On Day 1, after randomization, a whole blood sample (up to 3 mL per sample) for HLA analysis was collected. DNA extracted from the blood sample was analyzed for variants in the HLA complex family of genes to determine presence or absence of genetic alleles associated with risk for narcolepsy, including HLA-DQB1 and HLA-DRB1.

### 4.13 Pharmacokinetic and Pharmacodynamic Assessments

### 4.13.1 Pharmacokinetic Sample Collection

### 4.13.1.1 Plasma PK

Blood samples (up to 5 mL per sample) were collected for PK analysis of Compound A and its metabolite(s). The actual time of sample collection was recorded. The PK samples of the subjects randomized to placebo treatment were not analyzed. Any remaining plasma PK samples were used to explore the presence of any additional metabolites and/or biomarkers.

### 4.13.1.2 Urine PK

Urine samples were collected for PK analysis of Compound A and its metabolite(s). Urine samples for subjects randomized to placebo were not analyzed.

### 4.13.2 Bioanalytical Methods

Plasma and urine concentrations of Compound A and its metabolite(s) were measured using a validated high-performance liquid chromatography with tandem mass spectrometry.

### 4.13.3 Pharmacokinetic Parameters

The plasma PK parameters of Compound A and its metabolite(s) were determined from the plasma concentration-time profiles for all evaluable subjects. Actual sampling times, rather than scheduled sampling times, were used in all computations involving sampling times. The below lower limit of quantitation concentrations were set to zero prior to PK parameter estimation.

### 4.13.4 EEG Biomarkers

At time points within Part 1 of the study, qEEG assessment was performed. The subject remained still in a seated or supine position during the EEG acquisition time points. Each EEG acquisition continued for a total of 10 minutes: initial 5 minutes with eyes closed followed by 5 minutes with eyes open looking at a fixation point.

In Part 4, qEEG parameters were extracted from EEG recordings collected during the MWT trials; event-related potential (ERP) parameters were extracted from EEG recordings collected during the SART trials.

In Parts 5 and 6, qEEG parameters were extracted from EEG recordings collected during the MWT trials; ERP parameters were extracted from EEG recordings collected during the SART trials.

### 4.13.5 Sustained Attention to Response Task (SART)

At the time points, the computer-based SART assessments were performed, in which subjects were instructed to push a button in response to viewing select visual stimuli (and not others). EEG-based ERPs associated with the SART were explored. Relevant peaks' amplitude and latency were captured.

Cognitive function is one of the most important QOL factors for narcolepsy patients. Sustained attention is the domain of cognitive function most consistently observed as impaired in patients with narcolepsy and is required for many activities of daily living, like driving and work productivity. SART is a behavioral task that yields multiple quantitative measures based on the number and type of errors a patient makes. Narcolepsy patients have significantly higher error rates on the SART, indicative of substantial impairment in sustained attention and vigilance.

### 4.14.1 Analysis of PK Data

Pharmacokinetic analyses were based on the PK population. Plasma and urine concentrations of Compound A and its metabolite(s) (if measured) were analyzed by noncompartmental methods to determine their PK parameters.

### 4.14.1.1 Part 1 (SAD)

The PK population was used for summaries of plasma Compound A and its metabolite(s) concentrations and for analyses, summaries, and listing of PK parameters. Dose proportionality for Cₘₐₓ, AUC₀₋ₜ, and AUC_{0-∞} was evaluated using a linear model of logarithmically transformed values to assess the slope parameter, and the 95% CI for the slope was estimated. Where the 95% CI of the slope includes 1, it was considered as evidence of dose proportionality for that PK parameter.

### 4.14.1.2 Part 2 (MAD)

The PK population was used for summaries of plasma and urine Compound A and its metabolite(s) concentrations and for analyses, summaries, and listing of PK parameters. Accumulation ratio (Day 10/Day 1) for Cₘₐₓ and AUC₀₋₂₄ was estimated with 90% CIs using a linear fixed effects model on log-transformed parameters. Dose effect was assessed using a linear fixed effects model on log-transformed t_{1/2}. Dose proportionality was assessed using a power model on Cₘₐₓ and AUC₀₋₂₄ on Day 1 and Day 10 separately. Within-subject and total standard deviations for log-transformed Cₘₐₓ and AUC₀₋₂₄ were estimated within a linear mixed effects framework.

4.14.1.3 Part 3 (RelBA with FE)

The PK population were used for summaries of plasma Compound A concentrations and for analyses, summaries, and listing of PK parameters.

Relative bioavailability analysis: A mixed effects model was used to evaluate the effect of a tablet formulation on the PK of Compound A. The primary PK parameters, namely, Cₘₐₓ, AUC₀₋ₜ, and AUC₀%, were logarithmically transformed and used as the dependent variable, with treatment (tablet formulation or powder-in-capsule) as a fixed effect, and subject as a random effect. The ratio of the geometric means of the 2 treatments was obtained from the analysis, and a 2-sided 90% CI for the ratio was estimated. In this comparison, the same dose level of Compound A administered under tablet formulation is the test and powder-in-capsule is the reference. Thus, the comparison of interest is as follows: Fasted in new formulation (Test) vs Fasted in powder-in-capsule (Reference).

Food effect analysis: A mixed effects model was used to evaluate the effect of a high-fat, high caloric meal on the PK of Compound A in the tablet formulation. The primary PK parameters, namely, Cₘₐₓ, AUC₀₋ₜ, and AUC_{0-∞}, were logarithmically transformed and used as the dependent variable, with treatment (fed or fasted) as a fixed effect, and subject as a random effect. The ratio of the geometric means of the 2 treatments was obtained from the analysis, and a 2-sided 90% CI for the ratio was estimated. In this comparison, the same dose level of Compound A administered under fed conditions is the test and Compound A administered under fasted conditions is the reference. Thus, the comparison of interest is as follows: Fed a high-fat meal in tablet formulation (Test) vs Fasted in tablet formulation (Reference).

### 4.14.1.4 Part 4

The PK population was used for summaries of plasma Compound A and its metabolite(s) concentrations and for analyses, summaries, and listing of PK parameters. Dose proportionality for Cₘₐₓ, AUC₀₋ₜ, and AUC_{0-∞} was evaluated using a linear model of logarithmically transformed values to assess the slope parameter, and the 95% CI for the slope was estimated. Where a 95% CI of the slope includes 1, it was considered as evidence of dose proportionality for that PK parameter.

### 4.14.1.5 Part 5

The PK population was used for summaries of plasma Compound A and its metabolite(s) concentrations and for analyses, summaries, and listing of PK parameters. Dose proportionality for Cₘₐₓ, AUC₀₋ₜ, and AUC_{0-∞} was evaluated using a linear model of logarithmically transformed values to assess the slope parameter, and the 95% CI for the slope was estimated. Where a 95% CI of the slope includes 1, it was considered as evidence of dose proportionality for that PK parameter.

### 4.14.1.6 Part 6

The PK population was used for summaries of plasma Compound A and its metabolite(s) concentrations and for analyses, summaries, and listing of PK parameters. Dose proportionality for Cₘₐₓ, AUC₀₋ₜ, and AUC_{0-∞} was evaluated using a linear model of logarithmically transformed values to assess the slope parameter, and the 95% CI for the slope was estimated. Where a 95% CI of the slope includes 1, it was considered as evidence of dose proportionality for that PK parameter.

### 4.15 Analysis of PD/Efficacy Data

### 4.15.1 Part 1 (SAD)

The PD parameters in the SAD evaluation (e.g., changes from baseline in QTcF, power of qEEG frequency bands, KSS) were summarized using descriptive statistics for each time point by dose level.

### 4.15.2 Part 2 (MAD)

The PD parameters in the MAD evaluation (e.g., changes from baseline in KSS, BP) were summarized using descriptive statistics for each time point by visit (e.g., Day 1, Day 10) and by dose level.

### 4.15.3 Part 4

The PD parameters in the POC evaluation (e.g., changes from baseline in sleep latency on MWT, number of errors on SART and level of sleepiness on KSS at matched time points) were summarized using descriptive statistics for each time point by dose level.

For the changes from baseline in average sleep latency across the first 4 sessions of the MWT, descriptive statistics were provided by dose level. Mixed model with repeated measure (MMRM) for the 4-period crossover design were conducted with the change from baseline as a dependent variable, and the dose level and the period as fixed factors. The differences in the least square means between each Compound A dose level and the placebo (each Compound A dose level—the placebo), and the 2-sided CIs were provided.

### 4.15.4 Part 5

The PD parameters in the POC evaluation (e.g., changes from baseline in sleep latency on MWT, number of errors on SART and level of sleepiness on KSS at matched time points) were summarized using descriptive statistics for each time point by dose level.

For the changes from baseline in average sleep latency across the first 4 sessions of the MWT, descriptive statistics were provided by dose level. Mixed model with repeated measure (MMRM) for the 4-period crossover design were conducted with the change from baseline as a dependent variable, and the dose level and the period as fixed factors. The differences in the least square means between each Compound A dose level and the placebo (each Compound A dose level—the placebo), and the 2-sided CIs were provided.

### 4.15.5 Part 6

The PD parameters in the POC evaluation (e.g., changes from baseline in sleep latency on MWT, number of errors on SART and level of sleepiness on KSS at matched time points) were summarized using descriptive statistics for each time point by dose level.

For the changes from baseline in average sleep latency across the first 4 sessions of the MWT, descriptive statistics were provided by dose level. Mixed model with repeated measure (MMRM) for the 4-period crossover design were conducted with the change from baseline as a dependent variable, and the dose level and the period as fixed factors. The differences in the least square means between each Compound A dose level and the placebo (each Compound A dose level—the placebo), and the 2-sided CIs were provided.

### 4.16 Other Safety-PK-PD Analysis

### 4.16.1 Holter Electrocardiograms (Holter ECG)

Expert precision QT analyses (Conc-QTcF) were performed using data extracted from the 24-hour Holter recording obtained from baseline through 24 hours post-dose according to the procedures and algorithm of the ECG vendor. Alternate correction methods for QT or QT beat-to-beat intervals were explored if QTcF is deemed not to be appropriate. QT data was summarized by dose/time point and treatment group.

The relationship between Compound A plasma concentrations and QT interval was explored based on PK analysis dataset. Lack of drug effect on QTcF interval was concluded if the upper bound of 2-sided 90% CIs for the true mean difference in placebo-corrected QTcF change from baseline is less than 10 ms.

### 4.16.2 PK-PD Analysis

Relationship between Compound A dose and/or plasma concentration and PD parameters were conducted in graphical manner and where appropriate, PK-PD modeling was used to further evaluate the relationship.

### 4.16.3 PK-Safety Analysis

Where the data permits, the PK-safety relationship for any safety finding of interest was assessed by integrating data across the dose cohorts. If the 90% CI for the slope of the relationship between PK and the safety parameter includes zero, it was concluded that there is no effect within the dose range studied.

### 4.17 Overall Findings

### 4.17.1 Overall Safety (Parts 1-4)

Treatment with Compound A was well tolerated in all doses by healthy volunteers and patients with NT1. Most TEAEs were mild to moderate in severity. No deaths, serious, or severe TEAEs were reported. The most common drug related TEAEs (reported in more than 1 subject on active treatment) in healthy volunteers included:
in Part 1 (SAD): dizziness, pollakiuria, nausea, vision blurred
in Part 2 (MAD): insomnia, dizziness, pollakiuria, visual impairment (described as transitory episodes of light sensitivity and distorted vision)
in Part 3 (RelBA with FE, not placebo-controlled): dizziness, hypervigilance (described as feeling an increase of alertness and/or more energy), insomnia, headache, and pollakiuria.

The most common drug related TEAEs (reported in more than 1 subject) in NT1 patients included insomnia, salivary hypersecretion, and pollakiuria. There were no clinically meaningful drug-related changes from baseline of laboratory values, vital signs, or ECGs.

### 4.17.2 Overall PK

Compound A was well tolerated following single doses up to 50 mg and multiple doses of up to 25 mg after once daily administration for 10 days. Compound A was rapidly absorbed with a median time to achieve Cₘₐₓ (time at which the highest drug concentration occurs [Tₘₐₓ]) ranging from 1 to 2.5 hours in fasted subjects. Following administration of single doses of Compound A, geometric mean Cₘₐₓ was less than dose proportional from 1 to 50 mg (50-fold change in dose resulted into 9-fold change in Cₘₐₓ). Geometric mean AUCₗₐₛₜ increased approximately dose proportionally across all doses (50-fold change resulted into 29-fold change in AUC). Following daily administration of Compound A at 3, 8, 15 and 25 mg for 10 days, the accumulation ratio of Compound A was approximately 1.2 to 1.7 for Cₘₐₓ and approximately 1 to 1.4 for AUC₀₋₂₄. The half-life following multiple oral dosing of Compound A was approximately 8 to 16 hours. Administration of Compound A tablet in fed condition increased peak plasma concentration by 3-fold (Cₘₐₓ: 10.8 ng/ml in fasted vs. 30.1 ng/mL in fed condition) while having no impact on extent of absorption.

### 4.17.3 Overall Efficacy

Evaluation of Compound A effects on the change of average sleep latency across 4 timepoints of MWT (2-, 4-, 6-, and 8-hours post-dose) from baseline in 10 patients with NT1 showed statistically significant improvement in sleep latency in all doses tested (1, 3, and 8 mg) compared with placebo.

### 4.18.1 Part 1 (SAD)

In Part 1 (SAD), six dosing cohorts (1, 3, 8, 15, 25, and 50 mg) were completed. A total of 48 subjects were enrolled in Part 1 (SAD) with 36 subjects receiving a single oral dose of Compound A, and 12 subjects receiving a single oral dose of matching placebo. All subjects were healthy adult subjects with no clinically significant illness or abnormality.

### Safety

Treatment with Compound A was well tolerated in all doses of Part 1 (SAD). No deaths, serious AEs, or Aes leading to discontinuation occurred during this part of the study (Table 2). Most of the Aes were mild to moderate in severity. Of the 36 subjects receiving a single oral dose of Compound A (1, 3, 8, 15, 25, and 50 mg), 15 had Aes. Drug-related adverse events were observed only in higher doses (15, 25, and 50 mg) and were mostly mild (Table 2).

Four moderate TEAEs were reported (back pain, COVID-19, dermatitis contact and pollakiuria); only pollakiuria reported as related to study drug (occurred on 50 mg dose. TEAEs that occurred in more than 1 subject included dizziness, pollakiuria, COVID-19 (occurred in three subjects in 1 mg cohort), nausea, vision blurred (Table 3).

TABLE 2
Summary of Treatment-Emergent Adverse Events (Safety Population) Part 1 SAD

| | | Compound A | | | | | | |
|---|---|---|---|---|---|---|---|---|
| | Placebo | 1 mg | 3 mg | 8 mg | 15 mg | 25 mg | 50 mg | Total |
| Number of | (N = 12) | (N = 6) | (N = 6) | (N = 6) | (N = 6) | (N = 6) | (N = 6) | (N = 36) |
| Subjects | n (%) | n (%) | n (%) | n (%) | n (%) | n (%) | n (%) | n (%) |
| Any TEAE | 3 | 4 | 0 | 1 | 4 | 4 | 2 | 15 |
| | (25.0) | (66.7) | | (16.7) | (66.7) | (66.7) | (33.3) | (41.7) |
| TEAE by Highest | | | | | | | | |
| Severity^{a} | | | | | | | | |
| Mild | 2 | 1 | 0 | 1 | 4 | 4 | 1 | 11 |
| | (16.7) | (16.7) | | (16.7) | (66.7) | (66.7) | (16.7) | (30.6) |
| Moderate | 1 | 3 | 0 | 0 | 0 | 0 | 1 | 4 |
| | (8.3) | (50.0) | | | | | (16.7) | (11.1) |
| Severe | 0 | 0 | 0 | 0 | 0 | 0 | 0 | 0 |
| TEAE by Highest | | | | | | | | |
| Relationship^{a} | | | | | | | | |
| Not Related | 3 | 4 | 0 | 1 | 0 | 0 | 0 | 5 |
| | (25.0) | (66.7) | | (16.7) | | | | (13.9) |
| Related | 0 | 0 | 0 | 0 | 4 | 4 | 2 | 10 |
| | | | | | (66.7) | (66.7) | (33.3) | (27.8) |
| AE Leading to Study | 0 | 0 | 0 | 0 | 0 | 0 | 0 | 0 |
| Drug Discontinuation | | | | | | | | |
| Any SAEs | 0 | 0 | 0 | 0 | 0 | 0 | 0 | 0 |
| Death | 0 | 0 | 0 | 0 | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.
^{a}If a subject has multiple AEs, the highest severity is presented in summary by severity, and the closest relationship to study drug is presented in summary by relationship.

TABLE 3
Summary of Treatment Emergent Adverse Events by
Preferred Term (Safety Population) Part 1 SAD

| | | Compound A | | | | | | |
|---|---|---|---|---|---|---|---|---|
| | Placebo | 1 mg | 3 mg | 8 mg | 15 mg | 25 mg | 50 mg | Total |
| | (N = 12) | (N = 6) | (N = 6) | (N = 6) | (N = 6) | (N = 6) | (N = 6) | (N = 36) |
| Preferred Term | n (%) | n (%) | n (%) | n (%) | n (%) | n (%) | n (%) | n (%) |
| Subjects with at | 3 | 4 | 0 | 1 | 4 | 4 | 2 | 15 |
| least one TEAE | (25.0) | (66.7) | | (16.7) | (66.7) | (66.7) | (33.3) | (41.7) |
| Dizziness | 0 | 0 | 0 | 0 | 2 | 4 | 2 | 8 |
| | | | | | (33.3) | (66.7) | (33.3) | (22.2) |
| Pollakiuria | 0 | 0 | 0 | 0 | 1 | 1 | 2 | 4 |
| | | | | | (16.7) | (16.7) | (33.3) | (11.1) |
| COVID-19 | 0 | 3 | 0 | 0 | 0 | 0 | 0 | 3 |
| | | (50.0) | | | | | | (8.3) |
| Nausea | 0 | 0 | 0 | 0 | 0 | 0 | 2 | 2 |
| | | | | | | | (33.3) | (5.6) |
| Vision blurred | 0 | 0 | 0 | 0 | 1 | 0 | 1 | 2 |
| | | | | | (16.7) | | (16.7) | (5.6) |
| Anxiety | 0 | 0 | 0 | 0 | 1 | 0 | 0 | 1 |
| | | | | | (16.7) | | | (2.8) |
| Aspartate | 0 | 0 | 0 | 0 | 0 | 0 | 1 | 1 |
| aminotransferase | | | | | | | (16.7) | (2.8) |
| increased | | | | | | | | |
| Blood pressure | 0 | 0 | 0 | 0 | 0 | 0 | 1 | 1 |
| increased | | | | | | | (16.7) | (2.8) |
| Dermatitis | 1 | 1 | 0 | 0 | 0 | 0 | 0 | 1 |
| contact | (8.3) | (16.7) | | | | | | (2.8) |
| Fatigue | 0 | 0 | 0 | 0 | 0 | 1 | 0 | 1 |
| | | | | | | (16.7) | | (2.8) |
| Headache | 1 | 0 | 0 | 0 | 0 | 0 | 1 | 1 |
| | (8.3) | | | | | | (16.7) | (2.8) |
| Hot flush | 0 | 0 | 0 | 0 | 1 | 0 | 0 | 1 |
| | | | | | (16.7) | | | (2.8) |
| Hypervigilance | 0 | 0 | 0 | 0 | 1 | 0 | 0 | 1 |
| | | | | | (16.7) | | | (2.8) |
| Insomnia | 0 | 0 | 0 | 0 | 1 | 0 | 0 | 1 |
| | | | | | (16.7) | | | (2.8) |
| Metamorphopsia | 0 | 0 | 0 | 0 | 1 | 0 | 0 | 1 |
| | | | | | (16.7) | | | (2.8) |
| Oropharyngeal | 0 | 0 | 0 | 0 | 0 | 1 | 0 | 1 |
| pain | | | | | | (16.7) | | (2.8) |
| Pharyngitis | 0 | 1 | 0 | 0 | 0 | 0 | 0 | 1 |
| | | (16.7) | | | | | | (2.8) |
| Skin | 0 | 1 | 0 | 0 | 0 | 0 | 0 | 1 |
| hyperpigmentation | | (16.7) | | | | | | (2.8) |
| Upper respiratory | 0 | 0 | 0 | 1 | 0 | 0 | 0 | 1 |
| tract infection | | | | (16.7) | | | | (2.8) |
| Urinary | 0 | 0 | 0 | 0 | 0 | 0 | 1 | 1 |
| incontinence | | | | | | | (16.7) | (2.8) |
| Back pain | 1 | 0 | 0 | 0 | 0 | 0 | 0 | 0 |
| | (8.3) | | | | | | | |

Abbreviations: TEAE = treatment emergent adverse event, N = number of subjects dosed with each drug (on Day 1); n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.

### Pharmacokinetics

The key clinical pharmacology findings following administration of single oral doses of Compound A (1, 3, 8, 15, 25, and 50 mg) were as follows. Compound A was rapidly absorbed with a median time to achieve Cₘₐₓ (time at which the highest drug concentration occurs [Tₘₐₓ]) ranging from 1 to 2.5 hours in fasted subjects. Following administration of single doses of Compound A, geometric mean Cₘₐₓ was less than dose proportional from 1 to 50 mg (50-fold change in dose resulted into 9-fold change in Cₘₐₓ). Geometric mean AUC increased approximately dose proportionally across all doses (50-fold change resulted into 29-fold change in AUC).

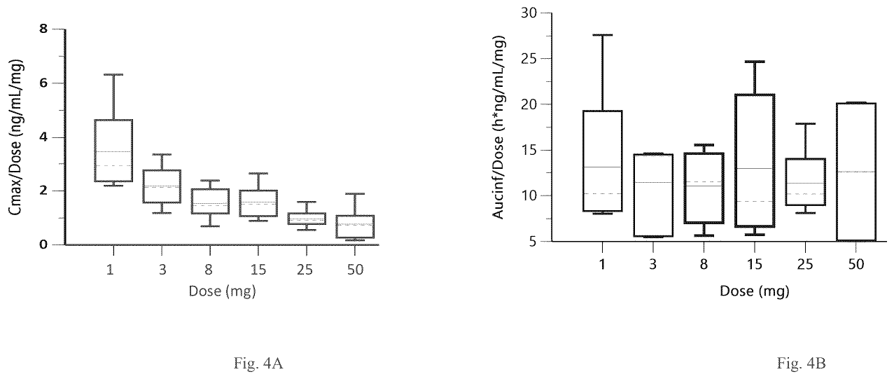

Mean Compound A plasma concentration-time profiles for all six cohorts are presented in FIG. 3. Following administration of single oral dose from 1 to 50 mg to healthy adult subjects, Compound A showed moderate absorption with a median Tₘₐₓ in the range of 1 to 2.5 hours. Compound A showed an increase in Cₘₐₓ with increase in dose, but the increase was not dose proportional. AUC_{0-inf} increased dose proportionally with the increase in dose from 1 to 50 mg. The PK profile for Compound A showed biphasic distribution (FIG. 3, Table 4). Following single oral administration of Compound A in the dose range of 1 to 50 mg, it was observed that the Cₘₐₓ increase appeared to be less than dose proportional (geometric mean Cₘₐₓ/D reduced from 3.2 to 0.59; FIG. 4A). AUC increase was generally dose proportional (geometric mean AUC_{0-inf}/D with minor change from 11.6 to 10.1; FIG. 4B).

The pharmacokinetic parameters of Compound A following administration of single oral doses are summarized in Table 4.

TABLE 4
Summary of Pharmacokinetic Parameters of Compound A Following
Single Oral Dose of Compound A - Part 1(SAD)

| | Compound A Dose | | | | | |
|---|---|---|---|---|---|---|
| | 1 mg | 3 mg | 8 mg | 15 mg | 25 mg | 50 mg |
| | N = 6 | N = 6 | N = 6 | N = 6 | N = 6 | N = 6 |
| Cₘₐₓ | 3.23 | 6.15 | 11.5 | 22.1 | 23.1 | 29.3 |
| (ng/mL) | (42.1) | (39.1) | (45.5) | (41.5) | (36.1) | (104) |
| C₈ | 0.337 | 1.24 | 2.47 | 4.07 | 5.70 | 7.83 |
| (ng/mL) | (69.5) | (80.5) | (67.5) | (97.3) | (52.1) | (103) |
| AUCₗₐₛₜ | 12.3 | 36.6 | 81.3 | 165 | 241 | 357 |
| (ng · h/mL) | (59.1) | (55.8) | (40.6) | (61.3) | (25.9) | (73.0) |
| AUC_{0-∞} | 11.6 | 31.4 | 83.0 | 167 | 274 | 503 |
| (ng · h/mL) | (53.4)^{a} | (61.0)^{b} | (45.7)^{c} | (67.4)^{a} | (29.3) | (128)^{d} |
| Tₘₐₓ | 1.5 | 1.5 | 2.0 | 2.5 | 1.0 | 2.0 |
| (h) | (0.5-2.0) | (1.0-3.0) | (1.0-6.0) | (1.0-3.0) | (0.5-1.5) | (1.0-3.0) |
| t_{1/2} | 2.79 | 2.31 | 16.0 | 10.1 | 12.5 | 7.34 |
| (h) | (33.9)^{a} | (46.0)^{b} | (50.0)^{c} | (60.7)^{a} | (59.3) | (92.0)^{d} |
| CL/F | 86.0 | 95.6 | 96.4 | 89.8 | 91.1 | 99.3 |
| (L/h) | (53.4)^{a} | (61.0)^{b} | (45.7)^{c} | (67.4)^{a} | (29.3) | (128)^{d} |
| Vz/F | 346 | 318 | 2224 | 1305 | 1649 | 1052 |
| (L) | (48.5)^{a} | (64.5)^{b} | (60.1)^{c} | (124)^{a} | (46.1) | (467)^{d} |

Abbreviations: AUC = area under the concentration-time curve, C₈ = concentration at 8-hour post dose, CL/F = apparent total clearance following oral administration, Cₘₐₓ = maximum plasma concentration, PK = pharmacokinetics, SAD = single ascending dose, t_{1/2} = half-life, Tₘₐₓ = time to reach Cₘₐₓ, Vz/F = apparent volume of distribution at terminal phase, CV % = coefficient of variation percentage.
^{a}N = 5 since 1 subject had unaccepted R2 < 0.8.
^{b}N = 3 since 3 subjects had unaccepted R2 < 0.8.
^{c}N = 4 since 2 subjects had unaccepted R2 < 0.8.
^{d}N = 2 since 4 subjects had unaccepted R2 < 0.8.
Data represented as geometric mean (geometric CV %). Tₘₐₓ is represented as median (range).

### 4.18.2 Part 2 (MAD)

In Part 2 (MAD), four dosing cohorts (3, 8, 15, and 25 mg) were completed. A total of 32 subjects were enrolled in Part 2 (MAD) with 23 subjects receiving multiple oral doses of Compound A, 8 subjects receiving multiple oral doses of matching placebo, and 1 subject who discontinued after receiving a single 25 mg dose of Compound A. All subjects were healthy adult subjects with no clinically significant illness or abnormality.

### Safety

Treatment with Compound A was well tolerated in all doses of Part 2 (MAD). No deaths or serious AEs occurred during this part of the study (Table 5). One subject discontinued the study drug and withdrew from study in Part 2 (MAD) due to AEs (Table 5). Most of the reported AEs were mild to moderate in severity (Table 5). Of the 24 subjects receiving multiple oral doses of Compound A (3, 8, 15, and 25 mg), 15 had AEs. TEAEs that occurred in more than 1 subject included dizziness, insomnia, pollakiuria, visual impairment (reported and described as a visual disturbance with transitory episodes of light sensitivity and distorted vision), and diarrhea (Table 6). Drug related adverse events were observed in doses 8 mg and above. There were no clinically significant abnormalities or notable trends observed following study drug administration, in the safety variables of chemistry or hematology laboratory findings, vital signs, or ECG measurements.

One subject discontinued study drug due to mild to moderate AEs (moderate visual disturbance, urinary frequency and euphoric mood, mild hypersalivation, disequilibrium and insomnia) after receiving a single 25 mg dose of Compound A. The subject experienced visual disturbances, disequilibrium, hypersalivation, euphoric mood, and pollakiuria shortly after the first study drug administration. Her neurological examination, laboratory parameters, vital signs and ECGs were unremarkable. The visual disturbance, sensation of disequilibrium, hypersalivation and euphoric mood (described as excitable, ‘good mood’), resolved without sequelae within several hours after the dosing. She had insomnia and continued urinary frequency, that resolved on the following day. Participant requested no further dosing and remained at the Clinical Research Unit for an additional overnight stay. There were no additional AEs or abnormalities noted and she was released from the Clinic, entering the 7-day safety follow-up period with all AEs resolved without sequalae.

One subject experienced depressed mood after discharge from the Clinical Research Unit, which was considered not related to the study drug and was attributed to domestic circumstances.

One subject on active treatment and two subjects on placebo experienced hypervigilance (described as feeling an increase of alertness and/or more energy).

TABLE 5
Summary of Treatment-Emergent Adverse
Events (Safety Population) Part 2 MAD

| | | Compound A | | | | |
|---|---|---|---|---|---|---|
| | Placebo | 3 mg | 8 mg | 15 mg | 25 mg | Total |
| | (N = 8) | (N = 6) | (N = 6) | (N = 6) | (N = 6) | (N = 24) |
| Number of Subjects | n (%) | n (%) | n (%) | n (%) | n (%) | n (%) |
| Any TEAE | 2 | 2 | 5 | 3 | 5 | 15 |
| | (25.0) | (33.3) | (83.3) | (50.0) | (83.3) | (62.5) |
| TEAE by Highest | | | | | | |
| Severity^{a} | | | | | | |
| Mild | 2 | 2 | 4 | 3 | 4 | 13 |
| | (25.0) | (33.3) | (66.7) | (50.0) | (66.7) | (54.2) |
| Moderate | 0 | 0 | 1 | 0 | 1 | 2 |
| | | | (16.7) | | (16.7) | (8.3) |
| Severe | 0 | 0 | 0 | 0 | 0 | 0 |
| TEAE by Highest | | | | | | |
| Relationship^{a} | | | | | | |
| Not Related | 0 | 2 | 3 | 0 | 0 | 5 |
| | | (33.3) | (50.0) | | | (20.8) |
| Related | 2 | 0 | 2 | 3 | 5 | 10 |
| | (25.0) | | (33.3) | (50.0) | (83.3) | (41.7) |
| AE Leading to Study | 0 | 0 | 0 | 0 | 1 | 1 |
| Drug Discontinuation | | | | | (16.7) | (4.2) |
| Any SAEs | 0 | 0 | 0 | 0 | 0 | 0 |
| Death | 0 | 0 | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.
^{a}If a subject has multiple AEs, the highest severity is presented in summary by severity, and the closest relationship to study drug is presented in summary by relationship.

TABLE 6
Summary of Treatment Emergent Adverse Events by
Preferred Term (Safety Population) Part 2 MAD

| | | Compound A | | | | |
|---|---|---|---|---|---|---|
| | Placebo | 3 mg | 8 mg | 15 mg | 25 mg | Total |
| | (N = 8) | (N = 6) | (N = 6) | (N = 6) | (N = 6) | (N = 24) |
| Preferred Term | n (%) | n (%) | n (%) | n (%) | n (%) | n (%) |
| Subjects with at | 2 | 2 | 5 | 3 | 5 | 15 |
| least one TEAE | (25.0) | (33.3) | (83.3) | (50.0) | (83.3) | (62.5) |
| Dizziness | 0 | 0 | 0 | 3 | 3 | 6 |
| | | | | (50.0) | (50.0) | (25.0) |
| Insomnia | 0 | 0 | 2 | 1 | 3 | 6 |
| | | | (33.3) | (16.7) | (50.0) | (25.0) |
| Pollakiuria | 0 | 0 | 0 | 1 | 2 | 3 |
| | | | | (16.7) | (33.3) | (12.5) |
| Visual impairment | 0 | 0 | 0 | 2 | 1 | 3 |
| | | | | (33.3) | (16.7) | (12.5) |
| Diarrhoea | 0 | 0 | 2 | 0 | 0 | 2 |
| | | | (33.3) | | | (8.3) |
| Abdominal pain | 0 | 0 | 1 | 0 | 0 | 1 |
| | | | (16.7) | | | (4.2) |
| Abdominal | 0 | 0 | 1 | 0 | 0 | 1 |
| pain lower | | | (16.7) | | | (4.2) |
| Abnormal dreams | 0 | 1 | 0 | 0 | 0 | 1 |
| | | (16.7) | | | | (4.2) |
| Back pain | 0 | 0 | 0 | 0 | 1 | 1 |
| | | | | | (16.7) | (4.2) |
| Balance disorder | 0 | 0 | 0 | 0 | 1 | 1 |
| | | | | | (16.7) | (4.2) |
| Blood pressure | 0 | 0 | 0 | 0 | 1 | 1 |
| systolic increased | | | | | (16.7) | (4.2) |
| Cough | 0 | 1 | 0 | 0 | 0 | 1 |
| | | (16.7) | | | | (4.2) |
| Depressed mood | 0 | 0 | 1 | 0 | 0 | 1 |
| | | | (16.7) | | | (4.2) |
| Euphoric mood | 0 | 0 | 0 | 0 | 1 | 1 |
| | | | | | (16.7) | (4.2) |
| Feeling of | 0 | 0 | 0 | 0 | 1 | 1 |
| relaxation | | | | | (16.7) | (4.2) |
| Headache | 0 | 0 | 0 | 1 | 0 | 1 |
| | | | | (16.7) | | (4.2) |
| Hypervigilance | 2 | 0 | 1 | 0 | 0 | 1 |
| | (25.0) | | (16.7) | | | (4.2) |
| Nocturia | 0 | 0 | 1 | 0 | 0 | 1 |
| | | | (16.7) | | | (4.2) |
| Photophobia | 0 | 0 | 1 | 0 | 0 | 1 |
| | | | (16.7) | | | (4.2) |
| Salivary | 0 | 0 | 0 | 0 | 1 | 1 |
| hypersecretion | | | | | (16.7) | (4.2) |
| Vomiting | 0 | 0 | 1 | 0 | 0 | 1 |
| | | | (16.7) | | | (4.2) |

Abbreviations: TEAE = treatment emergent adverse event, N = number of subjects dosed with each drug (on Day 1), n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.

### Pharmacokinetics

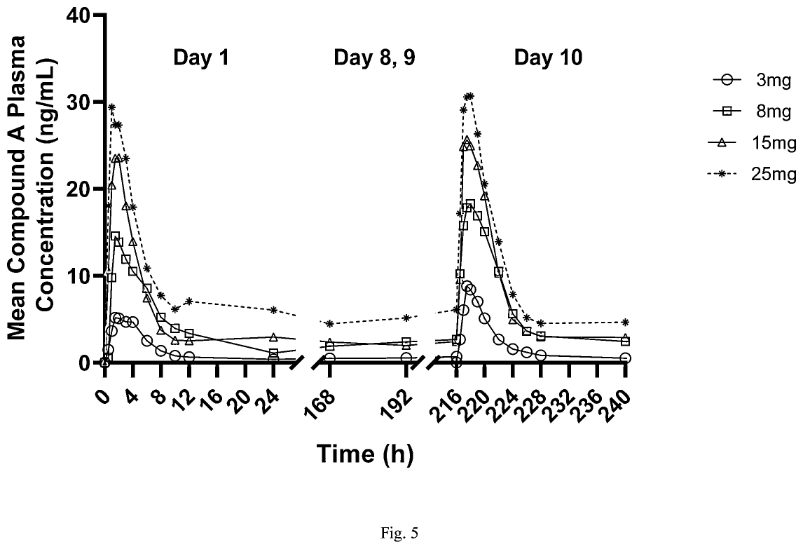

Compound A was generally quantifiable up to 48 hours post-dose across both doses. Post-dose concentrations were characterized by a multi-exponential decline. Following administration of multiple oral dose (3, 8, 15 and 25 mg) for 10 days, Compound A showed a moderate absorption rate with a median Tₘₐₓ of 1.75 hours with a range 1.0 to 6.0 hours for both Day 1 and Day 10. The PK profile was biphasic with a terminal t_{1/2} of 8 to 16 hours on Day 10 following multiple oral dose administration of Compound A (FIG. 5).

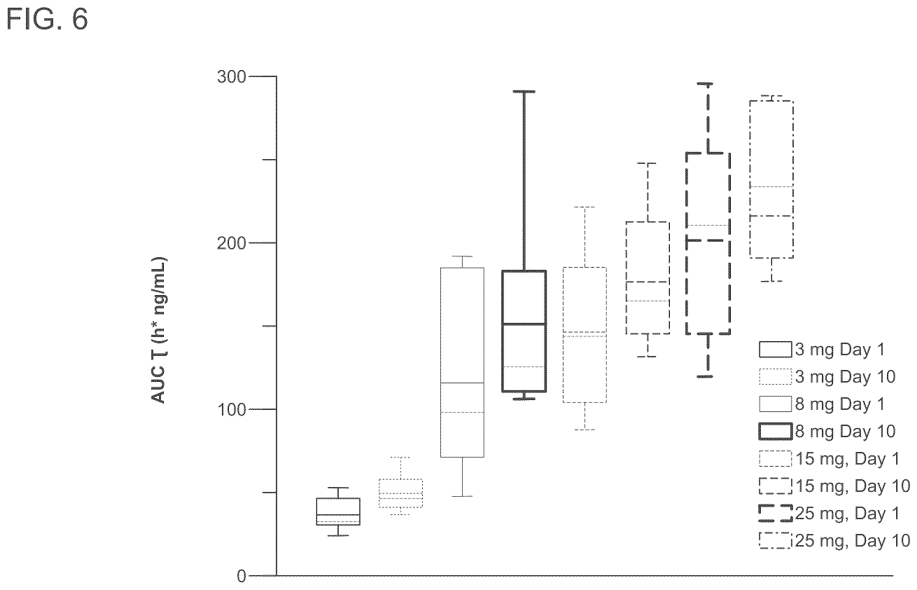

Across the doses studied, geometric mean Cₘₐₓ increased less than dose proportionally while geometric mean AUC (tau) increased dose proportionally with increasing doses suggesting increase in bioavailability with increasing dose from 3 to 25 mg (FIG. 6). Subsequently, the accumulation ratio (Day 10/Day 1) Cₘₐₓ for Compound A was 1.2 to 1.7 and accumulation ratio (Day 10/Day 1) AUC₀₋₂₄ was 1 to 1.4 over the range of 3 to 25 mg doses (Table 7).

Accumulation can be attributed to the drug being cleared slowly compared to its absorption rate. With the half-life of 8 to 10 hours the steady state is expected to be achieved by Day 4.

TABLE 7
Summary of Pharmacokinetic Parameters of Compound A Following
Multiple Oral Doses of Compound A - Part 2 (MAD)

| | Compound A Dose and Timepoint | | | | | | | |
|---|---|---|---|---|---|---|---|---|
| PK | 3 mg | | 8 mg | | 15 mg | | 25 mg | |
| Parameters | N = 6 | | N = 6 | | N = 6 | | N = 6 | |
| (unit) | Day 1 | Day 10 | Day 1 | Day 10 | Day 1 | Day 10 | Day 1 | Day 10 |
| Cₘₐₓ | 5.60 | 9.38 | 16.0 | 22.1 | 25.2 | 28.9 | 28.0 | 36.9 |
| (ng/mL) | (17.7) | (25.2) | (26.7) | (19.4) | (35.8) | (35.5) | (78.9) | (27.5) |
| C₈ | 1.21 | 1.39 | 3.89 | 4.6 | 3.41 | 4.62 | 7.35 | 7.39 |
| (ng/mL) | (58.0) | (56.8) | (93.1) | (73.6) | (52.5) | (42.9) | (35.0) | (41.9) |
| Tₘₐₓ | 1.75 | 1.5 | 1.75 | 1.75 | 1.5 | 1.75 | 1.25 | 1.5 |
| (h) | (1.0-4.0) | (1.0-3.0) | (1.0-6.0) | (1.5-4.0) | (1.0-4.0) | (1.0-4.0) | (1.0-3.0) | (1.0-3.0) |
| t_{1/2} | NA | 9.63 | NA | 7.66 | NA | 14.0 | NA | 15.9 |
| (h) | | (65.3) | | (68.3)^{a} | | (46.6)^{a} | | (87.1)^{c} |
| C_{trough} | NA | 0.495 | NA | 2.07 | NA | 2.06 | NA | 4.51 |
| (ng/mL) | | (60.6) | | (54.0) | | (49.8) | | (42.2) |
| AUC₀₋₂₄, ss | 35.5 | 48.4 | 104 | 141 | | | | |
| (ng · h/mL) | (30.0) | (25.8) | (57.5) | (39.0) | | | | |
| CL/F | NA | 62.0 | NA | 55.7 | NA | 84.4 | NA | 108 |
| (L/h) | | (25.8) | | (43.8)^{a} | | (25.8)^{a} | | (19.4)^{c} |
| Vz/F | NA | 862 | NA | 616 | NA | 1705 | NA | 2481 |
| (L) | | (87.7) | | (104)^{a} | | (29.0)^{a} | | (80.4)c |

| | | | | |
|---|---|---|---|---|
| Accumulation | 1.68 | 1.38 | 1.15 | 1.32 |
| ratio | | | | |
| (Day 10/Day 1) | | | | |
| Cₘₐₓ | | | | |
| (ng · h/mL) | | | | |
| Accumulation | 1.36 | 1.36 | 1.23 | 1.02 |
| ratio | | | | |
| (Day 10/Day 1) | | | | |
| AUC₀₋₂₄ | | | | |
| (ng · h/mL) | | | | |

Abbreviations: AUC = area under the concentration-time curve, C₈ = Concentration at 8-hour postdose, Cₘₐₓ = maximum plasma concentration, C_{trough} = trough concentration, MAD = multiple ascending dose, NA = not applicable, PK = pharmacokinetics, t_{1/2} = half-life, Tₘₐₓ = time to reach Cₘₐₓ, Vz/F = apparent volume of distribution at terminal phase, CV % = coefficient of variation percentage.
^{a}N = 5 the rest of subjects had unaccepted R2 or extrapolated AUC > 30%.
Data represented as geometric mean (geometric CV %). Tₘₐₓ is represented as median (range).

4.18.3 Part 3 (RelBA with FE)

The study included three administrations of 10 mg Compound A consisting of 1 dose of Compound A powder-in-capsule formulation, and 2 doses of Compound A 10 mg tablet formulation (under fasting and fed conditions). A total of 12 subjects were enrolled in Part 3 (RelBA with FE), receiving all three specified doses of Compound A. All subjects were healthy adult subjects with no clinically significant illness or abnormality.

### Safety

Treatment with Compound A was well tolerated in healthy subjects under fed and fasted conditions with a similar AE profile to that observed in Parts 1 and 2. No deaths, serious AEs, or AEs leading to discontinuation occurred during this part of the study. Most of the subjects (11 of 12) reported at least one AE. Except for a Catheter site phlebitis (phlebitis of the old intravenous cannula site) reported as moderate in severity, all TEAEs in Part 3 were mild and did not require medical intervention. TEAEs that occurred in more than 1 subject were headache, dizziness, hypervigilance (described as feeling more alertness or more energy), insomnia, and pollakiuria. Most of these events were considered related to the study drug. There were no clinically significant abnormalities or notable trends observed following study drug administration, in the safety variables of chemistry or hematology laboratory findings, vital signs, or ECG measurements. Additionally, there was no apparent impact of food (neither fed nor fasted), or formulation on safety parameters.

### Pharmacokinetics

### 4.18.3.1 Tablet vs Powder-in-Capsule

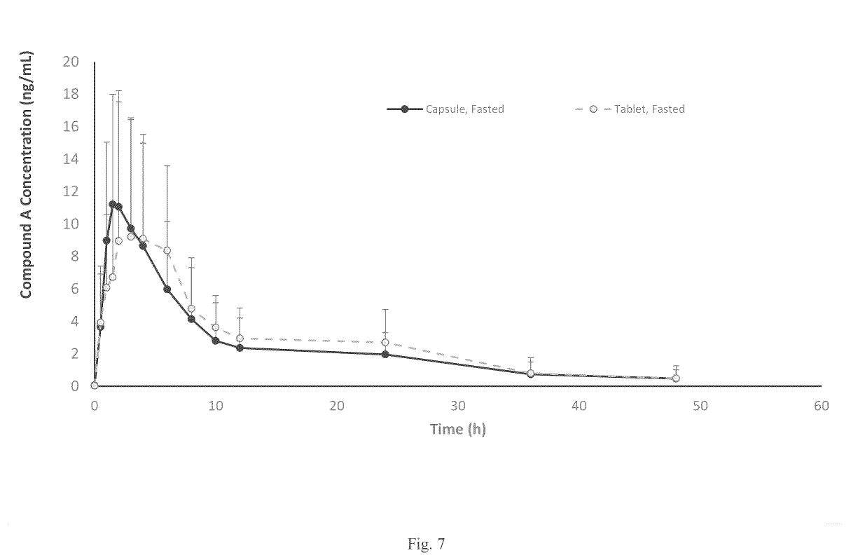

Overall mean concentration-time profile for Compound A after administration of tablet and powder-in-capsule formulation were comparable (FIG. 7). The relative exposure of 10 mg tablet formulation was slightly higher as compared to that obtained after 10 mg powder-in-capsule formulation. The geometric mean Compound A Cₘₐₓ values were 10.8 ng/ml and 11.8 ng/ml for tablet and powder-in-capsule, respectively and Cₘₐₓ ratio (tablet Cₘₐₓ/capsule Cₘₐₓ) was 0.9. The geometric mean Compound A AUC₀% values were 144 ng*h/mL and 124 ng*h/mL for tablet and powder-in-capsule, respectively. AUC_{0-∞} ratio was 1.2.

### 4.18.3.2 Food Effect (Cohort 1 and 2)

Food effect was studied following a high fat (cohort 1) and low fat (cohort 2) meal. Both cohorts were completed in independent studies. For cohort 1, a high-fat meal was served consisting of 800-1,000 total calories with 150 calories from protein, 250 calories from carbohydrates, 500-600 calories from fat (≥50 percent of calories from fat). For cohort 2, a low-fat meal was served consisting of 400-500 total calories with 100-125 calories from fat (25 percent of calories from fat).

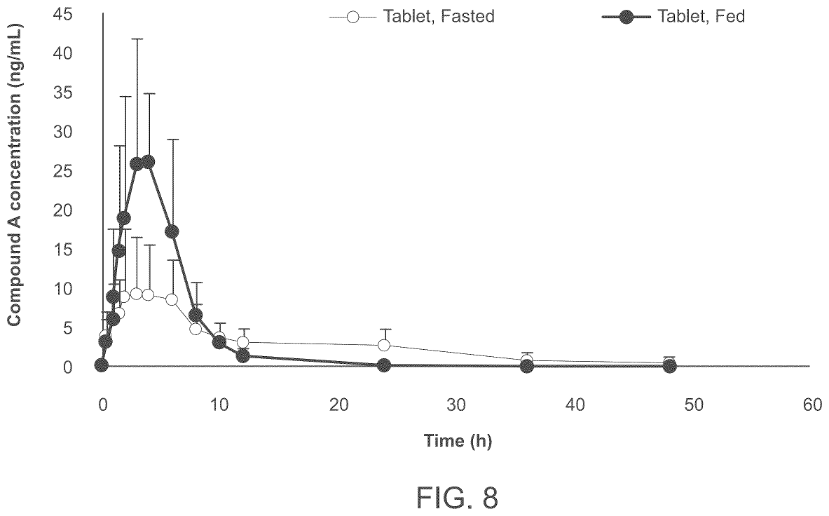

For cohort 1, mean concentration-time profile for Compound A after administration of tablet under fasting and fed (high-fat) condition is presented in FIG. 8. Administration of Compound A tablet in fed condition increased peak plasma concentration by 3-fold [Cₘₐₓ: 10.8 ng/ml in fasted vs. 30.1 ng/mL in fed condition] while no impact on extent of absorption. Food did not impact time to peak concentration; median Tₘₐₓ was 3.5 hours (range: 1.0-6.0) for both fed and fasted condition (Table 8). There was a decrease in half-life to 2.8 hours.

TABLE 8
Summary of Plasma PK Parameters for Compound
A Following Single Oral Doses of Compound
A - Part 3 (RelBA with FE) (Cohort 1)

| | Tablet (Fasted) | | Capsule (Fasted) | | Tablet (Fed) | |
|---|---|---|---|---|---|---|
| PK Parameters | N = 12 | | N = 12 | | N = 12 | |
| Cₘₐₓ (ng/mL) | 10.8 | [62.9] | 11.8 | [54.0] | 30.1 | [42.3] |
| Tₘₐₓ (h) | 3.5 | (1.0-6.0) | 2.0 | (1.0-8.0) | 3.5 | (1.0-6.0) |
| AUC_{0-∞} | 144 | [66.2.] ^{a} | 124 | [67.7] ^{b} | 140 | [50.6] |
| (ng*h/mL) | | | | | | |
| t_{1/2} (h) | 11.0 | [41.7] ^{a} | 10.8 | [43.2.] ^{b} | 2.8 | [52.3] |

^{a} N = 8, the rest of subjects had unaccepted R2 or extrapolated AUC > 30%.
^{b} N = 9, the rest of subjects had unaccepted R2 or extrapolated AUC > 30%.

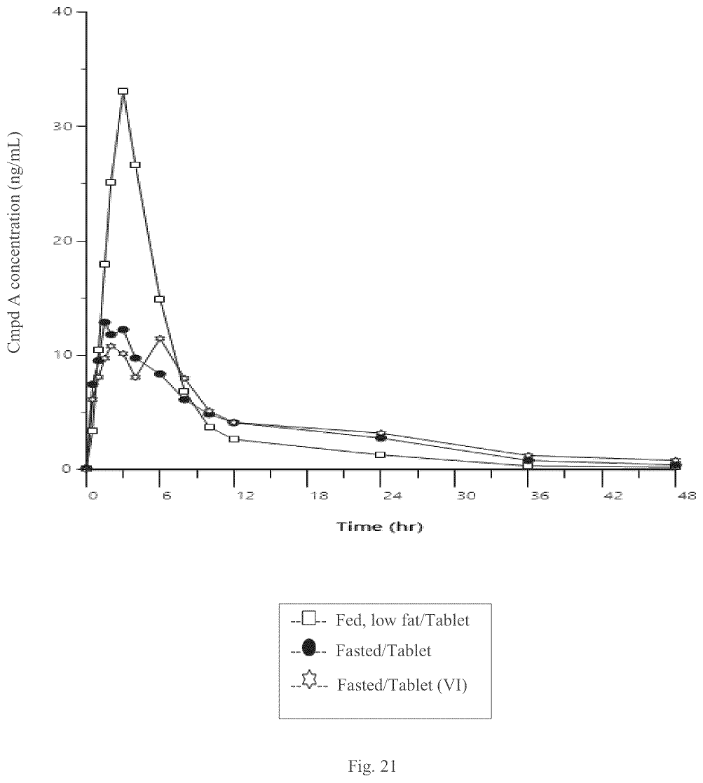

For cohort 2, mean concentration-time profile for Compound A after administration of tablet under fasting and fed (low-fat) condition is presented in FIG. 21. Administration of Compound A tablet in fed conditions increased peak plasma concentration by approximately 2-fold [Cₘₐₓ: 14.2 ng/ml in fasted vs. 32.1 ng/mL in fed condition] when compared to fasted conditions. The extent of absorption in low-fat fed conditions was slightly higher than in fasted conditions. In Cohort 2, median Tₘₐₓ was higher in low-fat fed conditions than in fasted conditions, while low-fat food did not have a major impact in t_{1/2} (Table 8A).

TABLE 8A
Summary of Plasma PK Parameters for Compound
A Following Single Oral Doses of Compound
A - Part 3 (RelBA with FE) (Cohort 2)

| | Tablet (Fasted) | Tablet (Fed) |
|---|---|---|
| PK Parameters | N = 12 | N = 12 |
| Cₘₐₓ (ng/mL) | 14.2 | 32.1 |
| Tₘₐₓ (h) | 2.5 | 3.01 |
| AUC_{0-∞} | 189.1 ^{a} | 179.5 (33.0) ^{b} |
| (ng*h/mL) | | |
| t_{1/2} (h) | 9.22 ^{a} | 9.69 ^{b} |

^{a} N = 8.
^{b} N = 10.

### 4.18.4 Part 4 (NT1 Patients)

Four subjects with NT1 were dosed in Part 4 (POC in NT1) in a randomized 4-sequence 4-period cross-over design. Each subject received three doses of Compound A, including 1, 3, and 8 mg, and a single dose of placebo. Each dose of Compound A or matching placebo was separated by 2 days. A 14-day washout period preceded the treatment period and included discontinuation of narcolepsy and cataplexy medications. All subjects underwent the washout period and completed all 4-way randomized crossover periods.

All subjects were white, not hispanic or latino, and the majority (3 out of 4) were males. Subjects age ranged from 19 to 33 years, with a mean age of approximately 24 years. Subject BMI ranged from 25.1 to 38.0 kg/m², with a mean BMI of approximately 31 kg/m². Significant severity of narcolepsy symptoms at baseline, following the washout period, was observed as measured by WCR, NSS, and ESS (Table 9).

TABLE 9
Severity of Narcolepsy Symptoms at Baseline - Part 4 POC NT1

| Assessment | n | Mean (SD) | | Median | Min, Max |
|---|---|---|---|---|---|
| WCR | 4 | 9.0 | (10.61) | 5.5 | 1, 24 |
| NSS | 4 | 39.8 | (3.50) | 39.5 | 36, 44 |
| ESS | 4 | 16.0 | (2.83) | 17 | 12, 18 |

Abbreviations: ESS = Epworth Sleepiness Scale, Min = minimum, Max = maximum, NSS = Narcolepsy Severity Scale, SD = standard deviation, WCR = weekly cataplexy rate.
Baseline is defined as the last non-missing value on or before the first dose of study drug.
Percentages are based on the number of subjects in the Safety Population in each column (N).
NSS severity ranks: mild 0-14, moderate 15-28, severe 29-42, very severe 43-57 (Dauvillers 2020).
Excessive daytime sleepiness ranges on ESS: mild 11-12, moderate 13-15, severe 16-24 (Johns 2023)

### Safety

Treatment with Compound A was well tolerated in all doses of Part 4 (NT1). No deaths, serious AEs, or AEs leading to discontinuation occurred during this part of the study (Table 10). All four subjects had at least one TEAE and all TEAEs were considered mild in severity (Table 10). The incidence of TEAEs was observed to generally increase with a higher dose (Table 11). All drug related TEAEs were observed on 8 mg dose. The most common (reported in more than 1 subject) drug related TEAEs reported were insomnia, pollakiuria, and salivary hypersecretion. No drug related clinically significant changes from baseline were observed in clinical chemistry, including hepatic and renal parameters, creatinine kinase, and electrolytes.

Of note, one NT1 subject experienced a non-treatment-emergent AE of steatohepatitis (reported as acute steatohepatitis), which was mild in severity. This subject had elevated liver function tests (LFT) prior to dose administration, including elevation of ALT and AST on Day −2 (174 U/L and 88 U/L, respectively), which was considered to be due to the shifted dietary patterns during the screening and washout periods: the subject consumed products rich in fat and carbohydrates content, aiming to overcome excessive daytime sleepiness. The ALT and AST values reached a maximum on Day 4 and were 234 U/L and 129 U/L, respectively, and reduced gradually after. The AE of liver steatosis was considered resolved on Day 14 of the study. The serum bilirubin was normal throughout. Additional AEs in this subject included weight increased (reportedly 8.5 kg during screening), hyperlipidemia, application site dermatitis (due to ECG leads), and pollakiuria.

TABLE 10
Summary of Treatment Emergent Adverse Events (Safety Population) Part 4 POC NT1

| | | Compound A | | | |
|---|---|---|---|---|---|
| | | Compound A | Compound A | Compound A | |
| | Placebo | 1 mg | 3 mg | 8 mg | Total |
| | (N = 4) | (N = 4) | (N = 4) | (N = 4) | (N = 4) |
| Number of Subjects | n (%) | n (%) | n (%) | n (%) | n (%) |
| Any TEAE | 2 (50.0) | 1 (25.0) | 2 (50.0) | 4 (100) | 4 (100) |
| TEAE by Highest | | | | | |
| Severity^{a} | | | | | |
| Mild | 2 (50.0) | 1 (25.0) | 2 (50.0) | 4 (100) | 4 (100) |
| Moderate | 0 | 0 | 0 | 0 | 0 |
| Severe | 0 | 0 | 0 | 0 | 0 |
| TEAE by Highest | | | | | |
| Relationship^{a} | | | | | |
| Not Related | 2 (50.0) | 1 (25.0) | 2 (50.0) | 0 | 0 |
| Related | 0 | 0 | 0 | 4 (100) | 4 (100) |
| AE Leading to Study | 0 | 0 | 0 | 0 | 0 |
| Drug Discontinuation | | | | | |
| Any SAEs | 0 | 0 | 0 | 0 | 0 |
| Death | 0 | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group.
The percentages are based on the total number of subjects in the safety population in each column (N).
Adverse events are coded using MedDRA version 26.0.
TEAEs are defined as AEs that occur or worsen on or after the first dose of study drug.
If a subject experiences more than one adverse event in a category, the subject is counted only once in that category.
^{a}If a subject has multiple AEs, the highest severity is presented in summary by severity, and the closest relationship to study drug is presented in summary by relationship.

TABLE 11
Summary of Treatment Emergent Adverse Events by
Preferred Term (Safety Population) Part 4 POC NT1

| | | Compound A | | | | | |
|---|---|---|---|---|---|---|---|
| | | Compound A | Compound A | Compound A | | | |
| | Placebo | 1 mg | 3 mg | 8 mg | | Total | |
| | (N = 4) | (N = 4) | (N = 4) | (N = 4) | | (N = 4) | |
| Preferred Term | n (%) | n (%) | n (%) | n (%) | | n (%) | |
| Subjects with at | 2 (50.0) | 1 (25.0) | 2 (50.0) | 4 | (100) | 4 | (100) |
| least one TEAE | | | | | | | |
| Insomnia | 0 | 0 | 0 | 3 | (75.0) | 3 | (75.0) |
| Application | 0 | 1 (25.0) | 0 | 1 | (25.0) | 2 | (50.0) |
| site dermatitis | | | | | | | |
| Pollakiuria | 0 | 0 | 0 | 2 | (50.0) | 2 | (50.0) |
| Salivary | 0 | 0 | 0 | 2 | (50.0) | 2 | (50.0) |
| hypersecretion | | | | | | | |
| Blood pressure | 0 | 0 | 0 | 1 | (25.0) | 1 | (25.0) |
| increased | | | | | | | |
| Bruxism | 0 | 0 | 0 | 1 | (25.0) | 1 | (25.0) |
| Dermatitis contact | 0 | 0 | 1 (25.0) | 0 | | 1 | (25.0) |
| Dizziness | 0 | 0 | 0 | 1 | (25.0) | 1 | (25.0) |
| Hyperhidrosis | 0 | 0 | 0 | 1 | (25.0) | 1 | (25.0) |

| | | | | | | |
|---|---|---|---|---|---|---|
| Procedural anxiety | 0 | 0 | 1 (25.0) | 0 | 1 | (25.0) |
| Vascular access | 0 | 0 | 1 (25.0) | 0 | 1 | (25.0) |
| site injury | | | | | | |
| Vessel puncture | 0 | 0 | 1 (25.0) | 0 | 1 | (25.0) |
| site bruise | | | | | | |

| | | | | | |
|---|---|---|---|---|---|
| Anaemia | 1 (25.0) | 0 | 0 | 0 | 0 |
| Hyperlipidaemia | 1 (25.0) | 0 | 0 | 0 | 0 |

Abbreviations: TEAE = treatment emergent adverse event, N = number of subjects dosed with each drug (on Day 1), n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.

### Pharmacokinetics

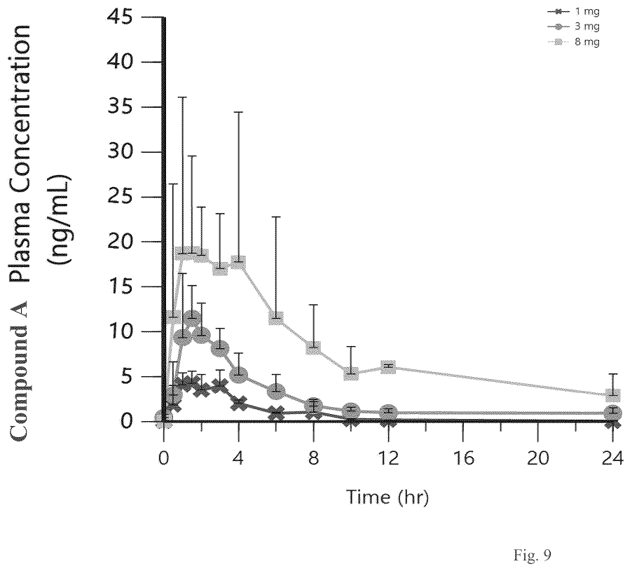

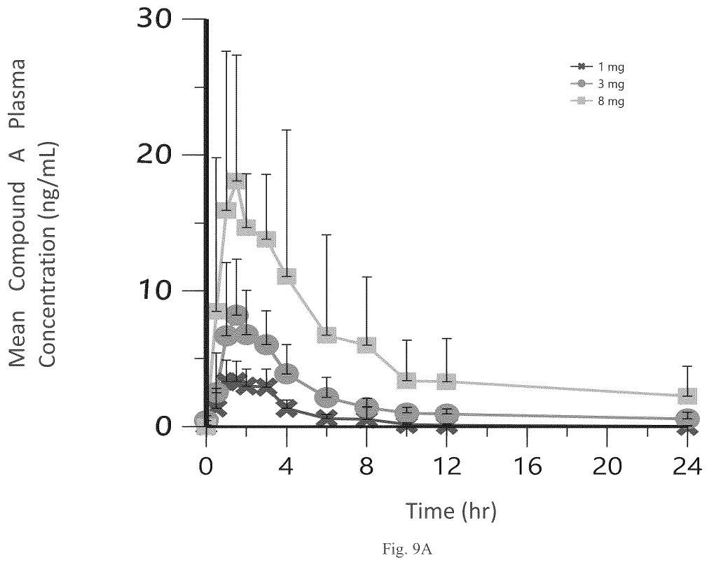

Mean concentration-time profile for Compound A in patients with NT1 is presented in FIG. 9 and key PK parameters are presented in Table 12. Following administration of single dose (1, 3 and 8 mg), Compound A showed a moderate absorption rate with a median Tₘₐₓ of 1.5 with a range 0.5 to 4.0 hours. Systemic exposure to Compound A increased dose proportionally.

TABLE 12
Summary of Plasma PK Parameters for Compound A Following
Single Oral Doses of Compound A - Part 4 (NT1)

| | 1 mg | | 3 mg | | 8 mg | |
|---|---|---|---|---|---|---|
| PK Parameters | N = 3^{a} | | N = 4 | | N = 3^{a} | |
| Cₘₐₓ (ng/mL) | 4.395 | (30.3) | 11.607 | (33.8) | 27.859 | (57.1) |
| Tₘₐₓ (h) | 1.5 | (0.5, 3.0) | 1.5 | (1.0, 2.0) | 3.0 | (1.0, 4.0) |
| AUC₀₋₂₄ | 19.493 | (21.6) | 59.134 | (16.6) | 168.509 | (57.3) |
| (ng*h/mL) | | | | | | |

Abbreviations: AUC₀₋₂₄ = area under the concentration-time curve from 0 to 24 hours, Cₘₐₓ = maximum plasma concentration, Tₘₐₓ = time to reach Cₘₐₓ, CV % = coefficient of variation percentage.
^{a}One subject had limited PK samples after receiving 1 mg and 8 mg doses; these timepoints were excluded from NCA analysis. This subject had sufficient timepoints after 3 mg dosing.
Data represented as geometric mean (geometric CV %). Tmax is represented as median (range).

### 4.18.4a Part 4 (NT1 Patients)

Ten subjects with NT1 (including the four patients described above in 4.18.4) were dosed in Part 4 (POC in NT1) in a randomized 4-sequence 4-period cross-over design. With one exception, each subject received three doses of Compound A, including 1, 3, and 8 mg, and a single dose of placebo. One patient withdrew from the study after the first dose due to venous access complications. Each dose of Compound A or matching placebo was separated by 2 days. A 14-day washout period preceded the treatment period and included discontinuation of narcolepsy and cataplexy medications. With the exception of one patient as noted above, all subjects underwent the washout period and completed all 4-way randomized crossover periods.

All subjects were white, not hispanic or latino, and the majority (6 out of 10) were females. Subjects age ranged from 19 to 53 years, with a mean age of approximately 25.6 years. Subject BMI ranged from 22.0 to 38.0 kg/m², with a mean BMI of approximately 26.5 kg/m². Significant severity of narcolepsy symptoms at baseline, following the washout period, was observed as measured by WCR, NSS, and ESS (Table 9a).

TABLE 9a
Severity of Narcolepsy Symptoms at Baseline - Part 4 POC NT1

| Assessment | n | Mean (SD) | | Min, Max |
|---|---|---|---|---|
| WCR | 10 | 32.0 | (43.79) | 0, 145 |
| NSS | 10 | 40.6 | (7.26) | 28, 54 |
| ESS | 10 | 15.9 | (2.47) | 12, 19 |

Abbreviations: ESS = Epworth Sleepiness Scale, Min = minimum, Max = maximum, NSS = Narcolepsy Severity Scale, SD = standard deviation, WCR = weekly cataplexy rate.
Baseline is defined as the last non-missing value on or before the first dose of study drug.
Percentages are based on the number of subjects in the Safety Population in each column (N).
NSS severity ranks: mild 0-14, moderate 15-28, severe 29-42, very severe 43-57 (Dauvillers 2020).
Excessive daytime sleepiness ranges on ESS: mild 11-12, moderate 13-15, severe 16-24 (Johns 2023)

### Safety

Treatment with Compound A was well tolerated in all doses of Part 4 (NT1). No deaths, serious AEs, or TEAEs leading to discontinuation occurred during this part of the study (Table 10a). Nine subjects had at least one TEAE and most TEAEs were considered mild in severity (Table 10a). One TEAE (nausea) was considered moderate in severity (at the highest dose). The incidence of TEAEs was observed to generally increase with a higher dose (Table 11a). The majority of drug related TEAEs were observed on 8 mg dose. The most common (reported in more than 1 subject) drug related TEAEs reported were pollakiuria, insomnia, middle insomnia, and salivary hypersecretion. No drug related clinically significant changes from baseline were observed in clinical chemistry, including hepatic and renal parameters, creatinine kinase, and electrolytes.

Of note, one NT1 subject experienced a non-treatment-emergent AE of steatohepatitis (reported as acute steatohepatitis), which was mild in severity. This subject had elevated liver function tests (LFT) prior to dose administration, including elevation of ALT and AST on Day −2 (174 U/L and 88 U/L, respectively), which was considered to be due to the shifted dietary patterns during the screening and washout periods: the subject consumed products rich in fat and carbohydrates content, aiming to overcome excessive daytime sleepiness. The ALT and AST values reached a maximum on Day 4 and were 234 U/L and 129 U/L, respectively, and reduced gradually after. The AE of liver steatosis was considered resolved on Day 14 of the study. The serum bilirubin was normal throughout. Additional AEs in this subject included weight increased (reportedly 8.5 kg during screening), hyperlipidemia, application site dermatitis (due to ECG leads), and pollakiuria.

TABLE 10a
Summary of Treatment Emergent Adverse Events
(Safety Population) Part 4 POC NT1

| | | Compound A | | |
|---|---|---|---|---|
| | | Compound A | Compound A | Compound A |
| | Placebo | 1 mg | 3 mg | 8 mg |
| | (N = 9) | (N = 9) | (N = 9) | (N = 10) |
| Number of Subjects | n (%) | n (%) | n (%) | n (%) |
| Any TEAE | 4 (44.4) | 6 (66.7) | 5 (55.6) | 9 (90.0) |
| TEAE by Highest | | | | |
| Severity^{a} | | | | |
| Mild | 4 (44.4) | 6 (66.7) | 5 (55.6) | 8 (80.0) |
| Moderate | 0 | 0 | 0 | 1 (10.0) |
| Severe | 0 | 0 | 0 | 0 |
| TEAE by Highest | | | | |
| Relationship^{a} | | | | |
| Not Related | 3 (33.3) | 1 (11.1) | 2 (22.2) | 0 |
| Related | 1 (11.1) | 5 (55.6) | 3 (33.3) | 9 (90.0) |
| AE Leading to Study | 0 | 0 | 0 | 0 |
| Drug Discontinuation | | | | |
| Any SAEs | 0 | 0 | 0 | 0 |
| Death | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group.
The percentages are based on the total number of subjects in the safety population in each column (N).
Adverse events are coded using MedDRA version 26.0.
TEAEs are defined as AEs that occur or worsen on or after the first dose of study drug.
If a subject experiences more than one adverse event in a category, the subject is counted only once in that category.
^{a}If a subject has multiple AEs, the highest severity is presented in summary by severity, and the closest relationship to study drug is presented in summary by relationship.

TABLE 11a
Summary of Treatment Emergent Adverse Events by
Preferred Term (Safety Population) Part 4 POC NT1

| | | Compound A | | | |
|---|---|---|---|---|---|
| | | Compound A | Compound A | Compound A | |
| | Placebo | 1 mg | 3 mg | 8 mg | Total |
| | (N = 9) | (N = 9) | (N = 9) | (N = 10) | (N = 10) |
| Preferred Term | n (%) | n (%) | n (%) | n (%) | n (%) |
| Subjects with at | 4 (44.4) | 6 (66.7) | 5 (55.6) | 9 (90.0) | 9 (90.0) |
| least one TEAE | | | | | |
| Pollakiuria | 0 | 0 | 2 (22.2) | 4 (40.0) | 4 (40.0) |
| Insomnia | 0 | 0 | 0 | 3 (30.0) | 3 (30.0) |
| Middle Insomnia | 0 | 0 | 1 (11.1) | 3 (30.0) | 3 (30.0) |
| Salivary | 1 (11.1) | 1 (11.1) | 1 (11.1) | 3 (30.0) | 3 (30.0) |
| hypersecretion | | | | | |
| Vessel puncture | 0 | 0 | 1 (11.1) | 2 (20.0) | 3 (30.0) |
| site bruise | | | | | |
| Application | 0 | 1 (11.1) | 0 | 1 (10.0) | 2 (20.0) |
| site dermatitis | | | | | |
| Decreased | 0 | 1 (11.1) | 0 | 1 (10.0) | 2 (20.0) |
| appetite | | | | | |
| Dermatitis | 0 | 0 | 2 (22.2) | 0 | 2 (20.0) |
| contact | | | | | |
| Dizziness | 0 | 1 (11.1) | 0 | 2 (20.0) | 2 (20.0) |
| Nausea | 1 (11.1) | 2 (22.2) | 0 | 2 (20.0) | 2 (20.0) |
| Vessel puncture | 0 | 0 | 0 | 2 (20.0) | 2 (20.0) |
| site pain | | | | | |
| Back pain | 0 | 0 | 0 | 1 (10.0) | 1 (10.0) |
| Blood pressure | 0 | 0 | 0 | 1 (10.0) | 1 (10.0) |
| increased | | | | | |
| Bruxism | 0 | 0 | 0 | 1 (10.0) | 1 (10.0) |
| Dehydration | 0 | 1 (11.1) | 0 | 0 | 1 (10.0) |
| Haemoglobin | 0 | 0 | 0 | 1 (10.0) | 1 (10.0) |
| decreased | | | | | |
| Hyperhidrosis | 0 | 0 | 0 | 1 (10.0) | 1 (10.0) |
| Sinus tachycardia | 0 | 1 (11.1) | 0 | 1 (10.0) | 1 (10.0) |
| Vascular access | 0 | 0 | 1 (11.1) | 0 | 1 (10.0) |
| site injury | | | | | |
| Anaemia | 1 (11.1) | 0 | 0 | 0 | 0 |
| Headache | 1 (11.1) | 0 | 0 | 0 | 0 |
| Hyperlipdaemia | 1 (11.1) | 0 | 0 | 0 | 0 |

Abbreviations: TEAE = treatment emergent adverse event, N = number of subjects dosed with each drug (on Day 1), n = number of subjects with adverse events in a group.
Adverse events are coded using MedDRA version 26.0.
TEAEs are defined as AEs that occur or worsen on or after the first dose of study drug.
If a subject experiences more than one adverse event in a category, the subject is counted only once in that category.

### Pharmacokinetics

Mean concentration-time profile for Compound A in patients with NT1 is presented in FIG. 9A.

4.18.5 Part 5 (NT2 patients)

Nine subjects with NT2 were dosed in Part 5 (POC in NT2) in a randomized 4-sequence 4-period cross-over design. Each dose of Compound A or matching placebo was separated by 2 days. A 14-day washout period preceded the treatment period and included discontinuation of narcolepsy medications. All 9 subjects underwent the washout period and received at least one dose of Compound A. All subjects completed all 4-way randomized crossover periods. (These data correspond with the data from NT2 subjects reported in Examples 9 and 10.)

A majority of subjects were white (7 out of 9), all were non-Hispanic, and 5 out of 9 were females. Subjects ages ranged from 20 to 60 years, with a mean age of 36 years. Subjects' BMI ranged from 18.3 to 37.7 kg/m², with a mean BMI of approximately 26.0 kg/m². Moderate severity of narcolepsy symptoms and excessive daytime sleepiness was observed at baseline (measured on Day −2, after the 2-week washout period). Narcolepsy Severity Scale (NSS) score ranged from 12 to 32, with a mean NSS of 24.4. Epworth Sleepiness Scale (ESS) score ranged from 11 to 23, with a mean ESS of 15.9.

### Safety

Treatment with Compound A was well tolerated at all doses of Part 5 (NT2). Seven subjects (77.8%) had at least one TEAE. All but one TEAEs were considered mild in severity (Table 11b). One subject experienced pollakiuria which was moderate in severity and resolved in 3 days. TEAEs reported as related to the study drug were mostly observed on 25 mg dose. No AEs leading to discontinuation occurred during this part of the study. There were no serious adverse events (SAEs) or deaths.

The incidence of TEAEs by dose level is presented in Table 11c. TEAEs that were assessed as related to Compound A and observed in more than 1 subject included pollakiuria, dizziness, insomnia (including insomnia and initial insomnia). TEAEs that were assessed as related to Compound A and observed in only 1 subject included dizziness postural, feeling jittery, headache, photophobia, and urinary incontinence (Table 11d).

TABLE 11b
Summary of Treatment Emergent Adverse Events (Safety Population) Part 5 POC NT2

| | | Compound A | | | |
|---|---|---|---|---|---|
| | | Compound A | Compound A | Compound A | |
| | Placebo | 5 mg | 12 mg | 25 mg | Total |
| | (N = 9) | (N = 9) | (N = 9) | (N = 9) | (N = 9) |
| Number of Subjects | n (%) | n (%) | n (%) | n (%) | n (%) |
| Any TEAE | 2 (22.2) | 3 (33.3) | 4 (44.4) | 7 (77.8) | 7 (77.8) |
| TEAE by Highest | | | | | |
| Severity^{a} | | | | | |
| Mild | 2 (22.2) | 3 (33.3) | 4 (44.4) | 6 (66.7) | 6 (66.7) |
| Moderate | 0 | 0 | 0 | 1 (11.1) | 1 (11.1) |
| Severe | 0 | 0 | 0 | 0 | 0 |
| TEAE by Highest | | | | | |
| Relationship^{a} | | | | | |
| Not Related | 0 | 2 (22.2) | 3 (33.3) | 1 (11.1) | 1 (11.1) |
| Related | 2 (22.2) | 1 (11.1) | 1 (11.1) | 6 (66.7) | 6 (66.7) |
| AE Leading to Study | 0 | 0 | 0 | 0 | 0 |
| Drug Discontinuation | | | | | |
| Any SAEs | 0 | 0 | 0 | 0 | 0 |
| Death | 0 | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group, NT2 = narcolepsy type 2, POC = proof-of-concept.
Percentages are based on the total number of subjects in the safety population in each column (N).
Adverse events are coded using MedDRA version 26.0.
TEAEs are defined as AEs that occur or worsen on or after the first dose of study drug.
If a subject experiences more than one adverse event in a category, the subject is counted only once in that category.
^{a}If a subject has multiple AEs, the highest severity is presented in summary by severity, and the closest relationship to study drug is presented in summary by relationship.

TABLE 11c
Summary of Treatment Emergent Adverse Events by
Preferred Term (Safety Population) Part 5 POC NT2

| | | Compound A | | | |
|---|---|---|---|---|---|
| | | Compound A | Compound A | Compound A | |
| | Placebo | 5 mg | 12 mg | 25 mg | Total |
| | (N = 9) | (N = 9) | (N = 9) | (N = 9) | (N = 9) |
| Preferred Term | n (%) | n (%) | n (%) | n (%) | n (%) |
| Subjects with at | 2 (22.2) | 3 (33.3) | 4 (44.4) | 7 (77.8) | 7 (77.8) |
| least one TEAE | | | | | |
| Pollakiuria | 0 | 0 | 1 (11.1) | 3 (33.3) | 3 (33.3) |
| Dizziness | 0 | 0 | 0 | 2 (22.2) | 2 (22.2) |
| Insomnia | 1 (11.1) | 0 | 0 | 2 (22.2) | 2 (22.2) |
| Constipation | 0 | 0 | 1 (11.1) | 0 | 1 (11.1) |
| Contusion | 0 | 0 | 0 | 1 (11.1) | 1 (11.1) |
| Dizziness | 0 | 0 | 0 | 1 (11.1) | 1 (11.1) |
| postural | | | | | |
| Dry skin | 0 | 1 (11.1) | 0 | 0 | 1 (11.1) |
| Feeling jittery | 0 | 0 | 0 | 1 (11.1) | 1 (11.1) |
| Headache | 0 | 0 | 0 | 1 (11.1) | 1 (11.1) |
| Influenza | 0 | 0 | 1 (11.1) | 0 | 1 (11.1) |
| Initial insomnia | 0 | 1 (11.1) | 0 | 0 | 1 (11.1) |
| Nasal dryness | 0 | 0 | 0 | 1 (11.1) | 1 (11.1) |
| Nausea | 0 | 0 | 1 (11.1) | 0 | 1 (11.1) |
| Photophobia | 0 | 0 | 0 | 1 (11.1) | 1 (11.1) |
| Rash | 0 | 0 | 1 (11.1) | 0 | 1 (11.1) |
| Skin lesion | 0 | 1 (11.1) | 0 | 0 | 1 (11.1) |
| Urinary | 0 | 0 | 0 | 1 (11.1) | 1 (11.1) |
| incontinence | | | | | |
| Altered state of | 1 (11.1) | 0 | 0 | 0 | 0 |
| consciousness | | | | | |
| Tachycardia | 1 (11.1) | 0 | 0 | 0 | 0 |

Abbreviations: AE = adverse event, TEAE = treatment emergent adverse event, N = number of subjects dosed with each drug (on Day 1), n = number of subjects with adverse events in a group, NT2 = narcolepsy type 2, POC = proof-of-concept.
Adverse events are coded using MedDRA version 26.0.
TEAEs are defined as AEs that occur or worsen on or after the first dose of study drug.
If a subject experiences more than one adverse event in a category, the subject is counted only once in that category. If a subject experiences the same adverse event at multiple dose levels, the subject will be counted once per dose level and once in total.

TABLE 11d
Summary of Drug Related Treatment Emergent Adverse Events
by Preferred Term (Safety Population) Part 5 POC NT2

| | | Compound A | | | |
|---|---|---|---|---|---|
| | | Compound A | Compound A | Compound A | |
| | Placebo | 5 mg | 12 mg | 25 mg | Total |
| | (N = 9) | (N = 9) | (N = 9) | (N = 9) | (N = 9) |
| Preferred Term | n (%) | n (%) | n (%) | n (%) | n (%) |
| Subjects with at least one | 2 (22.2) | 1 (11.1) | 1 (11.1) | 6 (66.7) | 6 (66.7) |
| treatment-related TEAE | | | | | |
| Pollakiuria | 0 | 0 | 1 (1.1) | 3 (33.3) | 3 (33.3) |
| Dizziness | 0 | 0 | 0 | 2 (22.2) | 2 (22.2) |
| Insomnia | 1 (11.1) | 0 | 0 | 2 (22.2) | 2 (22.2) |
| Dizziness postural | 0 | 0 | 0 | 1 (11.1) | 1 (11.1) |
| Feeling jittery | 0 | 0 | 0 | 1 (11.1) | 1 (11.1) |
| Headache | 0 | 0 | 0 | 1 (11.1) | 1 (11.1) |
| Initial insomnia | 0 | 1 (11.1) | 0 | 0 | 1 (11.1) |
| Photophobia | 0 | 0 | 0 | 1 (11.1) | 1 (11.1) |
| Urinary incontinence | 0 | 0 | 0 | 1 (11.1) | 1 (11.1) |
| Altered state of | 1 (11.1) | 0 | 0 | 0 | 0 |
| consciousness | | | | | |
| Tachycardia | 1 (11.1) | 0 | 0 | 0 | 0 |

Abbreviations: AE = adverse event, TEAE = treatment emergent adverse event, N = number of subjects dosed with each drug (on Day 1), n = number of subjects with adverse events in a group, NT2 = narcolepsy type 2, POC = proof-of-concept.
Adverse events are coded using MedDRA version 26.0.
TEAEs are defined as AEs that occur or worsen on or after the first dose of study drug.
If a subject experiences more than one adverse event in a category, the subject is counted only once in that category. If a subject experiences the same adverse event at multiple dose levels, the subject will be counted once per dose level and once in total.

Single doses of Compound A were generally well tolerated at all doses tested. All TEAEs were mild in severity with the exception of 1 patient experiencing a moderate pollakiuria at 25 mg dose. No deaths, SAEs, or TEAEs leading to discontinuation. The majority of TEAEs assessed as related to Compound A were observed in the 25 mg dose. Drug-related TEAEs observed in more than 1 subject included pollakiuria, dizziness, insomnia (including insomnia and initial insomnia). No drug-related treatment-emergent clinically meaningful changes were identified in laboratory values, vital signs, or ECGs. Overall safety profile in NT2 subjects is generally consistent with NT1 and healthy volunteer populations.

### Pharmacokinetics

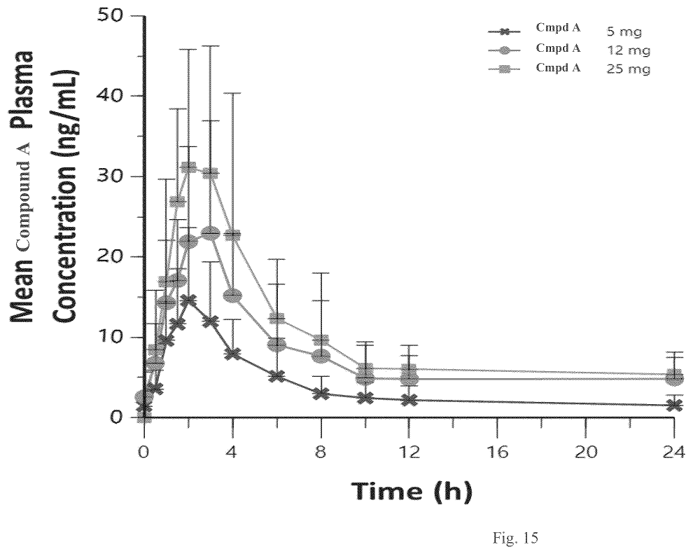

Mean concentration-time profile for Compound A in patients with NT2 is presented in FIG. 15 and key PK parameters are presented in Table 12b.

TABLE 12b
Summary of Plasma PK Parameters [Geometric mean (Geometric
CV %)] for Compound A By Dose - Part 5 (NT2)

| | 5 mg | | 12 mg | | 25 mg | |
|---|---|---|---|---|---|---|
| PK Parameters | (N = 9) | | (N = 9) | | (N = 9) | |
| Cₘₐₓ (ng/mL) | 13.4 | (53.3)¹ | 24.0 | (52.7)¹ | 38.0 | (34.5) |
| Tₘₐₓ (h) | 1.75 | (1.0-6.0)¹ | 2.0 | (1.0-4.0)¹ | 3.0 | (1.5-8.0) |
| AUC₀₋₂₄ | 78.1 | (58.6)¹ | 185.4 | (56.8)¹ | 230.8 | (38.0) |
| (ng*h/mL) | | | | | | |

Abbreviations: AUC₀₋₂₄ = area under the concentration-time curve from 0 to 24 hours, Cₘₐₓ = maximum plasma concentration, Tₘₐₓ = time to reach Cₘₐₓ, CV % = coefficient of variation percentage.
Data represented as geometric mean (geometric CV %). Tmax is represented as median (range).
¹n = 8

Systemic exposure to Compound A increased with dose in NT2 patients. A 5-fold change in dose resulted in approximately 3-fold change in AUC and Cₘₐₓ. Following oral administration, absorption of Compound A was moderate with median tₘₐₓ in the range of 1.75 hours to 3.0 hours across the dose range studied. Half-life was not derived as plasma concentrations were collected only for 24-hour period. At similar doses, PK exposure in NT2 population is comparable with that obtained in healthy volunteers.

### 4.18.6 Part 6 (IH Patients)

Eight subjects with IH were dosed in Part 6 (POC in IH) in a randomized 4-sequence 4-period cross-over design. Each dose of Compound A or matching placebo was separated by 2 days. A 14-day washout period preceded the treatment period and included discontinuation of medications used for the treatment of idiopathic hypersomnia. All 8 subjects underwent the washout period and received at least one dose of Compound A. All subjects completed all 4-way randomized crossover periods. (These data correspond with the data from IH subjects reported in Example 10.)

A majority of subjects were white (7 out of 8), all were non-Hispanic, and 7 out of 8 were females. Subjects ages ranged from 19 to 60 years, with a mean age of approximately 35 years. Subjects' BMI ranged from 20.9 to 32.3 kg/m², with a mean BMI of approximately 26.0 kg/m². Significant severity of idiopathic hypersomnia symptoms and excessive daytime sleepiness was observed at baseline (measured on Day −2, after the 2-week washout period). Idiopathic hypersomnia Severity Scale (IHSS) score ranged from 27 to 42, with a mean IHSS of 37.5. Epworth Sleepiness Scale (ESS) score ranged from 11 to 21, with a mean ESS of 14.8.

### Safety

Treatment with Compound A was well tolerated at all doses of Part 6 (IH). Eight subjects (100%) had at least one TEAE. All but one TEAEs were considered mild in severity (Table 11e). One subject experienced pollakiuria which was moderate in severity and resolved in 2 days. TEAEs reported as related to the study drug were mostly observed on 25 mg dose. No AEs leading to discontinuation occurred during this part of the study. There were no serious adverse events (SAEs) or deaths.

The incidence of TEAEs by dose level is presented in Table 11f. TEAEs that were assessed as related to Compound A and observed in more than 1 subject included pollakiuria, dizziness, and insomnia (including insomnia and middle insomnia). TEAEs that were assessed as related to Compound A and observed in only 1 subject included cold sweat, dysuria, feeling jittery, micturition urgency, palpitations, supraventricular extrasystoles, tachycardia and visual impairment (Table 11g).

TABLE 11e
Summary of Treatment Emergent Adverse Events (Safety Population) Part 6 POC IH

| | | Compound A | | | | |
|---|---|---|---|---|---|---|
| | | Compound A | Compound A | Compound A | | |
| | Placebo | 5 mg | 12 mg | 25 mg | Total | |
| | (N = 8) | (N = 8) | (N = 8) | (N = 8) | (N = 8) | |
| Number of Subjects | n (%) | n (%) | n (%) | n (%) | n (%) | |
| Any TEAE | 4 (50.0) | 6 (75.0) | 5 (62.5) | 7 (87.5) | 8 | (100) |
| TEAE by Highest | | | | | | |
| Severity^{a} | | | | | | |
| Mild | 4 (50.0) | 6 (75.0) | 5 (62.5) | 6 (75.0) | 7 | (87.5) |
| Moderate | 0 | 0 | 0 | 1 (12.5) | 1 | (12.5) |
| Severe | 0 | 0 | 0 | 0 | 0 | |
| TEAE by Highest | | | | | | |
| Relationship^{a} | | | | | | |
| Not Related | 1 (12.5) | 4 (50.0) | 2 (25.0) | 0 | 0 | |
| Related | 3 (37.5) | 2 (25.0) | 3 (37.5) | 7 (87.5) | 8 | (100) |

| | | | | | |
|---|---|---|---|---|---|
| AE Leading to Study | 0 | 0 | 0 | 0 | 0 |
| Drug Discontinuation | | | | | |
| Any SAEs | 0 | 0 | 0 | 0 | 0 |
| Death | 0 | 0 | 0 | 0 | 0 |

Abbreviations: AE = adverse event, TEAE = treatment emergent adverse event, SAE = serious adverse event, N = number of subjects dosed with each drug (on Day 1), n = number of subjects with adverse events in a group, IH = idiopathic hypersomnia, POC = proof-of-concept.
Percentages are based on the total number of subjects in the safety population in each column (N).
Adverse events are coded using MedDRA version 26.0.
TEAEs are defined as AEs that occur or worsen on or after the first dose of study drug.
If a subject experiences more than one adverse event in a category, the subject is counted only once in that category.
If a subject experiences the same adverse event at multiple dose levels, the subject will be counted once per dose level and once in total.
^{a}If a subject has multiple AEs, the highest severity is presented in summary by severity, and the closest relationship to study drug is presented in summary by relationship.

TABLE 11f
Summary of Treatment Emergent Adverse Events by
Preferred Term (Safety Population) Part 6 POC IH

| | | Compound A | | | | |
|---|---|---|---|---|---|---|
| | | Compound A | Compound A | Compound A | | |
| | Placebo | 5 mg | 12 mg | 25 mg | Total | |
| | (N = 8) | (N = 8) | (N = 8) | (N = 8) | (N = 8) | |
| Preferred Term | n (%) | n (%) | n (%) | n (%) | n (%) | |
| Subjects with at | 4 (50.0) | 6 (75.0) | 5 (62.5) | 7 (87.5) | 8 | (100) |
| least one TEAE | | | | | | |
| Pollakiuria | 1 (12.5) | 2 (25.0) | 2 (25.0) | 4 (50.0) | 5 | (62.5) |
| Dizziness | 0 | 2 (25.0) | 0 | 2 (25.0) | 4 | (50.0) |
| Headache | 0 | 1 (12.5) | 1 (12.5) | 1 (12.5) | 3 | (37.5) |
| Insomnia | 0 | 0 | 0 | 2 (25.0) | 2 | (25.0) |
| Middle insomnia | 0 | 1 (12.5) | 1 (12.5) | 1 (12.5) | 2 | (25.0) |
| Vessel puncture | 0 | 2 (25.0) | 0 | 0 | 2 | (25.0) |
| site bruise | | | | | | |
| Blood creatine | 0 | 0 | 1 (12.5) | 0 | 1 | (12.5) |
| phosphokinase | | | | | | |
| increased | | | | | | |
| Blood creatinine | 0 | 0 | 1 (12.5) | 0 | 1 | (12.5) |
| increased | | | | | | |
| Cold sweat | 0 | 0 | 0 | 1 (12.5) | 1 | (12.5) |
| Dysuria | 1 (12.5) | 0 | 1 (12.5) | 1 (12.5) | 1 | (12.5) |
| Epistaxis | 0 | 0 | 0 | 1 (12.5) | 1 | (12.5) |
| Feeling jittery | 0 | 0 | 0 | 1 (12.5) | 1 | (12.5) |
| Micturition urgency | 0 | 1 (12.5) | 0 | 1 (12.5) | 1 | (12.5) |
| Nausea | 1 (12.5) | 1 (12.5) | 1 (12.5) | 0 | 1 | (12.5) |
| Neck pain | 0 | 1 (12.5) | 0 | 0 | 1 | (12.5) |
| Palpitations | 0 | 0 | 0 | 1 (12.5) | 1 | (12.5) |
| Phlebitis | 0 | 0 | 1 (12.5) | 0 | 1 | (12.5) |
| Presyncope | 0 | 0 | 1 (12.5) | 0 | 1 | (12.5) |
| Skin irritation | 0 | 1 (12.5) | 0 | 0 | 1 | (12.5) |
| Steatorrhoea | 0 | 0 | 1 (12.5) | 0 | 1 | (12.5) |
| Supraventricular | 0 | 0 | 0 | 1 (12.5) | 1 | (12.5) |
| extrasystoles | | | | | | |
| Tachycardia | 1 (12.5) | 0 | 0 | 1 (12.5) | 1 | (12.5) |
| Urinary tract | 0 | 1 (12.5) | 0 | 0 | 1 | (12.5) |
| infection | | | | | | |
| Vessel puncture | 0 | 0 | 0 | 1 (12.5) | 1 | (12.5) |
| site pain | | | | | | |
| Visual impairment | 0 | 0 | 0 | 1 (12.5) | 1 | (12.5) |

| | | | | | |
|---|---|---|---|---|---|
| Dermatitis contact | 1 (12.5) | 0 | 0 | 0 | 0 |
| Vomiting | 1 (12.5) | 0 | 0 | 0 | 0 |

Abbreviations: AE = adverse event, TEAE = treatment emergent adverse event, N = number of subjects dosed with each drug (on Day 1), n = number of subjects with adverse events in a group, IH = idiopathic hypersomnia, POC = proof-of-concept.
Adverse events are coded using MedDRA version 26.0.
TEAEs are defined as AEs that occur or worsen on or after the first dose of study drug.
If a subject experiences more than one adverse event in a category, the subject is counted only once in that category. If a subject experiences the same adverse event at multiple dose levels, the subject will be counted once per dose level and once in total.

TABLE 11g
Summary of Drug Related Treatment Emergent Adverse Events
by Preferred Term (Safety Population) Part 6 POC IH

| | | Compound A | | | |
|---|---|---|---|---|---|
| | | Compound A | Compound A | Compound A | |
| | Placebo | 5 mg | 12 mg | 25 mg | Total |
| | (N = 8) | (N = 8) | (N = 8) | (N = 8) | (N = 8) |
| Preferred Term | n (%) | n (%) | n (%) | n (%) | n (%) |
| Subjects with at least one | 3 (37.5) | 2 (25.0) | 3 (37.5) | 7 (87.5) | 8 (100) |
| treatment-related TEAE | | | | | |
| Pollakiuria | 1 (12.5) | 2 (25.0) | 2 (25.0) | 4 (50.0) | 5 (62.5) |
| Dizziness | 0 | 0 | 0 | 2 (25.0) | 2 (25.0) |
| Insomnia | 0 | 0 | 0 | 2 (25.0) | 2 (25.0) |
| Middle insomnia | 0 | 1 (12.5) | 1 (12.5) | 1 (12.5) | 2 (25.0) |
| Cold sweat | 0 | 0 | 0 | 1 (12.5) | 1 (12.5) |
| Dysuria | 1 (12.5) | 0 | 1 (12.5) | 1 (12.5) | 1 (12.5) |
| Feeling jittery | 0 | 0 | 0 | 1 (12.5) | 1 (12.5) |
| Micturition urgency | 0 | 1 (12.5) | 0 | 1 (12.5) | 1 (12.5) |
| Palpitations | 0 | 0 | 0 | 1 (12.5) | 1 (12.5) |
| Supraventricular | 0 | 0 | 0 | 1 (12.5) | 1 (12.5) |
| extrasystoles | | | | | |
| Tachycardia | 1 (12.5) | 0 | 0 | 1 (12.5) | 1 (12.5) |
| Visual impairment | 0 | 0 | 0 | 1 (12.5) | 1 (12.5) |

Abbreviations: AE = adverse event, TEAE = treatment emergent adverse event, N = number of subjects dosed with each drug (on Day 1), n = number of subjects with adverse events in a group, IH = idiopathc hypersomnia, POC = proof-of-concept.
Adverse events are coded using MedDRA version 26.0.
TEAEs are defined as AEs that occur or worsen on or after the first dose of study drug.
If a subject experiences more than one adverse event in a category, the subject is counted only once in that category. If a subject experiences the same adverse event at multiple dose levels, the subject will be counted once per dose level and once in total.

Single doses of Compound A were generally well tolerated at all doses tested. All TEAEs were mild in severity with the exception of 1 patient experiencing a moderate pollakiuria at 25 mg dose. No deaths, SAEs, or TEAEs leading to discontinuation. The majority of TEAEs assessed as related to Compound A were observed in the 25 mg dose. Drug-related TEAEs observed in more than 1 subject included pollakiuria, dizziness, insomnia (including insomnia and middle insomnia). No drug-related treatment-emergent clinically meaningful changes were identified in laboratory values, vital signs, or ECGs. Overall safety profile in IH subjects is generally consistent with NT1 and healthy volunteer populations.

### Pharmacokinetics

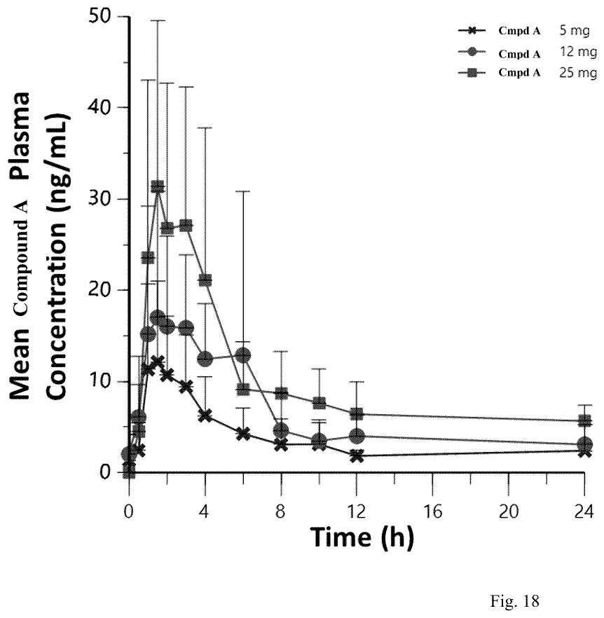

Mean concentration-time profile for Compound A in patients with IH is presented in FIG. 18 and key PK parameters are presented in Table 12c.

TABLE 12c
Summary of Plasma PK Parameters [Geometric mean (Geometric
CV %)] for Compound A By Dose - Part 6 (IH)

| | 5 mg | 12 mg | 25 mg |
|---|---|---|---|
| PK Parameters | (N = 8) | (N = 8) | (N = 8) |
| Cₘₐₓ (ng/mL) | 11.8 | 18.1 | 32.2 |
| | (65.0)¹ | (94.5)¹ | (71.2) |
| Tₘₐₓ (h) | 1.5 | 3.0 | 1.5 |
| | (1.0-2.0)¹ | (1.5-6.0)¹ | (1.0-4.0) |
| AUC₀₋₂₄ (ng*h/mL) | 78.9 | 155.5 | 229.5 |
| | (65.7)¹ | (103.3)¹ | (48.8) |

Abbreviations: AUC₀₋₂₄ = area under the concentration-time curve from 0 to 24 hours, Cₘₐₓ = maximum plasma concentration, Tₘₐₓ = time to reach Cₘₐₓ, CV % = coefficient of variation percentage.
Data represented as geometric mean (geometric CV %). Tmax is represented as median (range).
¹n = 7

Systemic exposure to Compound A increased with dose in IH patients. A 5-fold change in dose resulted in approximately 3-fold change in AUC and Cₘₐₓ. Following oral administration, absorption of Compound A was moderate with median tₘₐₓ in the range of 1.5 hours to 3.0 hours across the dose range studied. Half-life was not derived as plasma concentrations were collected only for 24-hour period. At similar doses, PK exposure in IH population is comparable with that obtained in healthy volunteers.

### 4.19.1 Summary of Efficacy (Part 4; NT1)

Evaluation of Compound A effects on average sleep latency across 4 timepoints of MWT (2-, 4-, 6-, and 8-hours post-dose) from 4 patients with NT1 has been conducted at pre-dose baseline series on Day −1, and each of the crossovers (Days 1, 3, 5 and 7), testing placebo, and active dose levels of 1, 3, and 8 mg in the patients with NT1 compared to placebo.

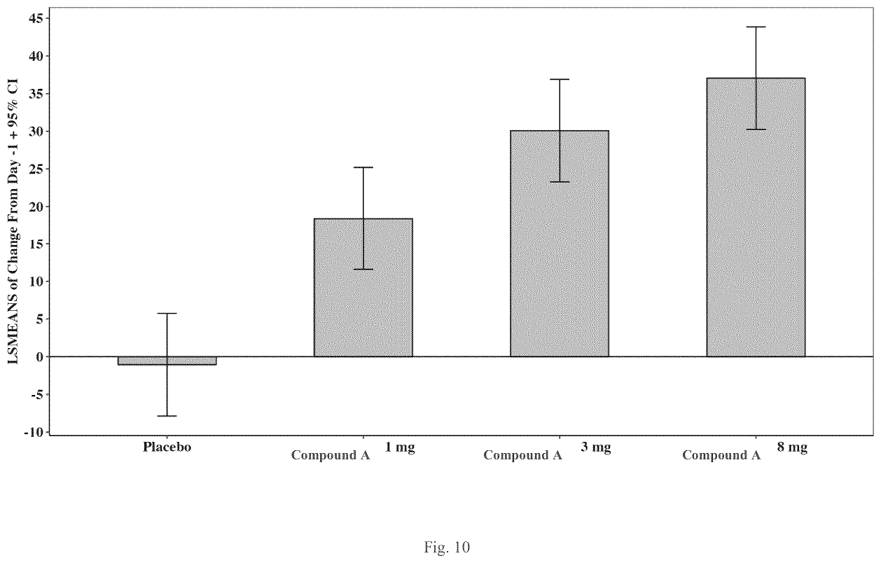

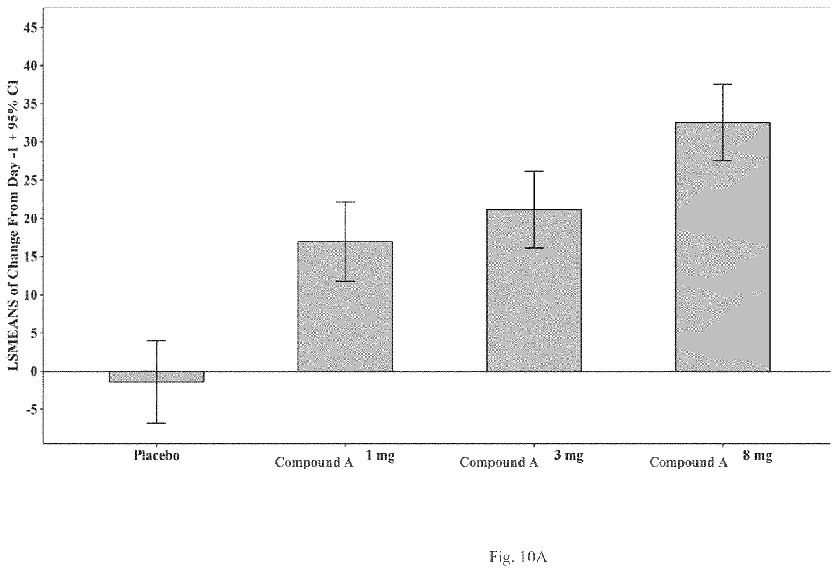

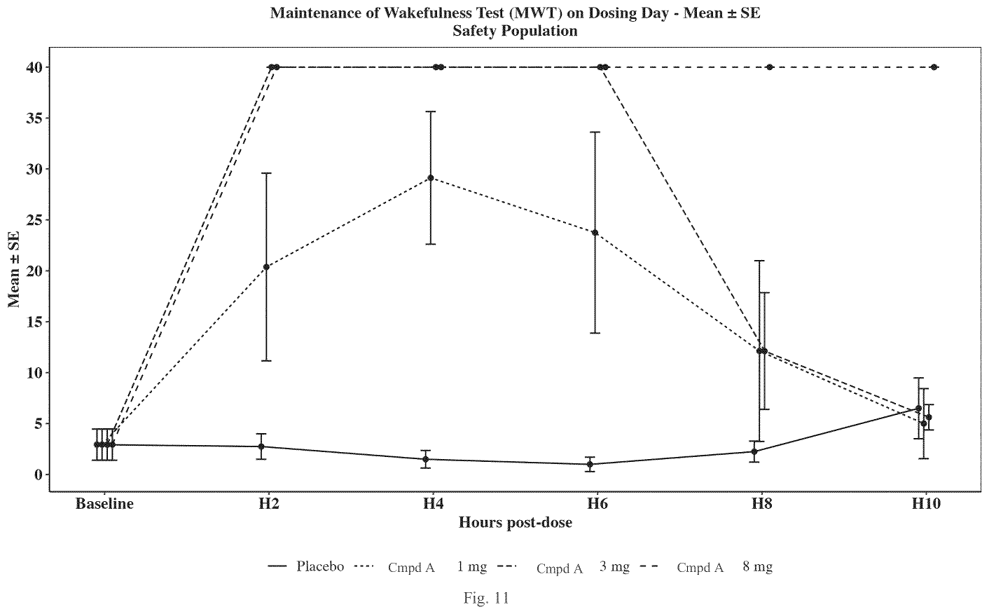

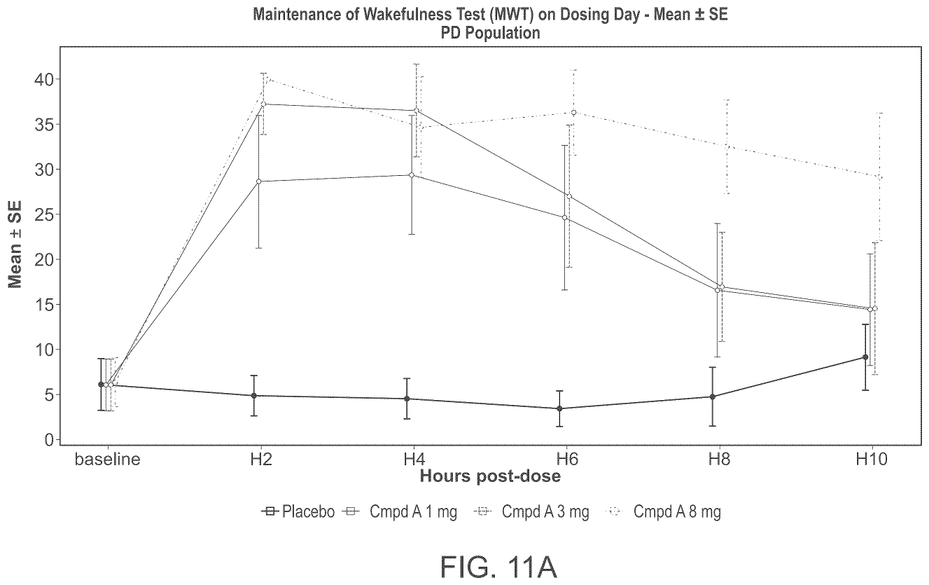

Analysis of change from baseline to post-dose showed a statistically significant improvement in sleep latency as measured by MWT in all doses tested (FIG. 10 and Table 13 below). By-timepoint analysis (FIG. 11) demonstrates dose-response in both the magnitude and durability of effect of Compound A: lowest dose (1 mg) appears to have clinically meaningful effect (average sleep latency improvement of 19.5 minutes), while 3 mg showed maximum effect over 2, 4 and 6-hour timepoints, and meaningful effect at 8 hours. Compound A dose of 8 mg had a maximum effect of 40 minutes at every timepoint, including 10 hours post-dose.

TABLE 13
Statistical Analysis of Change from Day −1 in Average Sleep Latency
on the Maintenance of Wakefulness Test (MWT) (PD Population) Part 4 POC NT1

| | | Compound A | | |
|---|---|---|---|---|
| | Placebo | 1 mg | 3 mg | 8 mg |
| Statistics^{a} | (N = 4) | (N = 4) | (N = 4) | (N = 4) |
| LSM | −1.06 | 18.41 | 30.09 | 37.06 |
| 95% CI of LSM | (−7.87, 5.74) | (11.60, 25.21) | (23.29, 36.90) | (30.26, 43.87) |
| LSM difference vs placebo | | 19.47 | 31.16 | 38.13 |
| 95% CI of LSM difference | | (9.82, 29.11) | (21.51, 40.80) | (28.48, 47.77) |
| p-value | | 0.0026 | 0.0002 | 0.0001 |

CI = confidence interval, LSM = least squares mean; MWT = Maintenance of Wakefulness Test, NT1 = narcolepsy type 1, PD = pharmacodynamic, POC = proof-of-concept, SE = standard error.
The PD Population is defined as all subjects in the Safety Population who have at least one baseline and at least one post-dose PD assessment.
^{a}Based on a mixed effect model of repeated measurement with the dose level and the period as fixed factors, and the average sleep latency on Day −1 is included as the baseline covariate. Average sleep latency is calculated as the average across the first four MWT sessions at 2, 4, 6, and 8 hours on Day −1 and at 2, 4, 6, and 8 hours post-dose on dosing days after a dosing time of approximately 9am.
Change from Day −1 value in average sleep latency at each dosing day is the dependent variable. A compound symmetry covariance matrix is used to model the within-subject errors. The Kenward-Roger approximation is used to estimate denominator degrees of freedom.

### 4.19a.1 Summary of Efficacy (Part 4; NT1)

Evaluation of Compound A effects on average sleep latency across 4 timepoints of MWT (2-, 4-, 6-, and 8-hours post-dose) from 10 patients with NT1 (including the 4 patients described above in 4.19.1) has been conducted at pre-dose baseline series on Day −1, and each of the crossovers (Days 1, 3, 5 and 7), testing placebo, and active dose levels of 1, 3, and 8 mg in the patients with NT1 compared to placebo.

Analysis of change from baseline to post-dose showed a statistically significant improvement in sleep latency as measured by MWT in all doses tested (FIG. 10A and Table 13a below). By-timepoint analysis (FIG. 11A) demonstrates dose-response in both the magnitude and durability of effect of Compound A.

TABLE 13a
Statistical Analysis of Change from Day −1 in Average Sleep Latency
on the Maintenance of Wakefulness Test (MWT) (PD Population) Part 4 POC NT1

| | | Compound A | | |
|---|---|---|---|---|
| | Placebo | 1 mg | 3 mg | 8 mg |
| Statistics^{a} | (N = 9) | (N = 9) | (N = 9) | (N = 10) |
| LSM | −1.42 | 16.95 | 21.15 | 32.55 |
| 95% CI of LSM | (−6.86, 4.01) | (11.77, 22.14) | (16.13, 26.17) | (27.57, 37.53) |
| LSM difference vs placebo | | 18.38 | 22.57 | 33.97 |
| 95% CI of LSM difference | | (10.95, 28.80) | (15.56, 29.58) | (26.72, 41.21) |
| p-value | | 0.0002 | 0.0001 | <0.0001 |

CI = confidence interval, LSM = least squares mean; MWT = Maintenance of Wakefulness Test, NT1 = narcolepsy type 1, PD = pharmacodynamic, POC = proof-of-concept, SE = standard error.
The PD Population is defined as all subjects in the Safety Population who have at least one baseline and at least one post-dose PD assessment.
^{a}Based on a mixed effect model of repeated measurement with the dose level and the period as fixed factors, and the average sleep latency on Day −1 is included as the baseline covariate. Average sleep latency is calculated as the average across the first four MWT sessions at 2, 4, 6, and 8 hours on Day −1 and at 2, 4, 6, and 8 hours post-dose on dosing days after a dosing time of approximately 9am.
Change from Day −1 value in average sleep latency at each dosing day is the dependent variable. An unstructured covariance matrix is used to model the within-subject errors.

### 4.19.2 Summary of Efficacy (Part 5; NT2)

Evaluation of Compound A effects on average sleep latency across 4 timepoints of MWT (2-, 4-, 6-, and 8-hours post-dose) has been conducted at each of the crossover periods (Days 1, 3, 5 and 7), testing placebo, and active dose levels of 5, 12, and 25 mg in the patients with NT2 compared to placebo. At pre-dose baseline on Day −1, MWT assessment was taken at similar times (2-, 4-, 6-, and 8-hours) as planned on dosing days assuming a dosing time of approximately 9 AM. Mean (SD) of baseline MWT average across the 4 time points on Day −1 was 14.31 (11.201), with high variability observed for NT2 subjects. (These data correspond with the data from NT2 subjects reported in Examples 9 and 10.)

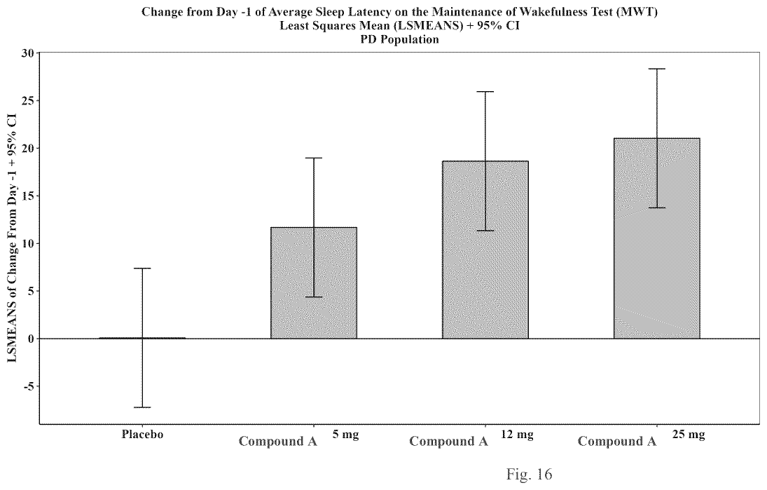

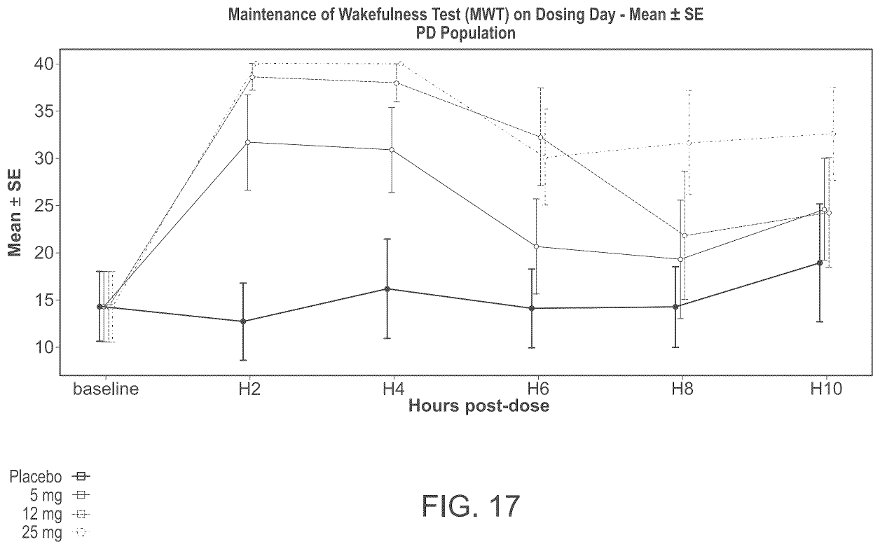

Analysis of change from baseline to post-dose showed a statistically significant improvement in sleep latency as measured by MWT in all doses tested (FIG. 16 and Tables 13b and 13c below). The least-squares mean differences in the change from baseline to post-dose as compared with placebo were 11.60 minutes (95% CI, 2.22 to 20.98, P=0.0178) on 5 mg dose, 18.55 minutes (95% CI, 9.17 to 27.93, P=0.0005) on 12 mg dose and 20.96 minutes (95% CI, 11.58 to 30.34, P=0.0001) on 25 mg dose. Notably, at the 12 mg and 25 mg doses, the observed mean MWT scores over an 8-hour period post-dose were ≥30 minutes. Overall, high variability was also observed on MWT on dosing days. A by-timepoint analysis of MWT change from the baseline (FIG. 17) which was the average of 4 assessments on Day −1 demonstrates a dose-response relationship in the magnitude and durability of effect of Compound A: effect of 12 mg and 25 mg was generally maintained across 2- to 8-hour time points with a single dose administration. In addition, a time-matched analysis of MWT change from time-matched baseline on Day −1 demonstrates similar patterns.

TABLE 13b
Statistical Analysis of Change from Day −1 in Average Sleep Latency
on the Maintenance of Wakefulness Test (MWT) (PD Population) Part 5 POC NT2

| | | Compound A | | |
|---|---|---|---|---|
| | Placebo | 5 mg | 12 mg | 25 mg |
| Statistics^{a} | (N = 9) | (N = 9) | (N = 9) | (N = 9) |
| LSM | 0.08 | 11.68 | 18.63 | 21.04 |
| 95% CI of LSM | (−7.22, 7.38) | (4.38, 18.98) | (11.33, 25.93) | (13.74, 28.34) |
| LSM difference vs placebo | | 11.60 | 18.55 | 20.96 |
| 95% CI of LSM difference | | (2.22, 20.98) | (9.17, 27.93) | (11.58, 30.34) |
| p-value | | 0.0178 | 0.0005 | 0.0001 |

CI = confidence interval, LSM = least squares mean; MWT = Maintenance of Wakefulness Test, NT2 = narcolepsy type 2, PD = pharmacodynamic(s), POC = proof-of-concept, SE = standard error.
The PD Population is defined as all subjects in the Safety Population who have at least one baseline and at least one post-dose PD assessment.
^{a}Based on a mixed effect model of repeated measurement with the dose level and the period as fixed factors, and the average sleep latency on Day −1 is included as the baseline covariate. Average sleep latency is calculated as the average across the first four MWT sessions at 2, 4, 6, and 8 hours on Day −1 and at 2, 4, 6, and 8 hours post-dose on dosing days after a dosing time of approximately 9am.
Change from Day −1 value in average sleep latency at each dosing day is the dependent variable. A compound symmetry covariance matrix is used to model the within-subject errors. The Kenward-Roger approximation is used to estimate denominator degrees of freedom.

TABLE 13c
Summary of Average Sleep Latency on the MWT (PD Population) Part 5 POC NT2

| | | Compound A | | |
|---|---|---|---|---|
| Parameter | Placebo | 5 mg | 12 mg | 25 mg |
| Statistics | (N = 9) | (N = 9) | (N = 9) | (N = 9) |
| Day −1^{a} | | | | |
| n | 9 | 9 | 9 | 9 |
| Mean (SD) | 14.31 (11.201) | 14.31 (11.201) | 14.31 (11.201) | 14.31 (11.201) |
| Median | 12.25 | 12.25 | 12.25 | 12.25 |
| Min, Max | 2.8, 32.9 | 2.8, 32.9 | 2.8, 32.9 | 2.8, 32.9 |
| Dosing Day in Each Period^{a} | | | | |
| n | 9 | 9 | 9 | 9 |
| Mean (SD) | 14.32 (11.949) | 25.95 (12.409) | 32.93 (9.836) | 35.44 (7.720) |
| Median | 13.50 | 26.63 | 40.00 | 40.00 |
| Min, Max | 1.8, 33.6 | 5.1, 40.0 | 15.1, 40.0 | 22.0, 40.0 |
| Change from Day −1 | | | | |
| n | 9 | 9 | 9 | 9 |
| Mean (SD) | 0.01 (11.371) | 11.65 (14.736) | 18.63 (10.892) | 21.14 (12.126) |
| Median | −1.88 | 8.50 | 19.50 | 17.25 |
| Min, Max | −19.4, 20.6 | −3.6, 37.3 | 7.1, 37.3 | 7.1, 37.3 |

Max = maximum, Min = minimum, MWT = Maintenance of Wakefulness Test, NT2 = narcolepsy type 2, PD = pharmacodynamic(s), POC-proof-of-concept, SD = standard deviation
The PD Population is defined as all subjects in the Safety Population who have at least 1 baseline and at least 1 post-dose PD assessment.
^{a}Average sleep latency is calculated as the average across the first 4 MWT sessions at 2, 4, 6, and 8 hours on Day −1 and at 2, 4, 6, and 8 hours post-dose on dosing days after a dosing time of approximately 9am.

### 4.19.2.1 Efficacy Endpoint: Karolinska Sleepiness Scale (KSS)

Effects of Compound A on sleep onset latency in the MWT were congruent with subjectively reported reduction in sleepiness on the Karolinska Sleepiness Scale (KSS). Analysis of change from Day −1 to post-dose showed a statistically significant improvement in KSS on 25 mg dose (Table 13d). The least-square mean differences in the change from Day −1 to post-dose as compared with placebo were −0.31 (95% CI, −1.21 to 0.58, P=0.4762) on 5 mg dose, −0.60 (95% CI, −1.50 to 0.29, P=0.1766) on 12 mg dose and −1.42 (95% CI, −2.31 to −0.52, P=0.0035) on 25 mg dose. A by-timepoint analysis demonstrated a dose-response relationship in the magnitude and durability of effect of Compound A.

TABLE 13d
Statistical Analysis of Change From Day −1 in Average
Karolinska Sleepiness Scale (PD Population) Part 5 POC NT2

| | | Compound A | | |
|---|---|---|---|---|
| | Placebo | 5 mg | 12 mg | 25 mg |
| Statistics^{a} | (N = 9) | (N = 9) | (N = 9) | (N = 9) |
| LSM | −0.37 | −0.68 | −0.97 | −1.78 |
| 95% CI of LSM | (−1.09, 0.35) | (−1.40, 0.04) | (−1.69, −0.25) | (−2.50, −1.06) |
| LSM difference vs placebo | | −0.31 | −0.60 | −1.42 |
| 95% CI of LSM difference | | (−1.21, 0.58) | (−1.50, 0.29) | (−2.31, −0.52) |
| p-value | | 0.4762 | 0.1766 | 0.0035 |

CI = confidence interval, LSM = least squares mean, KSS = Karolinska Sleepiness Scale, NT2 = narcolepsy type 2, PD = pharmacodynamic(s), POC-proof-of-concept, SE = standard error
The PD Population is defined as all subjects in the Safety Population who have at least 1 baseline and at least 1 post-dose PD assessment.
^{a}Based on a mixed effect model of repeated measurement with the dose level and the period as fixed factors, and the Pretreatment Day KSS score on (D −1) is included as the baseline covariate. The Pretreatment Day (D −1) KSS score is the average score of the time points (1, 2, 4, 6 and 8 hours) on Day −1. The Post-treatment KSS score is the average score of the time points (1, 2, 4, 6 and 8 hours) on dosing days.
Change from Day −1 value in Post-treatment KSS at each dosing day is the dependent variable. A compound symmetry covariance matrix is used to model the within-subject errors. The Kenward-Roger approximation is used to estimate denominator degrees of freedom.

All tested Compound A doses showed statistically significant and clinically meaningful improvement on average sleep onset latency via MWT test. Magnitude and durability of effect was dose dependent. At the 12 mg and 25 mg doses, the observed mean MWT scores over an 8-hour period post-dose were ≥30 minutes. Effects of Compound A on the Karolinska Sleepiness Scale were directionally consistent with the MWT results and statistically significant at the 25 mg dose.

### 4.19.3 Summary of Efficacy (Part 6; IH)

Evaluation of Compound A effects on average sleep latency across 4 timepoints of MWT (2-, 4-, 6-, and 8-hours post-dose) has been conducted at each of the crossover periods (Days 1, 3, 5 and 7), testing placebo, and active dose levels of 5, 12, and 25 mg in the patients with IH compared to placebo. At pre-dose baseline on Day −1, MWT assessment was taken at similar times (2-, 4-, 6-, and 8-hours) as planned on dosing days assuming a dosing time of approximately 9 AM. Mean (SD) of baseline MWT average across the 4 time points on Day −1 was 22.61 (9.294), with high variability observed for IH subjects. (These data correspond with the data from IH subjects reported in Example 10.)

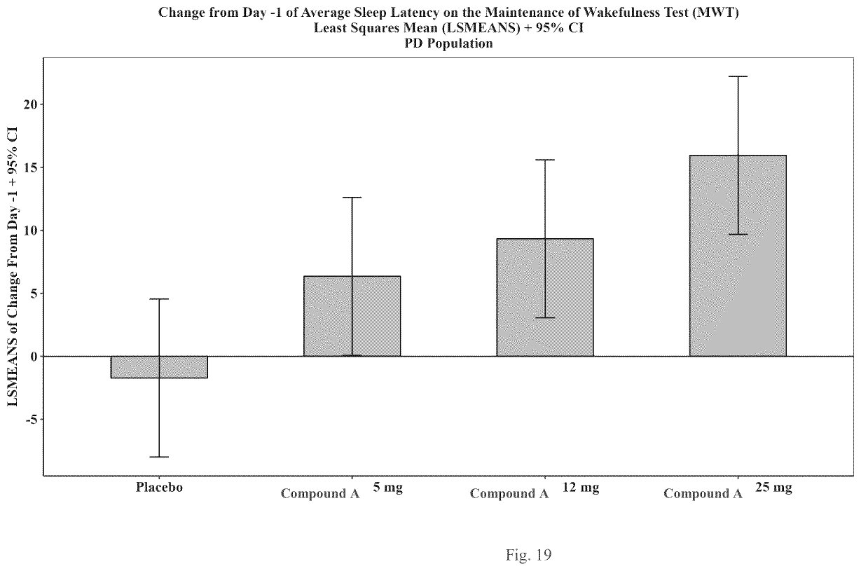

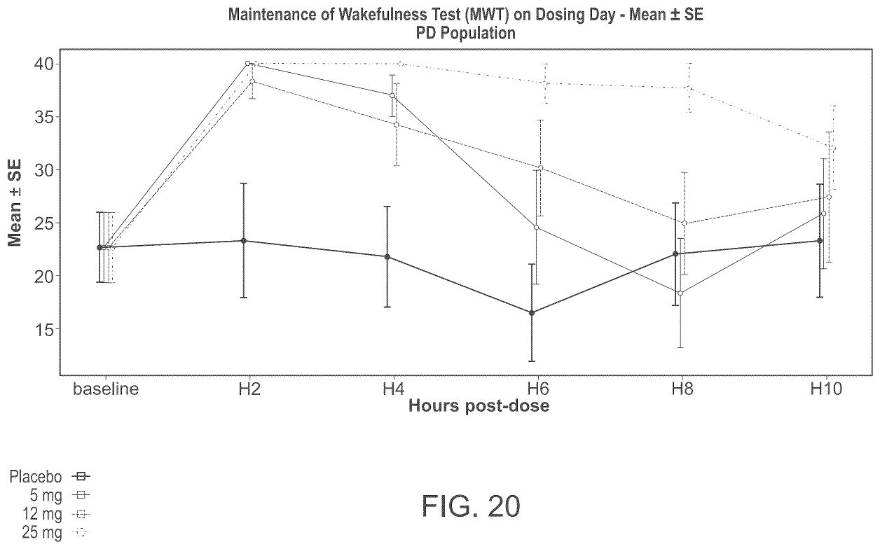

Analysis of change from baseline to post-dose showed a statistically significant improvement in sleep latency as measured by MWT in all doses tested (FIG. 19 and Tables 13e and 13f below). The least-squares mean differences in the change from baseline to post-dose as compared with placebo were 8.07 minutes (95% CI, 0.06 to 16.07, P=0.0484) on 5 mg dose, 11.05 minutes (95% CI, 3.04 to 19.05, P=0.0096) on 12 mg dose and 17.67 minutes (95% CI, 9.67 to 25.68, P=0.0002) on 25 mg dose. Notably, at the 12 mg and 25 mg doses, the observed mean MWT scores over an 8-hour period post-dose were ≥30 minutes. Overall, high variability was also observed on MWT on dosing days. A by-timepoint analysis of MWT change from the baseline (FIG. 20) which was the average of 4 assessments on Day −1 demonstrates a dose-response relationship in the magnitude and durability of effect of Compound A: effect of 25 mg was generally maintained across 2- to 8-hour time points with a single dose administration. In addition, a time-matched analysis of MWT change from time-matched baseline on Day −1 demonstrates similar patterns.

TABLE 13e
Statistical Analysis of Change from Day −1 in Average Sleep Latency
on the Maintenance of Wakefulness Test (MWT) (PD Population) Part 6 POC IH

| | | Compound A | | |
|---|---|---|---|---|
| | Placebo | 5 mg | 12 mg | 25 mg |
| Statistics^{a} | (N = 8) | (N = 8) | (N = 8) | (N = 8) |
| LSM | −1.72 | 6.35 | 9.33 | 15.95 |
| 95% CI of LSM | (−7.99, 4.55) | (0.08, 12.62) | (3.06, 15.60) | (9.68, 22.22) |
| LSM difference vs placebo | | 8.07 | 11.05 | 17.67 |
| 95% CI of LSM difference | | (0.06, 16.07) | (3.04, 19.05) | (9.67, 25.68) |
| p-value | | 0.0484 | 0.0096 | 0.0002 |

CI = confidence interval, LSM = least squares mean; MWT = Maintenance of Wakefulness Test, IH = idiopathic hypersomnia, PD = pharmacodynamic(s), POC = proof-of-concept, SE = standard error.
The PD Population is defined as all subjects in the Safety Population who have at least one baseline and at least one post-dose PD assessment.
^{a}Based on a mixed effect model of repeated measurement with the dose level and the period as fixed factors, and the average sleep latency on Day −1 is included as the baseline covariate. Average sleep latency is calculated as the average across the first four MWT sessions at 2, 4, 6, and 8 hours on Day −1 and at 2, 4, 6, and 8 hours post-dose on dosing days after a dosing time of approximately 9am.
Change from Day −1 value in average sleep latency at each dosing day is the dependent variable. A compound symmetry covariance matrix is used to model the within-subject errors. The Kenward-Roger approximation is used to estimate denominator degrees of freedom.

TABLE 13f
Summary of Average Sleep Latency on the MWT (PD Population) Part 6 POC IH

| | | Compound A | | |
|---|---|---|---|---|
| Parameter | Placebo | 5 mg | 12 mg | 25 mg |
| Statistics | (N = 8) | (N = 8) | (N = 8) | (N = 8) |
| Day −1^{a} | | | | |
| n | 8 | 8 | 8 | 8 |
| Mean (SD) | 22.61 (9.294) | 22.61 (9.294) | 22.61 (9.294) | 22.61 (9.294) |
| Median | 24.13 | 24.13 | 24.13 | 24.13 |
| Min, Max | 5.5, 33.8 | 5.5, 33.8 | 5.5, 33.8 | 5.5, 33.8 |
| Dosing Day in Each Period^{a} | | | | |
| n | 8 | 8 | 8 | 8 |
| Mean (SD) | 20.89 (12.359) | 28.96 (9.196) | 31.94 (7.814) | 38.56 (2.821) |
| Median | 19.63 | 29.13 | 32.69 | 40.00 |
| Min, Max | 3.2, 40.0 | 13.3, 40.0 | 15.9, 40.0 | 32.5, 40.0 |
| Change from Day −1 | | | | |
| n | 8 | 8 | 8 | 8 |
| Mean (SD) | −1.72 (12.109) | 6.35 (10.348) | 9.33 (11.468) | 15.95 (10.946) |
| Median | −0.90 | 4.75 | 7.00 | 15.31 |
| Min, Max | −22.0, 16.0 | −11.0, 24.4 | −9.3, 28.3 | −1.3, 34.5 |

Max = maximum, Min = minimum, MWT = Maintenance of Wakefulness Test, IH = idiopathic hypersomnia, PD = pharmacodynamic(s), POC = proof-of-concept, SD = standard deviation
The PD Population is defined as all subjects in the Safety Population who have at least 1 baseline and at least 1 post-dose PD assessment.
^{a}Average sleep latency is calculated as the average across the first 4 MWT sessions at 2, 4, 6, and 8 hours on Day −1 and at 2, 4, 6, and 8 hours post-dose on dosing days after a dosing time of approximately 9am.

### 4.19.3.1 Efficacy Endpoint: Karolinska Sleepiness Scale (KSS)

Effects of Compound A on sleep onset latency in the MWT were congruent with subjectively reported reduction in sleepiness on the Karolinska Sleepiness Scale (KSS). Analysis of change from Day −1 to post-dose showed a statistically significant improvement in KSS on 25 mg dose (Table 13g). The least-square mean differences in the change from Day −1 to post-dose as compared with placebo were −1.08 (95% CI, −2.24 to 0.09, P=0.0680) on 5 mg dose, −0.80 (95% CI, −1.96 to 0.36, P=0.1657) on 12 mg dose and −2.18 (95% CI, −3.34 to −1.01, P=0.0010) on 25 mg dose. A by-timepoint analysis generally demonstrated a dose-response relationship in the magnitude and durability of effect of Compound A.

TABLE 13g
Statistical Analysis of Change From Day −1 in Average
Karolinska Sleepiness Scale (PD Population) Part 6 POC IH

| | | Compound A | | |
|---|---|---|---|---|
| | Placebo | 5 mg | 12 mg | 25 mg |
| Statistics^{a} | (N = 8) | (N = 8) | (N = 8) | (N = 8) |
| LSM | −0.53 | −1.60 | −1.33 | −2.70 |
| 95% CI of LSM | (−1.57, 0.52) | (−2.65, −0.55) | (−2.37, −0.28) | (−3.75, −1.65) |
| LSM difference vs placebo | | −1.08 | −0.80 | −2.18 |
| 95% CI of LSM difference | | (−2.24, 0.09) | (−1.96, 0.36) | (−3.34, −1.01) |
| p-value | | 0.0680 | 0.1657 | 0.0010 |

CI = confidence interval, LSM = least squares mean, KSS = Karolinska Sleepiness Scale, IH = idiopathic hypersomnia, PD = pharmacodynamic(s), POC = proof-of-concept, SE = standard error
The PD Population is defined as all subjects in the Safety Population who have at least 1 baseline and at least 1 post-dose PD assessment.
^{a}Based on a mixed effect model of repeated measurement with the dose level and the period as fixed factors, and the Pretreatment Day KSS score on (D −1) is included as the baseline covariate. The Pretreatment Day (D −1) KSS score is the average score of the time points (1, 2, 4, 6 and 8 hours) on Day −1. The Post-treatment KSS score is the average score of the time points (1, 2, 4, 6 and 8 hours) on dosing days.
Change from Day −1 value in Post-treatment KSS at each dosing day is the dependent variable. A compound symmetry covariance matrix is used to model the within-subject errors. The Kenward-Roger approximation is used to estimate denominator degrees of freedom.

All tested Compound A doses showed statistically significant and clinically meaningful improvement on average sleep onset latency via MWT test. Magnitude and durability of effect was dose dependent. At the 12 mg and 25 mg doses, the observed mean MWT scores over an 8-hour period post-dose were ≥30 minutes. Effects of Compound A on the Karolinska Sleepiness Scale were consistent with the MWT results and statistically significant at the 25 mg dose.

### 4.20 Biomarker Data in Healthy Volunteers

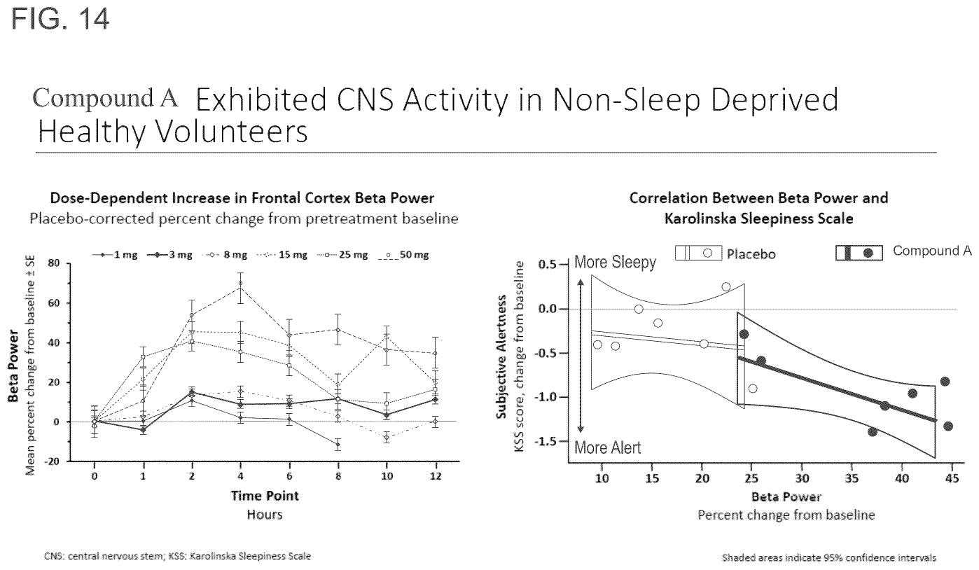

As discussed above in Example sections 4.10.3.2, 4.13.4, and 4.15.1, pharmacodynamic parameters in the SAD evaluation (e.g., changes from baseline in QTcF, power of qEEG frequency bands, KSS) were measured in non-sleep deprived healthy volunteers. FIG. 14 shows changes from baseline over time in quantitative EEG following single doses of Compound A. Beta band signal is shown on the vertical axis. Dose-dependent increases in beta band signal were observed, thereby demonstrating central nervous stem activity of Compound A during a resting wakeful state. Further, FIG. 14 also shows a correlation between post-dose beta band power and participants' subjective alertness scores on the Karolinska Sleepiness Scale (KSS). The dots indicate the average post-dose measurements at each timepoint and the shaded areas show the confidence intervals. The data points representing Compound A cluster in the area associated with both higher beta power and increased alertness relative to placebo, with little overlap between the two groups. Taken together, these data indicate that the increased beta power observed after a single dose of Compound A is associated with increased alertness, cognitive engagement, and quality of wakefulness during the day in non-sleep deprived healthy volunteers.

### Example 5—Compound A Powder in Capsule

The Compound A drug product was administered as a size 0, Swedish orange opaque, hydroxypropyl methylcellulose (HPMC) capsule with each capsule containing the active ingredient Compound A at target dosage strengths of 1 mg to 50 mg per capsule with no additional excipients. Compound A capsules were stored in high-density polyethylene (HDPE) bottles with child resistant closures (CRC). The composition of the Compound A drug product is presented in Table 14. This formulation was dosed in the powder-in-capsule (PIC) dosage forms referenced above in Example 4.

TABLE 14
Qualitative and Quantitative Composition
of Compound A Capsules

| | Quality | | Quantity |
|---|---|---|---|
| Ingredient | Standard | Function | per Capsule |
| Compound A | In-house | Drug substance | 1 mg to |
| | | | 50 mg |
| HPMC capsule | USP/NF | Capsule shell | 1 |
| | Ph. Eur. | | |
| | JP | | |

| | |
|---|---|
| Total Net Mass Range | 1 mg to |
| | 50 mg |

USP = United States Pharmacopeia, NF = National Formulary, Ph. Eur. = European Pharmacopeia, JP = Japanese Pharmacopeia

### Example 6—Compound A Tablet Formulation

The Compound A drug product was administered as a white, round, immediate-release (IR) tablet with each tablet containing the active ingredient Compound A, lactose monohydrate, microcrystalline cellulose, crospovidone, colloidal silicon dioxide, and magnesium stearate at a target dosage strength of 10 mg. Tablets were stored in white, induction-sealed, high-density polyethylene (HDPE) bottles with child-resistant closures (CRC). The composition of the Compound A drug product is presented in Table 15. This tablet formulation was dosed in the tablet dosage forms referenced above in Example 4 (Part 3-Relative Bioavailability with Food Effect study).

The tablets were compressed on a KG RoTab tablet press (KG-pharma GmbH & Co. KG, Germany) using a Turret speed of 20 rpm, four punches, a feeder speed of 50-100 rpm, a pre-compression force of 1 kN, and a cam size of 10 mm.

TABLE 15
Composition of Compound A Tablets

| | | Quality | Amount (mg) |
|---|---|---|---|
| Ingredient | Function | Standard | per Tablet |
| Compound A | Active | In-house | 10.00 |
| Lactose monohydrate | Diluent | USP/NF | 89.00 |
| | | Ph. Eur., JP | |
| Microcrystalline | Diluent | USP/NF | 94.00 |
| cellulose | | Ph. Eur., JP | |
| Crospovidone | Disintegrant | USP/NF | 4.00 |
| | | Ph. Eur., JP | |
| Colloidal silicon | Glidant | USP/NF | 1.00 |
| dioxide | | Ph. Eur., JP, IP | |
| Magnesium stearate | Lubricant | USP/NF | 2.00 |
| | | Ph. Eur., JP, BP, | |
| | | CHP | |
| Total Weight | — | — | 200.00 |

USP = United States Pharmacopeia, NF = National Formulary, Ph. Eur. = European Pharmacopeia, JP = Japanese, Pharmacopeia, IP = Indian Pharmacopeia, BP = British Pharmacopeia, CHP = Chinese Pharmacopeia

### Example 7—Oral Dosing of Compound A with Different Particle Sizes in Dogs

A non-GLP study was conducted in male Beagle dogs to compare the Compound A plasma exposure of powder-in-capsule (PIC) versus tablet formulation after the administration of single oral doses of micronized Compound A. Two different particle sizes were tested with the following objectives:
1. To compare the oral plasma exposure of micronized Compound A (12 micron Dv90 particle size) after the administration of 20 mg of Compound A powder-in-capsule vs 20 mg of Compound A in four tablets of 5 mg each.
2. To compare the oral plasma exposure of 12 micron and 32 micron Dv90 particle size of 20 mg micronized Compound A administered in four tablets of 5 mg each.
3. To evaluate the dose-linearity of Compound A by comparing the oral plasma exposure of Compound A following the oral administration of 5 mg (one tablet) versus 20 mg (four tablets) of Compound A.

The pharmacokinetic analysis of concentrations and concentration-time data are summarized in tabular form, below, using descriptive statistics. Concentration data were summarized according to nominal sampling times. Plasma concentrations below the lower limit of assay quantification (BLOQ) were not included in the computation of mean concentration values. Pharmacokinetic parameters for individual animals were estimated using the non-compartmental analysis (NCA) using Dotmatics Studies tool.

The following parameters were estimated whenever possible:

| | |
|---|---|
| Cₘₐₓ | Observed maximum concentration |
| Tₘₐₓ | Time to observed maximum concentration |
| AUC₀₋ₗₐₛₜ | Area under the concentration-time curve from time 0 to the |
| | time of the last measurable concentration, estimated by the |
| | linear trapezoidal rule |
| AUC_{0-∞} | Area under the concentration-time curve from time 0 |
| | extrapolated to infinity, calculated as AUC_{0-∞} = |
| | AUC₀₋ₗₐₛₜ + Cₗₐₛₜ / λ_{z} |

Where Cₗₐₛₜ is the last measurable concentration and λ_{z} is the terminal elimination rate constant estimated using log-linear regression of the terminal elimination phase of concentration-time curve

| | |
|---|---|
| t_{1/2} | Terminal elimination half-life, determined by ln(2)/λ_{z} |
| MRT | Mean residence time, Vₛₛ/CL |
| CL | Total body clearance, calculated as Dose/AUC_{0-∞} |
| Vₛₛ | Volume of distribution at steady state, calculated as |
| | (AUMC_{0-∞}/AUC_{0-∞}) × CL |

where AUMC_{0-∞} is the area under the moment curve from 0 to infinity CL, MRT, and Vₛₛ were calculated after IV administration only.

The absolute extravascular bioavailability (F %) were calculated using the following formula: F %= [(AUC_{0-∞(extravascular)}×Dose_{(intravenous(IV))})/(AUC_{0-∞(intravenous(IV))}×Dose_{(extravascular)})]×100 Where the “extravascular” represents any of subcutaneous (SC), intraperitoneal (IP) or oral (PO) route of administration.

The F % is calculated for each subject in case the same animals were administered with both intravenous and extravascular dose, then the average is reported with the standard error; otherwise, F % is calculated using the mean values without reporting a standard deviation in case the subjects of intravenous and extravascular profiles are different. Pharmacokinetic parameters were summarized by dosing group, route of administration, formulation, etc. using descriptive statistics.

### Results:

The pharmacokinetic parameters of micronized Compound A as PIC and 5 mg tablet using two particle sizes (12 micron and 32 micron Dv90) are summarized in Table 16. Compound A showed comparable plasma exposure between 20 mg powder in capsule and 20 mg in 4 tablets of 5 mg each of Compound A for both 12 micron and 32 micron Dv90 particle size with the F % ranging from 26% to 29%, Cₘₐₓ of 230 ng/mL to 270 ng/ml, AUC_{0-∞} of 1566 hr*ng/ml to 1728 hr*ng/ml, terminal t_{1/2} around 3 hr and Tₘₐₓ around 3 hr.

No dose-proportional increase of plasma exposure following the oral administration of 5 mg tablet/animal vs four 5 mg tablets/animal of Compound A (12 micron Dv90 particle size) was observed. Oral bioavailability (F %) was 55% and 29% for one tablet and four tablets, respectively.

TABLE 16
Pharmacokinetic Parameters (Means ± SD) of Compound A in Male Beagle Dogs
After Single Oral Doses of Compound A (Powder in Capsule (PIC) vs Tablet Formulation)

| | Dose | | | Cₘₐₓ | Tₘₐₓ | AUC₀₋ₗₐₛₜ | AUC_{0-∞} | t_{1/2} | |
|---|---|---|---|---|---|---|---|---|---|
| Route | (mg) | Food | Formulation | (ng/mL) | (hr) | (hr*ng/mL) | (hr*ng/mL) | (hr) | F %^{a} |
| PO | 20 | Fasted | Powder in Capsule | 259 ± 167 | 3.1 ± 0.9 | 1668 ± 1283 | 1728 ± 1275 | 4 ± 2 | 27 |
| | | | (12 micron Dv90) | | | | | | |
| PO | 5 | Fasted | 1x 5 mg Tablet A | 148 ± 83 | 2.8 ± 0.8 | 874 ± 806 | 914 ± 872 | 3 ± 2 | 55 |
| | | | (12 micron Dv90) | | | | | | |
| PO | 20 | Fasted | 4x 5 mg Tablet A | 230 ± 107 | 3 ± 1 | 1522 ± 962 | 1625 ± 1105 | 3.3 ± 0.4 | 29 |
| | | | (12 micron Dv90) | | | | | | |
| PO | 20 | Fasted | 4x 5 mg Tablet B | 270 ± 144 | 3 ± 1 | 1482 ± 1037 | 1566 ± 1167 | 2.8 ± 0.6 | 26 |
| | | | (32 micron Dv90) | | | | | | |

^{a}0.3 mg/kg IV bolus profile was used for the calculation of the absolute bioavailability
Abbreviations: NA = not available; AUC₀₋ₗₐₛₜ = Area Under the Curve from Time 0 to the Time of the Last Measurable Concentration; AUC_{0-∞} = Area Under the Curve from Time 0 Extrapolated to Infinity; Cₘₐₓ = Maximum Concentration; CL = Clearance; F = Oral Bioavailability; IV = Intravenous; PO = Oral Gavage; t_{1/2} = Terminal Elimination Half-life; Tₘₐₓ = Time to Observed Maximum Concentration; Vₛₛ = Volume of distribution at steady state; WFI = water for injection; PIC = powder in capsule.

Conclusion: Micronized Compound A showed comparable plasma exposure between 20 mg powder in capsule and 20 mg in 4 tablets of 5 mg each of Compound A for both 12 micron and 32 micron Dv90 particle size with the absolute F % ranging from 26% to 29%. No dose-proportional increase of plasma exposure following the oral administration of 5 mg tablet/animal vs four 5 mg tablets/animal of Compound A (12 micron Dv90 particle size) was observed. Oral bioavailability (F %) was 55% and 29% for one tablet and four tablets, respectively.

### Example 8—Compound A Crystalline Form

To a 50 mL vessel was added 2.04 g of Compound A. To this vessel was added 10 mL of dimethyl sulfoxide (DMSO). The reactor was set to heat to 80 degrees C. at 0.1 degrees C./min with overhead stirring at 350 rpm. At 80 degrees C., a clear solution was obtained, and the reactor was set to cool to 60 degrees C. at 0.1 degrees C./min. At 62 degrees C., 1.0 mL of water was added with no precipitation noted. At 60 degrees C., precipitation was noted, and the reactor was set to cool to 20 degrees C. at 0.05 degrees C./min overnight. After stirring at 20 degrees C. overnight, the reactor was heated to 50 degrees C. at 1.0 degrees C./min and held for 30 minutes to dissolve fines. Reactor was again cooled to 20 degrees C. at 0.1 degrees C./min. At 20 degrees C., 2.5 mL of water was added to bring the total composition to 75/25 (v/v) DMSO/water. An additional 6.5 mL of water was added to bring the final composition to 50/50 (v/v) DMSO/water. Solids were isolated via filtration and washed with 10 mL of water before being placed in a vacuum oven to dry overnight.

This crystalline form of Compound A was used/dosed in each of the Examples 1-7 provided herein.

The PXRD diffractogram for the Compound A crystalline form provided herein is shown in FIG. 12. Table 17 also shows PXRD data.

TABLE 17

| | Angle |
|---|---|
| Peak Number | (degrees 2θ) |
| 1 | 8.95 |
| 2 | 10.44 |
| 3 | 11.33 |
| 4 | 12.51 |
| 5 | 13.81 |
| 6 | 15.21 |
| 7 | 16.52 |
| 8 | 17.35 |
| 9 | 17.92 |
| 10 | 20.95 |
| 11 | 22.54 |
| 12 | 24.94 |
| 13 | 27.93 |

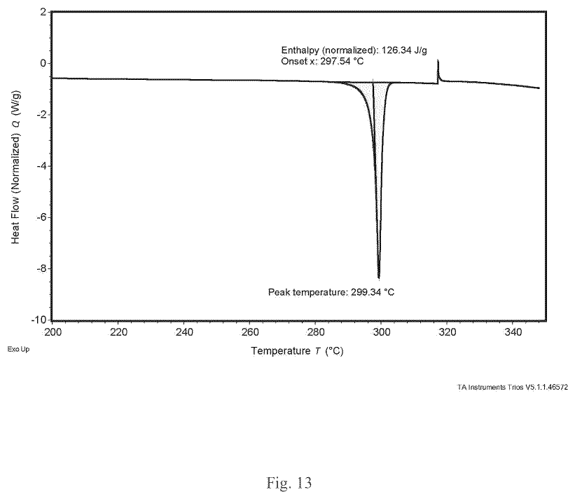

DSC analysis of the Compound A crystalline form was performed by ramping 10 degrees C. per minute from 20 degrees to 350 degrees C. The DSC thermogram is depicted in FIG. 13. An endotherm is observed with an onset temperature of 297.5 degrees C. and a peak temperature of 299.3 degrees C.

### Example 9—Compound A in Patients with Narcolepsy Type 2: An Initial Proof of Concept Phase 1b Study

This example summarizes data relating to Part 5 (NT2 subjects) of the clinical study described above in Example 4. Compound A is a potent, centrally active, orally bioavailable, and highly selective orexin 2 receptor agonist being developed as a once-daily treatment for narcolepsy. Results from single- and multiple-dose pharmacokinetic and safety studies in healthy volunteers, and initial proof of concept data in patients with narcolepsy type 1 (NT1), are reported in Example 4 above. Provided here are safety and pharmacodynamic results in patients with narcolepsy type 2 (NT2).

Methods: This randomized, double-blind, phase 1b study assessed the safety, tolerability, and pharmacodynamics of Compound A. Patients with NT2 in Australia and the US received single doses of 5, 12, and 25 mg Compound A and matching placebo in a 4-way randomized crossover design following washout from their current narcolepsy medications. Safety assessments included adverse events (AEs), vital signs, clinical laboratory testing of blood and urine, and electrocardiograms (ECG). The key pharmacodynamic assessment was mean sleep latency on the Maintenance of Wakefulness Test (MWT).

Results: In patients with NT2 (N=9), there were no serious AEs, and no patient discontinued due to any AE. Adverse events related to study drug and occurring in >1 patient were pollakiuria, insomnia, and dizziness. All AEs were mild except 1 case of moderate pollakiuria. No treatment-emergent, clinically meaningful changes from baseline were identified in laboratory values, vital signs, or ECGs. Compound A increased mean sleep latency versus placebo over 8 hours post-dose, as assessed by the estimated least square mean difference in the change from baseline, by 11.6 minutes (5 mg, p<0.05), 18.6 minutes (12 mg, p<0.001), and 21.0 minutes (25 mg, p<0.001). The observed mean sleep latencies at doses of 12 and 25 mg were ≥30 minutes.

Conclusions: Compound A was generally well-tolerated among patients with NT2. Single doses of Compound A at 5, 12, and 25 mg led to statistically significant, clinically meaningful improvements in sleep latency. At the 12 and 25 mg doses, the mean sleep latencies achieved were within the reported range for healthy individuals.

### Example 10—Results from Clinical Study of Compound A Demonstrating Improved Wakefulness in Patients with Narcolepsy Type 2 and Idiopathic Hypersomnia

This example summarizes data relating to Part 5 (Narcolepsy Type 2) and Part 6 (Idiopathic Hypersomnia) of the clinical study described above in Example 4. Compound A data demonstrated clinically meaningful and statistically significant improvements from baseline in mean sleep latency on the Maintenance of Wakefulness Test (MWT) compared to placebo at all doses tested. Compound A was generally well tolerated in both patient populations at all doses tested. Methods: The phase 1b NT2 (n=9) and IH (n=8) study cohorts evaluated the safety, tolerability, pharmacokinetics and pharmacodynamics of Compound A via once-daily, single, oral administration. Participants were randomized to a four-way crossover study in which each participant received 5 mg, 12 mg and 25 mg of Compound A, and placebo, with washout periods between each treatment.

Results from Each Cohort are as Follows:

### Narcolepsy Type 2:

In the nine patients with NT2, treatment with Compound A demonstrated improved wakefulness compared to placebo at all doses tested, with a clear dose response. Prior to treatment with Compound A, these patients had baseline sleep latencies ranging from 3 to 33 minutes, with a mean sleep latency of 14 minutes at baseline. Treatment with Compound A resulted in statistically significant and clinically meaningful improvements in sleep latency in patients with NT2, with a mean change from baseline versus placebo of 12 minutes at the 5 mg dose (p<0.05), 19 minutes at the 12 mg dose (p<0.001), and 21 minutes at the 25 mg dose (p<0.001) (least squares mean difference). Placebo treatment resulted in no change in mean sleep latency. At the 12 mg and 25 mg doses, the observed mean MWT scores over an eight-hour period post-dose were within the reported normal range for healthy individuals (See Krahn L E, Arand D L, Avidan A Y, et al. Recommended protocols for the Multiple Sleep Latency Test and the Maintenance of Wakefulness Test in adults: guidance from the American Academy of Sleep Medicine. J. Clin. Sleep Med. 2021; 17(12):2489-2498.)

Compound A was generally well tolerated across all doses tested in participants with NT2. All treatment-emergent adverse events (TEAEs) were transient and self-resolving. TEAEs were mild in severity, with the exception of one moderate case of pollakiuria at the highest dose (25 mg). AEs observed in >1 participant with NT2 and deemed to be related to study drug were pollakiuria, insomnia and dizziness. One mild, transient occurrence of photophobia was reported in a single patient at the 25 mg dose. The occurrence self-resolved within two hours of onset.

There were no serious AEs or AEs leading to discontinuation in patients with NT2. Additionally, there were not clinically meaningful, treatment-emergent changes in hepatic and renal parameters, vital signs, or electrocardiogram (ECG) parameters.

### Idiopathic Hypersomnia:

In the eight patients with IH, treatment with Compound A demonstrated improved wakefulness compared to placebo at all doses tested, with a clear dose response. Prior to treatment with Compound A, these patients had baseline sleep latencies ranging from 6 to 34 minutes, with a mean sleep latency of 23 minutes at baseline. Treatment with Compound A resulted in statistically significant and clinically meaningful improvements in sleep latency in patients with IH, with a mean change from baseline versus placebo of 8 minutes at the 5 mg dose (p<0.05), 11 minutes at the 12 mg dose (p<0.01), and 18 minutes at the 25 mg dose (p<0.001) (least squares mean difference). Placebo treatment resulted in a two-minute reduction in mean sleep latency. At the 12 mg and 25 mg doses, the observed mean MWT scores over an eight-hour period post-dose were within the reported normal range for healthy individuals. (See Krahn L E, Arand D L, Avidan A Y, et al. Recommended protocols for the Multiple Sleep Latency Test and the Maintenance of Wakefulness Test in adults: guidance from the American Academy of Sleep Medicine. J. Clin. Sleep Med. 2021; 17(12):2489-2498.)

Compound A was generally well tolerated across all doses tested in participants with IH. All TEAEs were transient and self-resolving. TEAEs were mild in severity, with the exception of one moderate case of pollakiuria at the highest dose (25 mg). AEs observed in >1 participant and deemed to be related to study drug were pollakiuria, insomnia and dizziness. One mild, transient occurrence of visual disturbance was reported in a single patient at the 25 mg dose. The occurrence self-resolved approximately one hour after onset.

There were no serious AEs or AEs leading to discontinuation. Additionally, there were not clinically meaningful, treatment-emergent changes in hepatic and renal parameters, vital signs, or ECG parameters.

### Embodiments

1. A method for treating narcolepsy in a subject in need thereof, comprising administering to the subject an effective amount of Compound A or a pharmaceutically acceptable salt thereof, wherein Compound A or a pharmaceutically acceptable salt thereof is administered orally once or more per day.

2. The method of embodiment 1, wherein Compound A or a pharmaceutically acceptable salt is administered orally once per day.

3, The method of embodiment 1 or 2, wherein Compound A is administered at a daily dose of from about 0.5 mg to about 50 mg.

4. The method of embodiment 1 or 2, wherein Compound A is administered at a daily dose of from about 1 mg to about 25 mg.

5. The method of embodiment 1 or 2, wherein Compound A is administered at a daily dose of from about 8 mg to about 25 mg.

6. The method of embodiment 1 or 2, wherein Compound A is administered at a daily dose of from about 1 mg to about 10 mg.

7. The method of embodiment 1 or 2, wherein Compound A is administered at a daily dose of from about 2 mg to about 6 mg.

8. The method of any of embodiments 1-7, wherein the Compound A has a volume based particle size with a Dv90 of between about 5 and about 200 microns as determined by a light scattering technique.

9. The method of any of embodiments 1-7, wherein the Compound A has a volume based particle size with a Dv90 of between about 5 and about 100 microns as determined by a light scattering technique.

10. The method of any of embodiments 1-7, wherein the Compound A has a volume based particle size with a Dv90 of between about 5 and about 50 microns as determined by a light scattering technique.

11. The method of any of embodiments 1-10, wherein the Compound A provides a Cₘₐₓ of Compound A of not more than about 50 ng/mL.

12. The method of any of embodiments 1-10, wherein the Compound A provides a Cₘₐₓ of Compound A of not more than about 40 ng/mL.

13. The method of any of embodiments 1-10, wherein the Compound A provides a Cₘₐₓ of Compound A of not more than about 35 ng/ml.

14. The method of any of embodiments 1-10, wherein the Compound A provides a Cₘₐₓ of

Compound A of not more than about 30 ng/mL.

15. The method of any of embodiments 1-10, wherein the Compound A provides a Cₘₐₓ of Compound A of not more than about 25 ng/ml.

16. The method of any of embodiments 1-10, wherein the Compound A provides a Cₘₐₓ of Compound A of not more than about 20 ng/mL.

17. The method of any of embodiments 1-10, wherein the Compound A provides a Cₘₐₓ of Compound A of not more than about 15 ng/mL.

18. The method of any of embodiments 1-10, wherein the Compound A provides a Cₘₐₓ of Compound A of not more than about 10 ng/mL.

19. The method of any of embodiments 1-10, wherein the Compound A provides a Cₘₐₓ of

Compound A of not more than about 5 ng/mL.

20. The method of any of embodiments 1-19, wherein the Compound A provides a Cₘₐₓ of Compound A of between about 5 ng/ml and about 35 ng/mL and a half-life (t_{1/2}) of Compound A between about 4 and about 12 hours.

21. The method of any of embodiments 1-19, wherein the Compound A provides a Cₘₐₓ of Compound A of between about 5 ng/mL and about 35 ng/mL and a half-life (t_{1/2}) of Compound A between about 6 and about 12 hours.

22. The method of any of embodiments 1-19, wherein the Compound A provides a Cₘₐₓ of Compound A of between about 5 ng/ml and about 35 ng/ml and a half-life (t_{1/2}) of Compound A between about 8 and about 12 hours.

23. The method of any of embodiments 1-19, wherein the Compound A provides a Cₘₐₓ of Compound A of between about 5 ng/mL and about 35 ng/ml and a half-life (t_{1/2}) of Compound A between about 8 and about 10 hours.

24. The method of any of embodiments 1-23, wherein the Compound A provides a first plasma concentration peak of Compound A of between about 5 ng/ml and about 50 ng/ml within about 4 hours and a second plasma concentration peak between about 8 and about 24 hours following oral administration.

25. The method of any of embodiments 1-24, wherein the plasma concentration for Compound A following oral administration is about 3.0 ng/mL or more at 2 hours after administration and about 1.0 ng/mL or less at 12 hours after administration.

26. The method of any of embodiments 1-24, wherein the plasma concentration for Compound A following oral administration is about 3.0 ng/mL or more at 2 hours after administration and about 1.5 ng/mL or less at 12 hours after administration.

27. The method of any of embodiments 1-24, wherein the plasma concentration for Compound A following oral administration is about 4.0 ng/mL or more at 2 hours after administration and about 1.5 ng/mL or less at 12 hours after administration.

28. The method of any of embodiments 1-24, wherein the plasma concentration for Compound A following oral administration is about 5.0 ng/mL or more at 2 hours after administration and about 1.5 ng/mL or less at 12 hours after administration.

29. The method of any of embodiments 1-24, wherein the plasma concentration for Compound A following oral administration is about 10.0 ng/mL or more at 2 hours after administration and about 2.0 ng/mL or less at 12 hours after administration.

30. The method of any of embodiments 1-24, wherein the plasma concentration for Compound A following oral administration is about 15.0 ng/mL or more at 2 hours after administration and about 2.0 ng/mL or less at 12 hours after administration.

31. The method of any of embodiments 1-24, wherein the plasma concentration for Compound A following oral administration is about 20.0 ng/ml or more at 2 hours after administration and about 2.0 ng/mL or less at 12 hours after administration.

32. The method of any of embodiments 1-24, wherein the plasma concentration for Compound A following oral administration is about 20.0 ng/mL or more at 2 hours after administration and about 4.0 ng/mL or less at 12 hours after administration.

33. A method for treating narcolepsy type 1 in a subject in need thereof, comprising administering to the subject an effective amount of Compound A or a pharmaceutically acceptable salt thereof, wherein Compound A or a pharmaceutically acceptable salt thereof is administered orally once or more per day.

34. The method of embodiment 33, wherein Compound A or a pharmaceutically acceptable salt is administered orally once per day.

35. The method of embodiment 33 or 34, wherein Compound A is administered at a daily dose of from about 1 mg to about 10 mg.

36. The method of embodiment 33 or 34, wherein Compound A is administered at a daily dose of from about 2 mg to about 6 mg.

37. The method of any of embodiments 33-36, wherein the Compound A has a volume based particle size with a Dv90 of between about 5 and about 200 microns as determined by a light scattering technique.

38. The method of any of embodiments 33-36, wherein the Compound A has a volume based particle size with a Dv90 of between about 5 and about 100 microns as determined by a light scattering technique.

39. The method of any of embodiments 33-36, wherein the Compound A has a volume based particle size with a Dv90 of between about 5 and about 50 microns as determined by a light scattering technique.

40. The method of any of embodiments 33-39, wherein the Compound A provides a Cₘₐₓ of Compound A of not more than about 25 ng/mL.

41. The method of any of embodiments 33-39, wherein the Compound A provides a Cₘₐₓ of Compound A of not more than about 20 ng/mL.

42. The method of any of embodiments 33-39, wherein the Compound A provides a Cₘₐₓ of

Compound A of not more than about 15 ng/mL.

43. The method of any of embodiments 33-39, wherein the Compound A provides a Cₘₐₓ of Compound A of not more than about 10 ng/mL.

44. The method of any of embodiments 33-39, wherein the Compound A provides a Cₘₐₓ of Compound A of not more than about 5 ng/mL.

45. The method of any of embodiments 33-44, wherein the Compound A provides a Cₘₐₓ of Compound A of between about 5 ng/ml and about 35 ng/ml and a half-life (t_{1/2}) of Compound A between about 4 and about 12 hours.

46. The method of any of embodiments 33-44, wherein the Compound A provides a Cₘₐₓ of Compound A of between about 5 ng/mL and about 35 ng/ml and a half-life (t_{1/2}) of Compound A between about 6 and about 12 hours.

47. The method of any of embodiments 33-44, wherein the Compound A provides a Cₘₐₓ of Compound A of between about 5 ng/ml and about 35 ng/ml and a half-life (t_{1/2}) of Compound A between about 8 and about 12 hours.

48. The method of any of embodiments 33-44, wherein the Compound A provides a Cₘₐₓ of Compound A of between about 5 ng/ml and about 35 ng/ml and a half-life (t_{1/2}) of Compound A between about 8 and about 10 hours.

49. The method of any of embodiments 33-48, wherein the Compound A provides a first plasma concentration peak of Compound A of between about 5 ng/mL and about 50 ng/ml within about 4 hours and a second plasma concentration peak between about 8 and about 24 hours following oral administration.

50. The method of any of embodiments 33-48, wherein the plasma concentration for Compound A following oral administration is about 3.0 ng/ml or more at 2 hours after administration and about 1.0 ng/mL or less at 12 hours after administration.

51. The method of any of embodiments 33-48, wherein the plasma concentration for Compound A following oral administration is about 3.0 ng/mL or more at 2 hours after administration and about 1.5 ng/mL or less at 12 hours after administration.

52. The method of any of embodiments 33-48, wherein the plasma concentration for Compound A following oral administration is about 4.0 ng/mL or more at 2 hours after administration and about 1.5 ng/ml or less at 12 hours after administration.

53. The method of any of embodiments 33-48, wherein the plasma concentration for Compound A following oral administration is about 5.0 ng/mL or more at 2 hours after administration and about 1.5 ng/mL or less at 12 hours after administration.

54. The method of any of embodiments 33-48, wherein the plasma concentration for Compound A following oral administration is about 10.0 ng/ml or more at 2 hours after administration and about 2.0 ng/mL or less at 12 hours after administration.

55. The method of any of embodiments 33-48, wherein the plasma concentration for Compound A following oral administration is about 15.0 ng/ml or more at 2 hours after administration and about 2.0 ng/mL or less at 12 hours after administration.

56. The method of any of embodiments 33-48, wherein the plasma concentration for Compound A following oral administration is about 20.0 ng/ml or more at 2 hours after administration and about 2.0 ng/mL or less at 12 hours after administration.

57. A method for treating narcolepsy type 2 in a subject in need thereof, comprising administering to the subject an effective amount of Compound A or a pharmaceutically acceptable salt thereof, wherein Compound A or a pharmaceutically acceptable salt thereof is administered orally once or more per day.

58. The method of embodiment 57, wherein Compound A or a pharmaceutically acceptable salt is administered orally once per day.

59. The method of embodiment 57 or 58, wherein Compound A is administered at a daily dose of from about 0.5 mg to about 50 mg.

60. The method of embodiment 57 or 58, wherein Compound A is administered at a daily dose of from about 1 mg to about 25 mg.

61. The method of embodiment 57 or 58, wherein Compound A is administered at a daily dose of from about 8 mg to about 25 mg.

62. The method of any of embodiments 57-61, wherein the Compound A has a volume based particle size with a Dv90 of between about 5 and about 200 microns as determined by a light scattering technique.

63. The method of any of embodiments 57-61, wherein the Compound A has a volume based particle size with a Dv90 of between about 5 and about 100 microns as determined by a light scattering technique.

64. The method of any of embodiments 57-61, wherein the Compound A has a volume based particle size with a Dv90 of between about 5 and about 50 microns as determined by a light scattering technique.

65. The method of any of embodiments 57-64, wherein the Compound A provides a Cₘₐₓ of Compound A of not more than about 50 ng/ml.

66. The method of any of embodiments 57-64, wherein the Compound A provides a Cₘₐₓ of Compound A of not more than about 40 ng/ml.

67. The method of any of embodiments 57-64, wherein the Compound A provides a Cₘₐₓ of Compound A of not more than about 35 ng/mL.

68. The method of any of embodiments 57-67, wherein the Compound A provides a Cₘₐₓ of Compound A of between about 5 ng/ml and about 50 ng/mL and a half-life (t_{1/2}) of Compound A between about 4 and about 12 hours.

69. The method of any of embodiments 57-67, wherein the Compound A provides a Cₘₐₓ of Compound A of between about 10 ng/ml and about 50 ng/ml and a half-life (t_{1/2}) of Compound A between about 6 and about 12 hours.

70. The method of any of embodiments 57-67, wherein the Compound A provides a Cₘₐₓ of Compound A of between about 15 ng/ml and about 50 ng/ml and a half-life (t_{1/2}) of Compound A between about 8 and about 12 hours.

71. The method of any of embodiments 57-67, wherein the Compound A provides a Cₘₐₓ of Compound A of between about 15 ng/ml and about 50 ng/ml and a half-life (t_{1/2}) of Compound A between about 8 and about 10 hours.

72. The method of any of embodiments 57-71, wherein the Compound A provides a first plasma concentration peak of Compound A of between about 5 ng/ml and about 50 ng/ml within about 4 hours and a second plasma concentration peak between about 8 and about 24 hours following oral administration.

73. The method of any of embodiments 57-72, wherein the plasma concentration for Compound A following oral administration is about 15.0 ng/mL or more at 2 hours after administration and about 2.0 ng/mL or less at 12 hours after administration.

74. The method of any of embodiments 57-72, wherein the plasma concentration for Compound A following oral administration is about 20.0 ng/mL or more at 2 hours after administration and about 3.0 ng/mL or less at 12 hours after administration.

75. The method of any of embodiments 57-72, wherein the plasma concentration for Compound A following oral administration is about 20.0 ng/mL or more at 2 hours after administration and about 4.0 ng/mL or less at 12 hours after administration.

76. A method for treating excessive daytime sleepiness or cataplexy in a subject with narcolepsy, comprising administering to the subject an effective amount of Compound A or a pharmaceutically acceptable salt thereof, wherein Compound A or a pharmaceutically acceptable salt thereof is administered orally once or more per day.

77. The method of embodiment 76, wherein Compound A or a pharmaceutically acceptable salt is administered orally once per day.

78. The method of embodiment 76 or 77, wherein Compound A is administered at a daily dose of from about 0.5 mg to about 50 mg.

79. The method of embodiment 76 or 77, wherein Compound A is administered at a daily dose of from about 1 mg to about 25 mg.

80. The method of embodiment 76 or 77, wherein Compound A is administered at a daily dose of from about 8 mg to about 25 mg.

81. The method of embodiment 76 or 77, wherein Compound A is administered at a daily dose of from about 1 mg to about 10 mg.

82. The method of embodiment 76 or 77, wherein Compound A is administered at a daily dose of from about 2 mg to about 6 mg.

83. The method of any of embodiments 76-82, wherein the Compound A has a volume based particle size with a Dv90 of between about 5 and about 200 microns as determined by a light scattering technique.

84. The method of any of embodiments 76-82, wherein the Compound A has a volume based particle size with a Dv90 of between about 5 and about 100 microns as determined by a light scattering technique.

85. The method of any of embodiments 76-82, wherein the Compound A has a volume based particle size with a Dv90 of between about 5 and about 50 microns as determined by a light scattering technique.

86. The method of any of embodiments 76-85, wherein the Compound A provides a Cₘₐₓ of Compound A of not more than about 50 ng/mL.

87. The method of any of embodiments 76-85, wherein the Compound A provides a Cₘₐₓ of Compound A of not more than about 40 ng/mL.

88. The method of any of embodiments 76-85, wherein the Compound A provides a Cₘₐₓ of Compound A of not more than about 35 ng/mL.

89. The method of any of embodiments 76-85, wherein the Compound A provides a Cₘₐₓ of Compound A of not more than about 30 ng/mL.

90. The method of any of embodiments 76-85, wherein the Compound A provides a Cₘₐₓ of Compound A of not more than about 25 ng/mL.

91. The method of any of embodiments 76-85, wherein the Compound A provides a Cₘₐₓ of Compound A of not more than about 20 ng/mL.

92. The method of any of embodiments 76-85, wherein the Compound A provides a Cₘₐₓ of Compound A of not more than about 15 ng/mL.

93. The method of any of embodiments 76-85, wherein the Compound A provides a Cₘₐₓ of Compound A of not more than about 10 ng/mL.

94. The method of any of embodiments 76-85, wherein the Compound A provides a Cₘₐₓ of Compound A of not more than about 5 ng/mL.

95. The method of any of embodiments 76-94, wherein the Compound A provides a Cₘₐₓ of Compound A of between about 5 ng/mL and about 35 ng/ml and a half-life (t_{1/2}) of Compound A between about 4 and about 12 hours.

96. The method of any of embodiments 76-94, wherein the Compound A provides a Cₘₐₓ of Compound A of between about 5 ng/ml and about 35 ng/ml and a half-life (t_{1/2}) of Compound A between about 6 and about 12 hours.

97. The method of any of embodiments 76-94, wherein the Compound A provides a Cₘₐₓ of Compound A of between about 5 ng/mL and about 35 ng/ml and a half-life (t_{1/2}) of Compound A between about 8 and about 12 hours.

98. The method of any of embodiments 76-94, wherein the Compound A provides a Cₘₐₓ of Compound A of between about 5 ng/mL and about 35 ng/mL and a half-life (t_{1/2}) of Compound A between about 8 and about 10 hours.

99. The method of any of embodiments 76-94, wherein the Compound A provides a first plasma concentration peak of Compound A of between about 5 ng/ml and about 50 ng/ml within about 4 hours and a second plasma concentration peak between about 8 and about 24 hours following oral administration.

100. The method of any of embodiments 76-99, wherein the plasma concentration for Compound A following oral administration is about 3.0 ng/mL or more at 2 hours after administration and about 1.0 ng/mL or less at 12 hours after administration.

101. The method of any of embodiments 76-99, wherein the plasma concentration for Compound A following oral administration is about 3.0 ng/mL or more at 2 hours after administration and about 1.5 ng/mL or less at 12 hours after administration.

102. The method of any of embodiments 76-99, wherein the plasma concentration for Compound A following oral administration is about 4.0 ng/ml or more at 2 hours after administration and about 1.5 ng/mL or less at 12 hours after administration.

103. The method of any of embodiments 76-99, wherein the plasma concentration for Compound A following oral administration is about 5.0 ng/ml or more at 2 hours after administration and about 1.5 ng/ml or less at 12 hours after administration.

104. The method of any of embodiments 76-99, wherein the plasma concentration for Compound A following oral administration is about 10.0 ng/ml or more at 2 hours after administration and about 2.0 ng/mL or less at 12 hours after administration.

105. The method of any of embodiments 76-99, wherein the plasma concentration for Compound A following oral administration is about 15.0 ng/ml or more at 2 hours after administration and about 2.0 ng/mL or less at 12 hours after administration.

106. The method of any of embodiments 76-99, wherein the plasma concentration for Compound A following oral administration is about 20.0 ng/ml or more at 2 hours after administration and about 2.0 ng/mL or less at 12 hours after administration.

107. The method of any of embodiments 76-99, wherein the plasma concentration for Compound A following oral administration is about 20.0 ng/mL or more at 2 hours after administration and about 4.0 ng/mL or less at 12 hours after administration.

108. A method for treating idiopathic hypersomnia in a subject in need thereof, comprising administering to the subject an effective amount of Compound A or a pharmaceutically acceptable salt thereof, wherein Compound A or a pharmaceutically acceptable salt thereof is administered orally once or more per day.

109. The method of embodiment 108, wherein Compound A or a pharmaceutically acceptable salt is administered orally once per day.

110. The method of embodiment 108 or 109, wherein Compound A is administered at a daily dose of from about 0.5 mg to about 50 mg.

111. The method of embodiment 108 or 109, wherein Compound A is administered at a daily dose of from about 1 mg to about 25 mg.

112. The method of embodiment 108 or 109, wherein Compound A is administered at a daily dose of from about 8 mg to about 25 mg.

113. The method of any of embodiments 108-112, wherein the Compound A has a volume based particle size with a Dv90 of between about 5 and about 200 microns as determined by a light scattering technique.

114. The method of any of embodiments 108-112, wherein the Compound A has a volume based particle size with a Dv90 of between about 5 and about 100 microns as determined by a light scattering technique.

115. The method of any of embodiments 108-112, wherein the Compound A has a volume based particle size with a Dv90 of between about 5 and about 50 microns as determined by a light scattering technique.

116. The method of any of embodiments 108-115, wherein the Compound A provides a Cₘₐₓ of

Compound A of not more than about 50 ng/mL.

117. The method of any of embodiments 108-115, wherein the Compound A provides a Cₘₐₓ of Compound A of not more than about 40 ng/mL.

118. The method of any of embodiments 108-115, wherein the Compound A provides a Cₘₐₓ of Compound A of not more than about 35 ng/ml.

119. The method of any of embodiments 108-118, wherein the Compound A provides a Cₘₐₓ of Compound A of between about 5 ng/ml and about 50 ng/ml and a half-life (t_{1/2}) of Compound A between about 4 and about 12 hours.

120. The method of any of embodiments 108-118, wherein the Compound A provides a Cₘₐₓ of Compound A of between about 10 ng/mL and about 50 ng/ml and a half-life (t_{1/2}) of Compound A between about 6 and about 12 hours.

121. The method of any of embodiments 108-118, wherein the Compound A provides a Cₘₐₓ of Compound A of between about 15 ng/ml and about 50 ng/ml and a half-life (t_{1/2}) of Compound A between about 8 and about 12 hours.

122. The method of any of embodiments 108-118, wherein the Compound A provides a Cₘₐₓ of Compound A of between about 15 ng/ml and about 50 ng/ml and a half-life (t_{1/2}) of Compound A between about 8 and about 10 hours.

123. The method of any of embodiments 108-122, wherein the Compound A provides a first plasma concentration peak of Compound A of between about 5 ng/mL and about 50 ng/ml within about 4 hours and a second plasma concentration peak between about 8 and about 24 hours following oral administration.

124. The method of any of embodiments 108-123, wherein the plasma concentration for Compound A following oral administration is about 15.0 ng/mL or more at 2 hours after administration and about 2.0 ng/ml or less at 12 hours after administration.

125. The method of any of embodiments 108-123, wherein the plasma concentration for Compound A following oral administration is about 20.0 ng/ml or more at 2 hours after administration and about 3.0 ng/mL or less at 12 hours after administration.

126. The method of any of embodiments 108-123, wherein the plasma concentration for Compound A following oral administration is about 20.0 ng/ml or more at 2 hours after administration and about 4.0 ng/mL or less at 12 hours after administration.

127. A pharmaceutical composition comprising a pharmaceutically active compound or a pharmaceutically acceptable salt thereof in an amount of from about 0.5 mg to about 50 mg, wherein the pharmaceutically active compound is Compound A.

128. The pharmaceutical composition according to embodiment 127, wherein the composition is adapted for administration once, twice or three times daily.

129. The pharmaceutical composition according to embodiment 127 or embodiment 128 wherein the pharmaceutically active compound is present in the form of particles having a Dv90 of between about 5 microns and about 200 microns as determined by a light scattering technique.

130. The pharmaceutical composition according to embodiment 129, wherein the pharmaceutically active compound particles have a Dv90 of between about 5 microns and about 25 microns as determined by a light scattering technique.

131. The pharmaceutical composition according to embodiment 128, wherein the composition is adapted for administration once daily.

132. The pharmaceutical composition according to embodiment 131, wherein the pharmaceutically active compound is present in the form of particles having a Dv90 of 12 microns ±25% as determined by a light scattering technique, or a specific surface area of 10 m²/g±25% as measured by the BET method.

133. The pharmaceutical composition according to embodiment 132, wherein the particles have a Dv90 of from 9.0 to 15.0 microns and/or a specific surface area of from 7.5 to 12.5 m²/g.

134. The pharmaceutical composition according to embodiment 133, wherein the particles have a Dv90 of about 12 microns and/or a specific surface area of about 8.8 m²/g.

135. The pharmaceutical composition according to any one of embodiments 127 to 134, wherein said composition is adapted for oral administration.

136. The pharmaceutical composition according to embodiment 135, wherein said composition is in the form of a powder, a granulate, a tablet, particles, pellets or a liquid containing any of the foregoing forms dispersed or dissolved therein.

137. The pharmaceutical composition according to embodiment 136, wherein the pharmaceutically active compound is presented in the form of a powder in a capsule.

138. The pharmaceutical composition according to embodiment 136, wherein the pharmaceutically active ingredient is presented in the form of a tablet.

139. The pharmaceutical composition according to embodiment 138, wherein said tablet further comprises one or more pharmaceutically acceptable excipients.

140. The pharmaceutical composition according to embodiment 139, wherein said tablet is in a form selected from the list consisting of: an immediate release tablet, a delayed release tablet, an extended release tablet and a controlled release tablet.

141. The pharmaceutical composition according to embodiment 140, wherein said tablet is an immediate release tablet.

142. The pharmaceutical composition according to embodiment 140, wherein said tablet is a delayed release tablet, an extended release tablet or a controlled release tablet, wherein said tablet further comprises one or more release rate modifying pharmaceutically acceptable excipients.

143. The pharmaceutical composition according to 142, wherein said tablet comprises a controlled release matrix or a controlled release coating.

144. The pharmaceutical composition according to embodiment 142, wherein said tablet comprises a controlled release matrix and a controlled release coating.

145. The pharmaceutical composition according to any one of embodiments 135 to 144 wherein the particle size distribution of the pharmaceutically active compound has a Dv10 of from 1 micron to 30 microns, a Dv50 of from 2 microns to 100 microns and a Dv90 of from 10 microns to 200 microns.

146. The pharmaceutical composition according to any one of embodiments 127 to 145 for use in the treatment of narcolepsy or a symptom thereof.

147. The pharmaceutical composition according to any one of embodiments 127 to 145, for use in the treatment of a condition selected from the list consisting of narcolepsy type 1, narcolepsy type 2, idiopathic hypersomnia, excessive daytime sleepiness, cataplexy or combinations thereof.

148. The pharmaceutical composition according to embodiment 147, for use in the treatment of narcolepsy type 1.

149. The pharmaceutical composition according to embodiment 147, for use in the treatment of narcolepsy type 2.

150. The pharmaceutical composition according to embodiment 147, for use in the treatment of idiopathic hypersomnia.

151. The pharmaceutical composition according to embodiment 147, for use in the treatment of excessive daytime sleepiness.

152. The pharmaceutical composition according to embodiment 147, for use in the treatment of cataplexy.

153. The pharmaceutical composition according to any one of embodiments 135 to 145, which upon oral administration to a subject provides a Cₘₐₓ of not more than 50 ng/ml.

154. The pharmaceutical composition according to any one of embodiments 135 to 145, which upon oral administration to a subject provides a dual absorption plasma concentration profile such that there is a first plasma concentration peak of between about 5 ng/ml and about 50 ng/ml within about 4 hours and a second plasma concentration peak between about 8 and about 24 hours.

155. The pharmaceutical composition according to any one of embodiments 135 to 145 or 154 wherein the plasma concentration of the pharmaceutically active compound following oral administration is about 3.0 ng/mL or more at 2 hours after administration and about 2.0 ng/ml or less at 12 hours after administration.

While this invention has been particularly shown and described with references to particular embodiments thereof, it will be understood by those skilled in the art that various changes in form and details may be made therein without departing from the scope of the invention encompassed by the appended claims.

1. A method for treating narcolepsy in a subject in need thereof, comprising administering to the subject an effective amount of Compound A 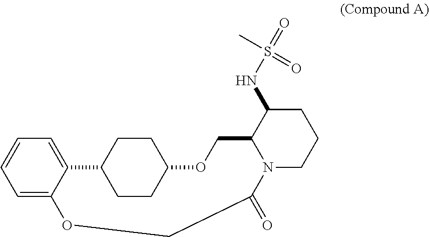
or a pharmaceutically acceptable salt thereof, wherein Compound A or a pharmaceutically acceptable salt thereof is administered orally once or more per day.

2. The method of claim 1, wherein Compound A or a pharmaceutically acceptable salt is administered orally once per day.

3. The method of claim 1, wherein Compound A is administered at a daily dose of from about 1 mg to about 25 mg.

4. The method of claim 1, wherein the Compound A has a volume based particle size with a Dv90 of between about 5 and about 200 microns as determined by a light scattering technique.

5. The method of claim 1, wherein the Compound A provides a Cₘₐₓ of Compound A of between about 5 ng/ml and about 35 ng/mL and a half-life (t_{1/2}) of Compound A between about 4 and about 12 hours.

6. The method of claim 1, wherein the plasma concentration for Compound A following oral administration is about 3.0 ng/mL or more at 2 hours after administration and about 1.0 ng/mL or less at 12 hours after administration.

7. A method for treating narcolepsy type 1 in a subject in need thereof, comprising administering to the subject an effective amount of Compound A 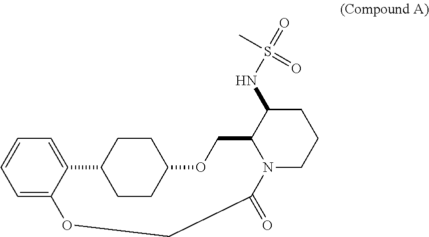
or a pharmaceutically acceptable salt thereof, wherein Compound A or a pharmaceutically acceptable salt thereof is administered orally once or more per day.

8. The method of claim 7, wherein Compound A or a pharmaceutically acceptable salt is administered orally once per day.

9. The method of claim 7, wherein Compound A is administered at a daily dose of from about 1 mg to about 10 mg.

10. The method of claim 7, wherein the Compound A has a volume based particle size with a Dv90 of between about 5 and about 200 microns as determined by a light scattering technique.

11. The method of claim 7, wherein the Compound A provides a Cₘₐₓ of Compound A of between about 5 ng/mL and about 35 ng/ml and a half-life (t_{1/2}) of Compound A between about 4 and about 12 hours.

12. The method of claim 7, wherein the plasma concentration for Compound A following oral administration is about 3.0 ng/mL or more at 2 hours after administration and about 1.0 ng/mL or less at 12 hours after administration.

13. A method for treating narcolepsy type 2 in a subject in need thereof, comprising administering to the subject an effective amount of Compound A 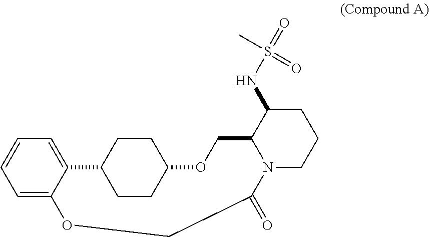
or a pharmaceutically acceptable salt thereof, wherein Compound A or a pharmaceutically acceptable salt thereof is administered orally once or more per day.

14. The method of claim 13, wherein Compound A or a pharmaceutically acceptable salt is administered orally once per day.

15. The method of claim 13, wherein Compound A is administered at a daily dose of from about 8 mg to about 25 mg.

16. The method of claim 13, wherein the Compound A has a volume based particle size with a Dv90 of between about 5 and about 200 microns as determined by a light scattering technique.

17. The method of claim 13, wherein the Compound A provides a Cₘₐₓ of Compound A of between about 5 ng/ml and about 35 ng/ml and a half-life (t_{1/2}) of Compound A between about 4 and about 12 hours.

18. The method of claim 13, wherein the plasma concentration for Compound A following oral administration is about 3.0 ng/mL or more at 2 hours after administration and about 1.0 ng/mL or less at 12 hours after administration.

19. A method for treating idiopathic hypersomnia in a subject in need thereof, comprising administering to the subject an effective amount of Compound A 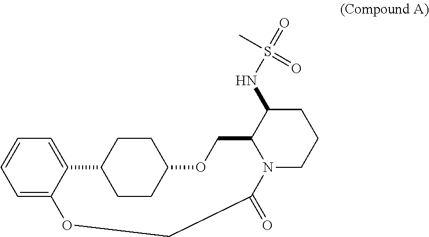
or a pharmaceutically acceptable salt thereof, wherein Compound A or a pharmaceutically acceptable salt thereof is administered orally once or more per day.

20. The method of claim 19, wherein Compound A or a pharmaceutically acceptable salt is administered orally once per day.

21. The method of claim 19, wherein Compound A is administered at a daily dose of from about 8 mg to about 25 mg.

22. The method of claim 19, wherein the Compound A has a volume based particle size with a Dv90 of between about 5 and about 200 microns as determined by a light scattering technique.

23. The method of claim 19, wherein the Compound A provides a Cₘₐₓ of Compound A of between about 5 ng/ml and about 35 ng/ml and a half-life (t_{1/2}) of Compound A between about 4 and about 12 hours.

24. The method of claim 19, wherein the plasma concentration for Compound A following oral administration is about 3.0 ng/mL or more at 2 hours after administration and about 1.0 ng/mL or less at 12 hours after administration.

25. A pharmaceutical composition comprising a pharmaceutically active compound or a pharmaceutically acceptable salt thereof in an amount of from about 0.5 mg to about 50 mg, wherein the pharmaceutically active compound is Compound A: 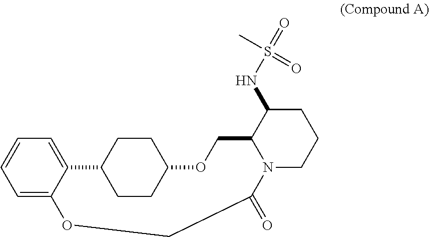
or a pharmaceutically acceptable salt thereof.

26. The pharmaceutical composition according to claim 25, wherein the composition is adapted for administration once, twice or three times daily.

27. The pharmaceutical composition according to claim 25, wherein the pharmaceutically active compound is present in the form of particles having a Dv90 of between about 5 microns and about 200 microns as determined by a light scattering technique.

28. The pharmaceutical composition according to claim 27, wherein the pharmaceutically active compound is present in the form of particles having a Dv90 of 12 microns±25% as determined by a light scattering technique, or a specific surface area of 10 m²/g±25% as measured by the BET method.
